SEQUENCE LISTING (20 sequences)

SEQ ID NO: 1 - protein, 720 aa, unidentified
  source 1..720: note = Gossypium raimondii
METITQNGHHKGSLESFCTASKGAGGAVDPLNWGVAAESLKGSHLDEVKRMVAEYRNPLVKLGGETLTISQVAAIATRDLGVKVELSEDARAGVKASADWVLDGMNKGTDSYGVTTGFGATSHRRTKQGAALQKELIRFLNAGVFGHGTESCHTLPHSATRAAMLVRINTLLQGYSGIRFEILEAITKLINSNITPCLPLRGTITASGDLVPLSYIAGLLTGRPNSKAVGPNGEPLDAEEAFRVAGIDSGFFVLQPKEGLALVNGTAVGSGMASMVLFEANILAVLSEVLSAIFAEVMNGKPEFTDHLTHKLKHHPGQIEAAAIMEHILEGSSYVKAAKKLHEMDPLQKPKQDRYALRTSPQWLGPQIEVIRFATKSIEREINSVNDNPLIDVSRNKALHGGNFQGTPIGVSMDNARLAIAAIGKLMFAQFSELVNDFYNNGLPSNLSGGRNPSLDYGFKGAEIAMASYCSELQYLANPVTSHVQSAEQHNQDVNSLGLISSRKTAEAVDILKLMSSTYLAALCQAIDLRHLEENLRSTVKNTVSQIAKKVLTTGANGELHPSRFCEKDLLKAVDREYVFAYIDDPCSATYPLMQKLRQVLVEHALTNGENEKNASTSIFQKIAAFEEELKAVLPKEVESARASVENGNAAIPNKIKECRSFPLYKFVREELGTGLLTGENVMSPGEEFDKVFTAMCQGRIIDPMLECLEEWNGAPLPIC

SEQ ID NO: 2 - DNA, 2160 bp, synthetic construct
  source 1..2160: mol_type = other DNA
atggaaaccattacgcaaaatggccaccataaaggttccctagagtcgttttgtacagcaagcaagggggccggcggtgcggtcgatcctttgaactggggtgtagctgctgaatctttaaaaggctcacacctggacgaagttaaacgtatggttgcagagtatagaaacccgctggtgaagcttgggggagaaactctcaccatctctcaggtggcggctatcgctacccgcgatctgggtgtaaaagttgaactgagcgaggacgctcgtgcgggcgtaaaagcatccgctgattgggttctggacggtatgaacaaaggcactgactcttacggtgtcaccactggtttcggcgcgactagccatcgacgcacaaaacagggcgccgccctgcaaaaggaactgatacgcttcctgaacgcgggagtgtttggtcacggcaccgaaagttgccacaccctgccccattcggcaactcgtgctgctatgctggtccgtatcaatacgctcctgcaaggttactctggcattcgtttcgaaatcctggaagctattactaaactcatcaacagcaacattaccccgtgcttgccgctgcgcggtacgatcactgcgtctggcgatctggttccattatcctatatcgcaggcctgctgacgggtcgtccgaacagcaaggccgttggccctaacggtgagccgctggacgctgaagaggcattccgtgtagccgggattgattctggatttttcgttctgcaaccaaaagaaggcctggcgctggtgaatggtaccgctgttggttccggcatggctagcatggtgctgtttgaagctaacatcctggcggtgctgtctgaggtactgagcgcaatcttcgcagaagttatgaacggtaaaccggagttcacagaccaccttactcataaacttaaacaccatccggggcagatcgaagcggcagcgattatggaacacatactggaaggtagttcctacgtaaaagctgcaaaaaagctacacgaaatggatccattgcagaaaccgaaacaggaccgctacgccctccgcacctcgccgcagtggctgggcccgcagattgaggtaatccgtttcgctaccaaatctatcgaaagggaaatcaactctgttaacgacaacccgttaatcgatgtctctcgtaacaaggcactgcacggcggtaattttcagggaactccaattggtgtttctatggataacgctcggcttgctattgctgccatcggtaaactgatgttcgcccagttctccgagctggtaaacgatttctataacaatggcttgccgagcaatctgagcggcgggaggaacccgtctttggactacggtttcaaaggtgcagaaatcgcgatggctagttattgcagtgaactgcaatacctggctaatccggtaacctcccacgtgcagtccgcagagcagcacaaccaggacgtgaacagcctgggcctgatcagctcccgcaaaactgcggaagctgttgatattctgaagctgatgtcgtcaacttacttagcggcgctgtgtcaggcaatcgacctgcgtcatctggaagaaaacctgcgttctaccgttaaaaacactgtttcgcagattgctaaaaaggttctgacgaccggtgcgaatggcgaactccacccttctcgtttttgcgaaaaagacctgctaaaagccgtggatcgtgaatatgtctttgcgtacatcgacgatccgtgtagcgctacgtacccactgatgcagaaactgcgccaagtgctggttgaacatgcactgaccaacggggaaaacgagaaaaacgctagcaccagtatcttccagaagatagcggcattcgaggaagagctgaaagcggttcttcccaaagaggttgaatcggcgcgtgcgtccgtagaaaatggaaacgctgccattccgaacaaaatcaaagaatgccgctcttttccgttatacaaatttgtgcgtgaagagctgggcactggtttgctgacgggcgagaacgttatgtcccccggtgaagaatttgacaaggtcttcaccgctatgtgccaggggcgtatcatcgatccaatgttagaatgcctggaggaatggaacggcgcccctctaccgatttgc

SEQ ID NO: 3 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 4 - DNA, 1701 bp, synthetic construct
  source 1..1701: mol_type = other DNA
atgaaaacactgtcccaagcacagtcgaagacgagtagccagcagttttctttcaccggtaattctagcgctaacgtgattatcggcaaccagaaactaactatcaacgacgtcgcccgcgttgctcggaacgggaccttagtaagcctgactaacaacactgatatacttcaaggcattcaggcgtcctgtgattatatcaataacgcagttgagtctggtgaaccaatttacggtgtgacctcaggcttcggcggtatggcgaacgttgctatcagccgtgaacaggcctccgaactgcaaaccaatttggtatggtttctgaaaactggtgctgggaacaaactccctttggcggacgtacgtgcagcaatgctgctgcgcgccaactcgcatatgcgtggcgcgtccggtatccgtctggagctgatcaaacgtatggaaatcttcttgaacgctggtgttactccgtacgtttatgaatttggatctatcggcgcttctggagatctggtcccgctgagctacattacgggttccctgattggccttgacccgagcttcaaggtggatttcaacggcaaagaaatggacgccccgaccgcgttacgccagttaaatctgtctcccctgacacttctgcctaaagaaggtctagctatgatgaatgggacctcagtcatgactggcatcgcagctaactgcgtatacgacacccagatcctgactgcgattgcaatgggtgttcacgctctggatatccaggccctgaacggcaccaaccagtctttccacccgtttatccataactctaagccgcacccaggtcagctgtgggcggctgatcagatgatatcattgctggctaactcgcaactggtacgggacgagctggacggcaaacatgattaccgcgaccacgagctgatccaggatcgttatagcctgcgttgccttccgcagtacctgggtccgattgtggacggtatctcacagatagcaaaacaaatcgaaattgaaattaactccgttactgataaccctctgattgacgtcgataaccaggcgtcgtaccacggcggaaatttcctgggtcagtatgttggcatgggtatggaccaccttcgctactatatcggcctgctggcgaaacacctggatgtgcagattgcgctgctagctagtcccgaatttagcaacggactgccgccatctttattgggcaaccgtgaacgtaaggttaacatgggtctgaaaggtttacaaatctgtggcaattccatcatgccgctgctgacgttctacggcaatagcatcgccgaccgctttccgacccatgcagagcaattcaaccagaatatcaactctcagggctacacctccgcaacgctggcgcgacgtagtgttgatatcttccaaaactacgttgcgattgccctgatgtttggcgtccaggctgtagacctgaggacttataaaaagactggccattacgatgcgcgtgcttgcctctctccggctaccgaacgcctgtattccgccgtgcgtcacgtagttggtcagaaacctacttcagatcgcccatacatctggaacgataacgagcagggtctggatgaacacatcgctcgcatctccgctgacattgccgctggcggagtaattgttcaagctgtacaggatatcctgccgtgcctgcac

SEQ ID NO: 5 - protein, 496 aa, Rhizobium radiobacter
MTVLLDDGLSWRDVAHIGKGEALRVSDAAYLRIDRASQIVDSIVESGVRAYGINTGVGALSDTVVDRTAQGRLSRSIILSHACGVGRLLDPREVRAIMAAQIANFAHGHSGVRREIVSHLSTFLEQDCIPDVPSRGSAGYLVHNAHVALVLIGEGQARLGGRVMSGRDALAAMGLQPIVLGAKEGLSLVNGTACATGLSCMALARANHLLDWADAIAALTLEAAGCQIDAFDKTVLALRPSKGIAAVGAALRSRLEGSGLVAAAHGRRTQDALSLRAVPHAHGAARDIFDACASIVDRELASVTDNPAILGTPEQPIVSSEAHAVAPALGQAADSLAIAIAQIASMSERRIDRLVNPLVSGLPPFLATDAGSHSGFMIAQYTAAALVGDSRRLSAPASTDGGLTSGLQEDFLSHPTAAANKLLAVLDNAEYILAIEWMAGVQAHDSLESVAGRAAGTNVVYNLLREHLQPYSDDRPLSADMEKARLLLRDLSPPDM

SEQ ID NO: 6 - DNA, 1488 bp, synthetic construct
  source 1..1488: mol_type = other DNA
atgaccgttctcttggatgacggtctgtcttggcgggacgtggcgcatattggcaaaggtgaagcacttcgcgtaagcgatgctgcctatctgcgtatcgatcgtgctagtcaaatcgtcgactccattgttgagtcaggcgtacgcgcatacgggataaatacgggtgtgggcgctctgtcggacactgttgttgatcgtacagcgcagggaagattatctcgttccatcatcctgagccacgcgtgcggtgtcggccgcctgctggacccgagggaagtgcgtgctattatggcagctcagatcgccaactttgcacacggtcactctggcgttcgtcgtgaaatcgtaagtcatctgagcactttcctggaacaggattgtatcccagacgttccctcccgcggctctgctggctacctggttcataacgcacacgtcgcgttggtactgattggtgagggtcaggcccgtctgggtggtcgcgttatgagcgggcgtgatgccctggctgcgatggggctccagccgatagtgctgggcgcgaaggaaggactgtcgttagtaaacggtaccgcctgcgctaccggcttatcttgcatggcgctggctcgagcaaaccacctgttggactgggctgatgctatcgctgcactgactctggaagcagcgggctgtcaaatcgacgcattcgataaaacggttctggcgctgcgcccgagcaaaggtattgctgctgttggcgcagccctccgttcacgtctggagggttccggactggtggcggctgcacacggccgccgtactcaggacgcgctgtccctgcgcgctgttcctcatgcacacggtgcagcgcgcgatatctttgacgcatgcgcttctatcgtcgatcgtgaactggcgagcgtgaccgacaatccggcaattcttggcactccggagcagcctatcgtatcttccgaagctcacgctgttgccccggctctgggtcaggctgccgacagcctagcgattgcgattgcacaaatcgccagcatgtctgaaaggcgtatcgatcggctagttaacccgctggtcagcggacttccgccattcttggcgaccgatgctggtagccactcaggcttcatgattgcgcagtatactgctgcggctctggtaggcgattcgcgccgtctgtcggcccctgcgtctactgacggtggcctgacctccggtcttcaagaagactttctgtctcatccgaccgccgctgcaaacaaacttctagcggtactggacaatgctgaatacatcctggccatcgagtggatggctggtgttcaggcgcacgatagtctggagtccgtagcggggcgcgcggctggcacgaacgtggtatacaaccttttgcgtgaacacctacagccatatagtgacgatcgaccgctgtctgccgatatggaaaaggcgcgtctgctgctgcgtgacctgtcccctccggacatg

SEQ ID NO: 7 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 8 - DNA, 2151 bp, synthetic construct
  source 1..2151: mol_type = other DNA
atggaccaaatcgaggctatgctgtgtgggggaggcgaaaaaacaaaggttgcagtaacgactaaaaccttagccgatcctcttaactggggtctggctgcggaccagatgaaaggttctcatttggatgaagtgaagaaaatggtcgaagaatatcgccgtcccgttgttaatctaggcggcgagactctgaccattggtcaggtggcagcgatcagcaccgtaggcggttcagttaaagttgaactcgctgaaacttcgcgtgctggtgtgaaagcatccagcgattgggtgatggagtctatgaacaaaggcactgactcctacggagtaaccacgggttttggcgcgacttctcaccgccgtaccaagaacggtaccgctctgcaaacagaactgatacgattcctgaatgccggtatcttcggcaacactaaagagacttgccacaccctgccacagagcgcgactcgtgctgctatgctggttagggtgaacaccctgctgcaagggtacagtggcattcgtttcgaaatcctggaagcaattacatcgctgctgaaccataatatctcaccgagcttgccgctgcgcggtacgatcaccgcatccggtgacttagtcccgctgtcttatatcgcagggctgttgacgggtcgcccgaactccaaagccactggcccggatggcgagagcctgaccgctaaagaagcgtttgaaaaggccggcatctctactggtttcttcgacctgcaaccgaaagaaggtcttgcactggtaaacggcaccgccgtaggcagtggtatggcatctatggtgctgtttgaagctaacgttcaggctgttctggcggaagttttatccgcgattttcgctgaggtgatgagcggtaaaccggaatttacggatcacctcacccaccgcttaaaacatcaccctggccagattgaagccgctgcaatcatggaacacattctcgacggttcttcctacatgaaattggctcagaaagtccatgagatggacccactgcaaaaaccaaaacaggatcgttacgcgctgcgtacttctccgcagtggctaggaccgcagatcgaagttatccgtcaggctaccaagtccattgaacgagaaattaacagcgtcaacgacaaccccctgattgatgtatcgcgcaacaaggcaatccacggcggtaacttccaggggacaccgatcggcgttagcatggataatactcgtttagccatcgcagcaatcggtaagctgatgtttgctcagttctctgagctggttaatgacttttataacaacgggcttccttcgaatctcaccgctagctccaacccgtctttagactacggtttcaaaggcgctgaaatcgcgatggcgtcctattgctcagaactgcaatacctggcgaacccggtgacctcacatgtgcagagcgctgagcagcacaaccaggatgtcaactctctgggcctgataagttcaaggaaaacttcagaagctgtcgacatccttaagctcatgtctacgaccttcctggtaggcatctgccaggctgttgatctgcgtcacctggaggaaaacttacgtcagactgttaaaaacactgtaagccaagtcgctaaaaaagttctgacgaccggcatcaacggcgagctgcacccttcccgcttctgcgagaaggatctactgaaagttgtggaccgtgaacaggttttcacttacgttgacgatccgtgttctgcgacttacccattgatgcagcggctgcgccaggtgatagttgatcatgcactgagcaatggtgaaaccgaaaagaacgctgttactagcatttttcaaaaaatcggtgctttcgaggaagagctgaaagcggtactgccgaaagaagtggaagccgcgcgtgctgcgtatggcaacggtaccgcaccgatcccgaaccgtatcaaggaatgccgtagttatccactgtaccgcttcgtacgtgaagaactgggtaccaagctgttaaccggggaaaaagttgtttctcctggcgaagaatttgacaaagtatttacggccatgtgcgaaggcaaactgattgacccgctgatggattgcctaaaagaatggaacggtgctcccatcccgatttgt

SEQ ID NO: 9 - protein, 720 aa, Capsicum annuum
MASIAQNGHVNGDVVAIDFCKKSIHDPLNWEMAAESLKGSHLDEVKKMVDEFRKPIVKLGGETLTVAQVASIANADNKTCGAKVELSERARAGVKASSDWVMDSMCKGTDSYGVTTGFGATSHRRTKNGGALQKELIRFLNAGVFGNGTESCHTLPHSATRAAMLVRINTLLQGYSGIRFEILEAITKLINSNITPCLPLRGTITASGDLVPLSYIAGLLTGRPNSKAVGPNGEKLNAEEAFRVAGVSGGFFELQPKEGLALVNGTAVGSGMASMVLFESNILAVMSEVLSAIFAEVMNGKPEFTDHLTHKLKHHPGQIEAAAIMEHILDGSSYVKAAQKLHEMDPLQKPKQDRYALRTSPQWLGPQIEVIRAATKMIEREINSVNDNPLIDVSRNKALHGGNFQGTPIGVSMDNTRLALASIGKLMFAQFSELVNDYYNNGLPSNLTAGRNPSLDYGFKGAEIAMASYCSELQFLANPVTNHVQSAEQHNQDVNSLGLISARKTAEAVDILKLMSSTYLVALCQAIDLRHLEENLKNAVKNTVSQVAKRTLTMGANGELHPARFCEKELLRVVDREYLFAYADDPCSSTYPLMQKLRQVLVDHALNNGESEKNVNSSIFQKIAAFEDELKAVLPKEVESARITLESGNPSIPNRITECRSYPLYRLVRKELGTELLTGERVRSPGEEIDKVFTAMCNGQIIDPLLECLKSWNGAPLPIC

SEQ ID NO: 10 - DNA, 2160 bp, synthetic construct
  source 1..2160: mol_type = other DNA
atggcaagcattgctcagaacggtcatgtgaatggggacgttgtagccatcgatttttgtaaaaagtccatccacgatccactgaactgggaaatggctgcggagtcattgaaaggctcgcaccttgacgaagttaaaaagatggtcgacgaatttcgtaaacctatcgttaaactcggtggcgagactctgacggtagcacaagtggcttctattgcgaacgcagataataaaacctgcggtgctaaagtcgaattatctgaacgcgctcgtgcaggcgtgaaggcgagcagtgactgggttatggattctatgtgcaaagggacagactcctacggtgtaactaccggcttcggtgccacttctcaccgccgtaccaaaaacggtggcgcgctgcaaaaagaactgatacggttcctgaacgctggcgttttcggcaacggtaccgagagctgccatactctgccgcactccgctacccgtgcggcgatgctagtgagaatcaatactctcctgcaaggatattcgggtatccgtttcgaaattctggaagctattacaaagctgatcaacagcaacatcactccgtgtctgccgctgaggggcaccatcacggcctccggagacttggttcccctttcttacatcgctggtctgctgacagggcgcccgaacagcaaagccgtgggccctaacggcgaaaagctaaatgcagaggaagcgttccgagttgctggtgtatctggtggttttttcgaacttcagccgaaagagggcttagcactggttaacggcaccgctgtcggttccggtatggcatcaatggtactgttcgaatcaaacattctggctgtaatgagtgaggtgttgtctgccatctttgctgaagttatgaacggcaaaccggaatttactgatcacctcacgcataagctgaaacaccacccaggccagatcgaggcggctgcgattatggaacatatactggatggctccagctatgtcaaagcagcacagaaactgcacgaaatggaccctctgcaaaaaccgaaacaggaccgctacgcgctgcgtaccagcccgcagtggctggggccgcagatcgaagtcatccgtgccgctactaaaatgattgaacgcgaaatcaacagcgttaatgataacccgctgatcgacgtatcgcgtaacaaagcactgcatggtggtaatttccagggcactcctattggggtgtccatggacaacacccgtctggccctggcatcaattggtaaactgatgtttgcccagttcagcgaacttgttaacgattactataataacggtctgccgtctaacctgacggcgggtcgcaacccatctctcgactacggcttcaagggtgctgagatcgcaatggctagttactgctctgagctgcaattccttgcgaacccagtcaccaatcacgttcagtcagccgagcagcacaaccaggatgttaactccctgggcttgatctctgcacgcaaaacggccgaagctgtagatatcctgaaactaatgagttctacttatctggttgctttatgccaggctattgacctgcgtcatcttgaagaaaatctgaagaacgcggtaaagaacacagtgagccaggttgcaaaacgtactctaaccatgggtgccaacggcgagctgcacccggctcgtttctgtgaaaaagaactgctgcgtgttgtggatcgagagtacttattcgcctacgcggatgacccgtgcagcagcacctatccgctgatgcagaagctgcgtcaggtactggtggatcatgcactgaacaacggcgaaagcgaaaagaatgttaacagctctatctttcagaaaatcgctgctttcgaagatgagctcaaagcggtgctgcccaaagaagtggaatctgctcgcattacactggaatctgggaatccatctataccgaaccgtatcactgagtgtcgcagctacccgctttatcgtttggtgcgcaaagagctgggcactgaattgctgaccggcgaacgtgttcgctcccccggtgaagaaattgataaagtattcaccgccatgtgcaacggtcaaatcatcgacccgctgttagaatgcctgaaatcctggaacggcgctccgctgccgatctgc

SEQ ID NO: 11 - protein, 711 aa, unidentified
  source 1..711: note = Salvia miltiorrhiza
MAAENGHHEESNGFCVKQNDPLNWVAAAESLKGSHLDEVKRMVEEFRKPVVKLGGETLTISQVAAIAAKDNAVAVELVESSRAGVKASSDWVMESMSKGTDSYGVTTGFGATSHRRTKQGGALQKELIRFLNAGIFGNGTESNHTLPHTATRAAMLVRINTLLQGYSGIRFEILEAITKFLNENITPCLPLRGTITASGDLVPLSYIAGLLTGRPNSKAVGPNGEPLNAEEAFKLAGVKGGFFELQPKEGLALVNGTAVGSGLASIALFDANILAVLSEVMSAVFAEVMNGKPEFTDHLTHKLKHHPGQIEAAAIMEHILDGSGYVKAAQKLHEQDPLQKPKQDRYALRTSPQWLGPQIEVIRTATKMIEREINSVNDNPLIDVSRNKALHGGNFQGTPIGVSMDNARLAIASIGKLLFAQFSELVNDLYNNGLPSNLSGGRNPSLDYGFKGSEIAMASYCSELQFLANPVTNHVQSAEQHNQDVNSLGLISSRKTVEALDILKLMSSTYLVALCQAVDLRHLEENLKHAVKNTVSQVAKRTLTMGVNGELHPSRFCEKDLIRVVDREYVFAYIDDPSSATYPLMQKLRQVLVDHALKNGDLEKNASTSIFQKIEAFEEELKALLPKEVGSARMALESGSPTVANRIAECRSYPLYKFIREQLGAGFLTGEKAVSPGEECEKVFTALSNGLIIDPLLECLQGWNGQPLPIC

SEQ ID NO: 12 - DNA, 2133 bp, synthetic construct
  source 1..2133: mol_type = other DNA
atggcggccgagaatggccaccatgaagaaagcaacggtttctgcgttaaacaaaacgacccactcaactgggtcgctgctgcggaatcgttgaaggggtcacacttagacgaagtaaaaaggatggtggaggaatttcgcaaacctgtggttaaacttggcggtgaaacgctgacaatcagtcaggtagcagctattgcagctaaggataacgcagttgctgttgagctggtggaatcctctcgggcgggtgttaaagccagctccgattgggtgatggagtctatgagcaaaggcactgactcttatggagttactaccggcttcggtgccacctcacaccgtcgtactaaacagggtggcgcgctgcaaaaggaactgatccgctttctgaatgctgggatattcggtaacggcaccgaatccaaccataccctgccgcacacggctactcgagcagctatgctcgtacgtatcaataccctgctgcaaggttacagcgggattcgtttcgaaatcctggaggccatcactaaatttttgaacgaaaacattaccccctgtctgccactgcgcggcaccatcactgcatctggcgatctggtcccgctatcttacattgcaggcctgttgactggccgtccgaactccaaagcggtaggtccgaacggtgaaccgctgaacgccgaagaggctttcaagctggctggtgttaaaggtggatttttcgaattacagcctaaagaaggcctggctctggtcaatggtacggcggttgggtctggactggcctcgattgctctgttcgatgcgaacatcctggctgtgctgagcgaagtgatgagtgccgttttcgcggaagtgatgaacggcaaaccggaatttacagaccatcttactcacaaactgaaacatcacccgggtcagatcgaggcggcagcaatcatggaacatattctcgatggtagcggctatgtaaaagctgcgcagaagctacacgaacaggacccgctgcaaaaaccaaaacaggaccgctacgccctgcgtacttccccgcagtggctgggtccccagatcgaggttatccgtaccgcaaccaagatgatcgagcgagagattaattctgtaaacgataacccgctgatcgatgtttcccgtaataaagcactgcacggcggcaactttcagggtactccgattggcgtgtctatggacaacgctcgccttgcgatcgctagcatcggtaagctgttgttcgctcagttcagcgaactggtaaacgacttgtacaacaatggactgccgtctaacttatcaggtggacgcaacccgtccctggattatgggtttaagggctccgaaatcgcgatggctagctactgctccgaactgcaattcttagccaaccctgtcaccaatcacgtccagagcgctgagcagcacaaccaggacgttaactcgctgggcctgattagttcccgtaaaacggtggaagctctcgatatcctgaaactgatgtcgtctacgtatcttgttgccctgtgccaggcagtagatctgcgccatctggaagaaaacctgaaacacgcagtgaaaaacaccgtcagtcaagttgccaaacgtaccctgacaatgggggttaacggcgaactgcatccttctcgtttctgtgaaaaagacttgataagagtagttgaccgcgaatacgtctttgcttacatcgatgacccatcatctgcaacttatccgctgatgcagaagctgcgtcaggttctggttgatcatgcactaaaaaacggtgacctcgagaagaacgcgagcacctcaattttccaaaaaatcgaggccttcgaagaagaactgaaagctttactgcctaaagaagtaggctctgcaagaatggcgcttgagagcggttccccgactgtggctaaccgtattgcggagtgccgtagttacccgctatataagttcattcgtgaacagctgggtgcgggcttcctgacaggtgaaaaagctgtttctccgggggaagaatgcgaaaaggtttttacggctctctccaatggcctcatcatcgatccgcttctggaatgtctgcaaggttggaacggccagccattgcctatatgc

SEQ ID NO: 13 - protein, 714 aa, Ricinus communis
MAAMAENGSKNDSLESFCNMGRDPLSWGLAAESMKGSHLDEVKKMVAEYRKPFVKLGGETLTVAQVAAIASHDCGVKVELSESARAGVKASSDWVMDSMNKGTDSYGVTTGFGATSHRRTKQGGALQKELIRFLNAGIFGNGTESCHTLPHSATRAAMLVRINTLLQGYSGIRFEILEAITKLLNHNITPCLPLRGTITASGDLVPLSYIAGLLTGRPNSKAIGPNGESMDALEAFRLAGIESGFFELQPKEGLALVNGTAVGSGLASMVLFEANILAVLSEILSAIFAEVMNGKPEFTDHLTHKLKHHPGQIEAAAIMEHILDGSSYVKAAKKLHEMDPLQKPKQDRYALRTSPQWLGPQIEVIRFSTKSIEREINSVNDNPLIDVSRNKALHGGNFQGTPIGVSMDNARLAIASIGKLMFAQFSELVNDFYNNGLPSNLTAGRNPSLDYGFKGAEIAMASYCSELQYLANPVTSHVQSAEQHNQDVNSLGLISSRKTAEAVDILKLMSTTYLVALCQAIDLRHLEENLRQAVKNTVSQVAKRVLTTGANGELHPSRFCEKDLLKVVDREYVFAYADDPCSATYPLMQKLRQILVEHALANGENEKNAGTSVFQKISAFEEELKTLLPKEVESVRIAYESGNPATANRIKECRSYPLYKFVREELGTGLLTGDKVMSPGEEFDKVFTAMCQGKIIDPMMDCLKEWNGAPLPIC

SEQ ID NO: 14 - DNA, 2142 bp, synthetic construct
  source 1..2142: mol_type = other DNA
atggcagctatggccgagaacggtagtaaaaatgattcactggaatcgttctgtaacatgggccgcgacccattatcttggggtttggcggctgaatctatgaagggcagccatctggacgaagtaaaaaagatggtggcggagtatcggaaaccttttgttaaactgggcggggaaacactgacggttgctcaggtcgcagctatcgcgtcccacgattgcggtgttaaagtagaactgagcgaatctgcccgtgcaggagttaaagctagctccgactgggtgatggatagcatgaacaaaggcaccgacagctacggtgtcactactggtttcggcgcaacctcccacaggcgcaccaagcaaggaggtgctctccagaaagagctaattcgttttctgaatgctggcatcttcggtaacggcactgaatcttgccacaccctgccccatagtgcgactcgtgcagcgatgctggtgcgcattaacacgctgcttcagggttacagcggcatccgtttcgagatcctcgaagctattaccaaactgctgaaccacaatatcactccgtgccttccgttacgtggtacaatcaccgcctctggagatttggttccgctgagctacattgccggcctgttaaccggtcgtccgaactccaaagcgatcggtccgaacggggaatctatggatgcgctggaagcttttcgactagccggcatagaatcaggtttcttcgaactgcaaccgaaggaaggcctggcactggtgaacggcactgctgttgggagtggtctggcctccatggtactgttcgaggcgaatatcctggcagttttatctgaaatcctgtcggccatttttgcagaagtaatgaacggcaaaccggaatttacggaccatctgactcacaagctgaaacaccatccaggtcagattgaggctgctgcaatcatggaacacatacttgatggctcctcttatgtcaaagcggctaaaaaattacatgagatggaccctctgcaaaagccgaaacaggaccgttacgctttgcgcaccagccctcagtggctggggccacagatcgaagtaatccgtttctcaactaaatccatcgaacgcgagattaactctgtcaacgataacccgctgatcgatgttagccgaaacaaggcactgcacggtggcaactttcagggcacccctattggcgttagtatggataatgctcgtctggcaatcgcgtctatcggtaaattaatgttcgcgcagttctccgagctcgtaaatgacttttacaacaacggtctgccgtcaaacctgaccgctggccgtaatccgtcgctcgactatggtttcaagggcgctgaaatcgctatggcgagttactgctccgaattgcaatatctggcgaacccggtgaccagccacgttcagtctgctgaacagcacaaccaggacgttaactcactgggtctgatcagcagccgcaaaacagccgaggcagttgacattctgaaactgatgtctactacctacctggtcgctctttgtcaggcaattgatctacgtcatctggaggaaaatctgcgccaggctgtgaaaaacactgtgtctcaagttgccaagcgtgtcttaaccacaggagcgaacggcgagctgcatccaagccgtttctgcgaaaaagatctgctcaaagtggtggaccgcgaatatgtattcgcatacgccgatgatccgtgttccgcgacgtatcccctgatgcagaaactgaggcaaattctggtagaacacgctctggcgaacggtgaaaacgagaagaacgctggcacctcggttttccagaaaatctctgcttttgaagaagaactgaaaactctactgccaaaagaagttgaatccgtccgtatcgcctacgaaagcggtaacccggcaactgctaaccgtattaaagaatgccgctcgtacccgttgtacaaattcgtgcgagaggaactgggcactggacttctgactggtgataaggtgatgtcccctggggaggagtttgacaaggtattcacggctatgtgccaggggaaaatcattgacccgatgatggactgcctgaaagaatggaacggtgctccgctgccgatatgt

SEQ ID NO: 15 - protein, 707 aa, Vitis vinifera
MNCHGSKKVESFVVSDPLNWGVAAEALKGSHLDEVKRMVAEYRKPVVRLGGETLTISQVAAIAGREADVSVELSETARAGVNASSEWVMESMSKGTDSYGVTTGFGATSHRRTKQGGALQKELIRFLNAGIFGNGTESCHTLPHSATRAAMLVRINTLLQGYSGIRFEILEAITKLLNHNITPCLPLRGTVTASGDLVPLSYIAGLLTGRPNSKAVGPSGEVVNAEEAFKMAGIESGFFELQPKEGLALVNGTAVGSGLASMVLFETNVLAVLSEVLSAIFAEVMQGKPEFTDHLTHKLKHHPGQIEAAAIMEHILDGSSYVKEAKKLHEMDPLQKPKQDRYALRTSPQWLGPQIEVIRASTKSIEREINSVNDNPLIDVSRNKALHGGNFQGTPIGVSMDNTRLAIAAIGKLMFAQFSELVNDFYNNGLPSNLSGSRNPSLDYGFKGAEIAMASYCSELQFLANPVTNHVQSAEQHNQDVNSLGLISSRKTAEAVDILKLMSTTYLVALCQAIDLRHLEENLKSTVKKTVSHVAKKTLTTGANGELHPSRFCEKALLKVVDREHVFAYIDDPCSATYPLMQKVRQVLVEHALNNGENEKNGSTSIFQKIVAFEEELKAVLPKEVESARGGVESGNPSIPNRIRECRSYPLYKFVREELGTGLLTGEKVRSPGEDFDKVFTAMCEGKIIDPLLDCLSAWNGAPLPIC

SEQ ID NO: 16 - DNA, 2121 bp, synthetic construct
  source 1..2121: mol_type = other DNA
atgaactgccatggttccaagaaagtagagagctttgtcgttagtgacccactgaattggggagtggctgccgaagcgttaaaaggctctcacctcgatgaagttaaacgtatggtagcagaatatcgcaagcccgtggttcgtctagggggtgaaacgttgaccatctctcaggtcgctgcgattgccggtcgcgaggctgacgttagcgtagaactgtcagagacagcacgagctggcgtcaacgcgtcgtccgaatgggttatggaaagcatgtctaaaggcactgatagttacggtgtgactaccggcttcggtgcaacttctcaccgtcgtaccaaacaaggcggtgctcttcagaaagaactgatacgcttcctgaacgcgggcatctttggtaacggtaccgagagctgtcatactttgccgcactccgcaacgcgtgccgctatgctggttagaattaatactttactgcaaggctactccggaatcaggttcgaaatcctggaggctatcaccaaactgctgaaccacaacatcacaccttgccttccgctgcgcggcaccgtaaccgcgtccggtgacctcgtgccgctgagctatattgcaggcctgctgactgggcgtccgaatagcaaagctgttggtccatcgggtgaagtggttaacgcagaagaggcgttcaagatggccggaatcgaatctggtttctttgaactgcaaccgaaggaaggcctggctcttgtaaacggcacggcagttggctctggtttagctagcatggtgctctttgaaactaacgtactggcagtactgtctgaagtgctgtccgccattttcgctgaagttatgcagggtaaaccggaatttaccgatcacctgacccataaactgaaacaccacccgggccagattgaggctgctgcgatcatggagcatatcctggacggctcaagttacgtcaaagaagcgaaaaaactgcacgaaatggatcctctgcaaaagccgaaacaggaccggtacgctctgcgtacttccccacagtggctgggtccgcagatagaggtgatccgtgcttctactaaaagtattgagcgcgaaatcaattcggtaaacgataacccgttaatcgacgttagccgcaacaaagctctgcatggaggtaacttccaggggacaccgatcggcgtcagcatggacaatacccgtctggccattgccgcaatcggcaagctgatgttcgcccagttttctgaactggtaaacgatttctacaacaatggtctgccatctaacctgtccggtagcagaaacccctctctcgattatgggttcaaaggcgccgaaatcgcaatggcttcgtattgctcagaattgcagtttctggcgaacccggttaccaatcacgttcagtcggctgagcagcacaaccaagatgtgaactcattgggtctgattagcagccgtaaaaccgcggaggcggttgatattctgaaactgatgtctactacgtacctcgtggctctgtgccaggcgatcgacctacgtcatctcgaagaaaaccttaagtccaccgttaaaaagactgtcagccatgttgctaagaaaaccctgacaacgggcgcaaatggcgaactgcacccgtctaggttctgtgagaaagccctgcttaaggtcgttgaccgcgaacacgtatttgcatatatcgacgatccttgctctgctacttacccactgatgcagaaagtacgtcaggtgctggtcgagcacgctctgaacaacggcgaaaacgaaaagaacggttcaacctcgatcttccagaaaatagttgcattcgaagaagagctgaaagcggtgttacctaaagaagtcgaatctgcacgtggtggagttgaaagcgggaacccgtctattcccaaccgcatccgtgaatgtcgttcctacccgctatacaagttcgtgcgtgaagaactgggcactggtcttctaaccggtgaaaaagtacgcagtcctggcgaagacttcgacaaagtttttactgccatgtgcgaggggaaaatcatcgacccgctgctggattgtctatcggcctggaacggcgcgccgctgccgatctgc

SEQ ID NO: 17 - protein, 532 aa, Photorhabdus luminescens
  source 1..532: note = subsp. laumondii
MKAKDVQPTIIINKNGLISLEDIYDIAIKQKKVEISTEITELLTHGREKLEEKLNSGEVIYGINTGFGGNANLVVPFEKIAEHQQNLLTFLSAGTGDYMSKPCIKASQFTMLLSVCKGWSATRPIVAQAIVDHINHDIVPLVPRYGSVGASGDLIPLSYIARALCGIGKVYYMGAEIDAAEAIKRAGLTPLSLKAKEGLALINGTRVMSGISAITVIKLEKLFKASISAIALAVEALLASHEHYDARIQQVKNHPGQNAVASALRNLLAGSTQVNLLSGVKEQANKACRHQEITQLNDTLQEVYSIRCAPQVLGIVPESLATARKILEREVISANDNPLIDPENGDVLHGGNFMGQYVARTMDALKLDIALIANHLHAIVALMMDNRFSRGLPNSLSPTPGMYQGFKGVQLSQTALVAAIRHDCAASGIHTLATEQYNQDIVSLGLHAAQDVLEMEQKLRNIVSMTILVVCQAIHLRGNISEIAPETAKFYHAVREISSPLITDRALDEDIIRIADAIINDQLPLPEIMLEE

SEQ ID NO: 18 - DNA, 1596 bp, synthetic construct
  source 1..1596: mol_type = other DNA
atgaaggccaaagatgtacagccaacgatcattataaataaaaacggcctgatcagcttggaggacatctatgacattgcaattaaacaaaagaaagtcgaaatctcgacagaaatcactgaactgctgactcatggtcgtgaaaaactagaggaaaaactgaactccggcgaagttatctacggtattaacaccgggttcggtggaaacgctaatctggttgtgccttttgaaaaaatcgcggaacaccagcagaacttactgaccttcctctcagctggcactggtgattacatgtctaagccgtgtatcaaagcgagccaatttaccatgctgctgtctgtatgcaaaggctggtccgccacccgccccattgttgctcaggctatcgtggaccacattaaccacgatattgtaccgcttgttccgaggtatggctctgtcggtgcaagtggtgacctgatcccgctgtcttacatcgcacgcgctctgtgcggcattggtaaagtgtactatatgggcgccgagatcgatgcggctgaagcaatcaagcgtgcaggcctgactccgttgagcttgaaagcgaaggagggtctggctctgatcaacgggacacgtgttatgagcggcatttccgcgataaccgtcatcaaactggaaaaattattcaaagcaagtatctcagctatcgcgctggctgttgaagcgctgctggccagccatgagcattacgacgcgcgtatccagcaggtcaaaaaccacccaggtcagaatgctgttgcctccgcactgagaaaccttctggctggatctactcaggttaacctcctgagtggtgtaaaagaacaggcgaataaagcatgtcgccaccaggaaattacccagctaaacgatacgctacaagaagtgtactctatacgatgcgctccgcaagtgctcgggatcgtacccgaatccctggctactgcgcgtaagattcttgagcgtgaggttatctcagctaacgacaaccctttaatcgatccggaaaacggagatgtcctgcacggtggcaacttcatgggccagtatgtagcacgtaccatggacgccctgaaactggatattgctctgattgcaaaccatctgcatgctatcgttgcgctgatgatggacaaccgcttcagccgcggtctgccgaatagcctgtctccgaccccgggcatgtaccaaggttttaagggcgtacagctgtcgcagactgcgcttgtagctgctatccgtcacgactgcgccgcaagcggtatccacaccctcgcaaccgaacagtacaatcaggatatcgtgagcttgggcctgcacgcagctcaggacgttctggaaatggaacagaagctgcgtaacatcgtgtccatgactatcttagttgtctgccaggccattcacctgagaggcaatatttctgaaattgcgccagaaacggcaaaattctaccacgctgttcgtgagatctcgtcccctctgatcacagatcgtgctctcgacgaggatatcattcgcatcgcagacgcgatcattaacgatcagttaccgctgcccgaaatcatgctggaagaa

SEQ ID NO: 19 - protein, 1090 aa, Capsicum annuum
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGSGASIAQNGHVNGDVVAIDFCKKSIHDPLNWEMAAESLKGSHLDEVKKMVDEFRKPIVKLGGETLTVAQVASIANADNKTCGAKVELSERARAGVKASSDWVMDSMCKGTDSYGVTTGFGATSHRRTKNGGALQKELIRFLNAGVFGNGTESCHTLPHSATRAAMLVRINTLLQGYSGIRFEILEAITKLINSNITPCLPLRGTITASGDLVPLSYIAGLLTGRPNSKAVGPNGEKLNAEEAFRVAGVSGGFFELQPKEGLALVNGTAVGSGMASMVLFESNILAVMSEVLSAIFAEVMNGKPEFTDHLTHKLKHHPGQIEAAAIMEHILDGSSYVKAAQKLHEMDPLQKPKQDRYALRTSPQWLGPQIEVIRAATKMIEREINSVNDNPLIDVSRNKALHGGNFQGTPIGVSMDNTRLALASIGKLMFAQFSELVNDYYNNGLPSNLTAGRNPSLDYGFKGAEIAMASYCSELQFLANPVTNHVQSAEQHNQDVNSLGLISARKTAEAVDILKLMSSTYLVALCQAIDLRHLEENLKNAVKNTVSQVAKRTLTMGANGELHPARFCEKELLRVVDREYLFAYADDPCSSTYPLMQKLRQVLVDHALNNGESEKNVNSSIFQKIAAFEDELKAVLPKEVESARITLESGNPSIPNRITECRSYPLYRLVRKELGTELLTGERVRSPGEEIDKVFTAMCNGQIIDPLLECLKSWNGAPLPIC

SEQ ID NO: 20 - DNA, 3270 bp, synthetic construct
  source 1..3270: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactggtggttctggtgcaagcattgctcagaacggtcatgtgaatggggacgttgtagccatcgatttttgtaaaaagtccatccacgatccactgaactgggaaatggctgcggagtcattgaaaggctcgcaccttgacgaagttaaaaagatggtcgacgaatttcgtaaacctatcgttaaactcggtggcgagactctgacggtagcacaagtggcttctattgcgaacgcagataataaaacctgcggtgctaaagtcgaattatctgaacgcgctcgtgcaggcgtgaaggcgagcagtgactgggttatggattctatgtgcaaagggacagactcctacggtgtaactaccggcttcggtgccacttctcaccgccgtaccaaaaacggtggcgcgctgcaaaaagaactgatacggttcctgaacgctggcgttttcggcaacggtaccgagagctgccatactctgccgcactccgctacccgtgcggcgatgctagtgagaatcaatactctcctgcaaggatattcgggtatccgtttcgaaattctggaagctattacaaagctgatcaacagcaacatcactccgtgtctgccgctgaggggcaccatcacggcctccggagacttggttcccctttcttacatcgctggtctgctgacagggcgcccgaacagcaaagccgtgggccctaacggcgaaaagctaaatgcagaggaagcgttccgagttgctggtgtatctggtggttttttcgaacttcagccgaaagagggcttagcactggttaacggcaccgctgtcggttccggtatggcatcaatggtactgttcgaatcaaacattctggctgtaatgagtgaggtgttgtctgccatctttgctgaagttatgaacggcaaaccggaatttactgatcacctcacgcataagctgaaacaccacccaggccagatcgaggcggctgcgattatggaacatatactggatggctccagctatgtcaaagcagcacagaaactgcacgaaatggaccctctgcaaaaaccgaaacaggaccgctacgcgctgcgtaccagcccgcagtggctggggccgcagatcgaagtcatccgtgccgctactaaaatgattgaacgcgaaatcaacagcgttaatgataacccgctgatcgacgtatcgcgtaacaaagcactgcatggtggtaatttccagggcactcctattggggtgtccatggacaacacccgtctggccctggcatcaattggtaaactgatgtttgcccagttcagcgaacttgttaacgattactataataacggtctgccgtctaacctgacggcgggtcgcaacccatctctcgactacggcttcaagggtgctgagatcgcaatggctagttactgctctgagctgcaattccttgcgaacccagtcaccaatcacgttcagtcagccgagcagcacaaccaggatgttaactccctgggcttgatctctgcacgcaaaacggccgaagctgtagatatcctgaaactaatgagttctacttatctggttgctttatgccaggctattgacctgcgtcatcttgaagaaaatctgaagaacgcggtaaagaacacagtgagccaggttgcaaaacgtactctaaccatgggtgccaacggcgagctgcacccggctcgtttctgtgaaaaagaactgctgcgtgttgtggatcgagagtacttattcgcctacgcggatgacccgtgcagcagcacctatccgctgatgcagaagctgcgtcaggtactggtggatcatgcactgaacaacggcgaaagcgaaaagaatgttaacagctctatctttcagaaaatcgctgctttcgaagatgagctcaaagcggtgctgcccaaagaagtggaatctgctcgcattacactggaatctgggaatccatctataccgaaccgtatcactgagtgtcgcagctacccgctttatcgtttggtgcgcaaagagctgggcactgaattgctgaccggcgaacgtgttcgctcccccggtgaagaaattgataaagtattcaccgccatgtgcaacggtcaaatcatcgacccgctgttagaatgcctgaaatcctggaacggcgctccgctgccgatctgc

(19) United States
(12) Patent Application Publication
(10) Pub. No.: US 2024/0425841 A1
(43) Pub. Date: Dec. 26, 2024

(54) ENGINEERED PHENYLALANINE AMMONIA LYASE ENZYMES

(71) Applicants: Ginkgo Bioworks, Inc., Boston, MA (US); Synlogic Operating Company, Inc., Cambridge, MA (US)

(72) Inventors: Dylan Alexander Carlin, Jamaica Plain, MA (US); Ariel Hecht, Marblehead, MA (US); Vincent Isablella, Medford, MA (US); Jonathan Lewis McMurry, Boston, MA (US); Catherine Monahan, Burlington, MA (US); Nikos Reppas, Cambridge, MA (US); Christan Ridley, Acton, MA (US); Nathan W. Schmidt, Milton, MA (US)

(73) Assignees: Ginkgo Bioworks, Inc., Boston, MA (US); Synlogic Operating Company, Inc., Cambridge, MA (US)

(21) Appl. No.: 18/690,104

(22) PCT Filed: Sep. 8, 2022
(86) PCT No.: PCT/US2022/076103

§ 371 Date: Mar. 7, 2024

Related U.S. Application Data
(60) Provisional application No. 63/241,975, filed on Sep. 8, 2021.

(51) Int. Cl. C12N 9/88; A61K 38/51; C12N 15/70; C12P 7/40

(52) U.S. Cl. CPC C12N 9/88; A61K 38/51; C12N 15/70; C12P 7/40; C12Y 403/01024

(57) ABSTRACT

Aspects of the disclosure relate to phenylalanine ammonia lyase (PAL) enzymes, including engineered PAL enzymes, and their use in catalyzing chemical reactions.

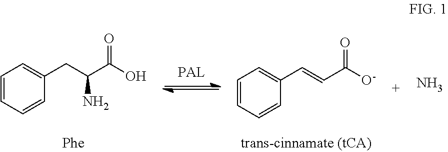

### CROSS-REFERENCE TO RELATED APPLICATIONS

This application claims the benefit under 35 U.S.C. § 119 (e) of U.S. Provisional Application No. 63/241,975, filed Sep. 8, 2021, entitled “ENGINEERED PHENYLALANINE AMMONIA LYASE ENZYMES,” the entire disclosure of which is hereby incorporated by reference in its entirety.

### REFERENCE TO AN ELECTRONIC SEQUENCE LISTING

The contents of the electronic sequence listing (G091970075WO00-SEQ-KVC.xml; Size: 46,646 bytes; and Date of Creation: Sep. 7, 2022) is herein incorporated by reference in its entirety.

### FIELD OF THE INVENTION

The present disclosure relates to the use of phenylalanine ammonia lyase enzymes, including engineered phenylalanine ammonia lyase enzymes, in the catalysis and/or bioconversion of phenylalanine to trans-cinnamic acid.

### BACKGROUND

Phenylalanine is an essential amino acid primarily found in dietary protein. Typically, a small amount is utilized for protein synthesis, and the remainder is hydroxylated to tyrosine in an enzymatic pathway that requires phenylalanine hydroxylase (PAH) and the cofactor tetrahydrobiopterin (THB). Hyperphenylalaninemia is a group of diseases associated with excess levels of phenylalanine, which can be toxic and cause brain damage. Primary hyperphenylalaninemia is caused by deficiencies in PAH activity that result from mutations in the PAH gene and/or a block in cofactor metabolism.

Phenylketonuria (PKU) is a severe form of hyperphenylalaninemia caused by mutations in the PAH gene. More than 400 different PAH gene mutations have been identified. Current PKU therapies require substantially modified diets consisting of protein restriction. Treatment from birth generally reduces brain damage, but patients must adhere rigorously to a protein-restricted diet and require supplementation of essential amino acids as well as vitamins. However, the protein-restricted diet must be carefully monitored, and essential amino acids as well as vitamins must be supplemented in the diets. Furthermore, access to low protein foods is a challenge as they are more costly than their higher protein, nonmodified counterparts.

### SUMMARY

Aspects of the present disclosure relate to a host cell that comprises a heterologous polynucleotide encoding a phenylalanine ammonia lyase (PAL) enzyme, wherein the PAL comprises a sequence that is at least 90% identical to any one of SEQ ID NOs: 1, 5, 7, 9, 11, 13 or 15, and wherein the host cell is not a plant cell. In some embodiments, the PAL comprises the sequence of any one of SEQ ID NOs: 1, 5, 7, 9, 11, 13 or 15. In some embodiments, the heterologous polynucleotide is at least 90% identical to any one of SEQ ID NOs: 2, 6, 8, 10, 12, 14 or 16. In some embodiments, the heterologous polynucleotide comprises the sequence of any one of SEQ ID NOs: 2, 6, 8, 10, 12, 14 or 16.

In some embodiments, the host cell is a bacterial cell, an archaebacterial cell, a fungal cell, a yeast cell, an animal cell, a mammalian cell, or a human cell. In some embodiments, the host cell is a bacterial cell. In some embodiments, the bacterial cell is an Escherichia coli (E. coli) cell. In some embodiments, the bacterial cell is a Bacillus cell. In some embodiments, the host cell is a filamentous fungi cell or a yeast cell. In some embodiments, the E. coli cell is an E. coli Nissle 1917 cell. In some embodiments, the PAL comprises one or more amino acid substitutions, additions, and/or deletions relative to the sequence of any one of SEQ ID NOs: 1, 5, 7, 9, 11, 13 or 15.

Other aspects of the present disclosure relate to a host cell that comprises a heterologous polynucleotide encoding a PAL enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529.

In some embodiments, the PAL comprises one or more of the following amino acid substitutions relative to SEQ ID NO: 3: I77V, F84Y, M87L, A88S, V90T, A91V, Q96E, S98A, V105F, W106Q, T110S, Y158H, Y158V, 1165V, V172A, S175G, S175T, L214Q, L219I, T243L, D253A, T345S, L364H, A394M, L406V, L407A, L407C, L407S, L407T, K413G, K413S, N415H, Q422H, 1423L, C424T, F450A, F450G, F450H, N453A, N453S, K522P and Y529L. In some embodiments, the PAL further comprises an amino acid substitution at one or more of the following amino acid residues relative to SEQ ID NO: 3: L4, T102, L104, L108, M222, G218 and N453. In some embodiments, the PAL comprises one or more of the following amino acid substitutions relative to SEQ ID NO: 3: L4P, T102E, T102K, T102R, T102S, L104A, L104M, L108A, L108H, L108Q, L108T, L108V, M222I, M222L, M222N, M222T, M222V, G218A, G218S, N453A and N453S. In some embodiments, the PAL comprises an amino acid substitution at the following amino acid residues relative to SEQ ID NO: 3: (i) L104 and L219; (ii) L4 and G218; (iii) T102 and G218; (iv) L104, 1.219 and M222; (v) T102 and G218; (vi) T102 and M222; (vii) L4, L104 and G218; (viii) G218, L104 and L219; (ix) L104, L219 and N453; or (x) G218, L4 and M222, In some embodiments, the PAL comprises any of the following relative to SEQ ID NO: 3: (i) L104M and L219I; (ii) L4P and G218S; (iii) T102K and G218A; (iv) L104M. L219I and M222L; (v) T102S and G218A; (vi) T102K and M222L; (vii) L4P, L104M and G218S; (viii) G218A, L104M and L219I; (ix) L104M, L219I and N453S; or (x) G218S, L4P and M222L. In some embodiments, the PAL comprises the following amino acid substitutions relative to SEQ ID NO: 3: L104M and L219I. In some embodiments, the PAL comprises the following amino acid substitution relative to SEQ ID NO: 3: L4P and G218S.

Other aspects of the present disclosure relate to a host cell that comprises a heterologous polynucleotide encoding a PAL enzyme, wherein the PAL comprises an amino acid substitution at one or more of the following amino acid residues relative to SEQ ID NO: 17: T40. M111. N258. N275, C288. M402. S499 and 1502. In some embodiments, the PAL comprises one or more of the following amino acid substitutions relative to SEQ ID NO: 17: T40I, M111V, N258I, N258R, N275S, C288S, M402L, S499P and I502V. In some embodiments, the PAL further comprises an amino acid substitution at one or more of the following amino acid residues relative to SEQ ID NO: 17: S92, H133, I167, A263, L432, A433 and V470. In some embodiments, the PAL further comprises one or more of the following amino acid substitutions relative to SEQ ID NO: 17: S92G, H133F, H133M, I167K, A263T, L432I, A433S and V470A. In some embodiments, the PAL comprises an amino acid substitution at the following amino acid residues relative to SEQ ID NO: 17: (i) C288, T40, I502 and S499; (ii) S92, L432, V460, H133 and N258; (iii) S92, L432, V470, N258 and T40; (iv) S92, L432, V470 and T40; (v) N258, T40, 1502 and S499; (vi) S92, V470, M111 and T40; (vii) S92, L432, V470, N258 and T40; (viii) S92, V470, A263 and T40; or (ix) S92. L432. M111 and T40. In some embodiments, the PAL comprises any one of the following relative to SEQ ID NO: 17: (i) C288S, T40I, I502V and S499P; (ii) S92G, L432I, V460A, H133F and N258I; (iii) S92G, L432I, V470A, N258I and T40I; (iv) S92G, L432I, V470A and T40I; (v) N258I, T40I, I502V and S499P; (vi) S92G, V470A, M111V and T40I; (vii) S92G, L432I, V470A, N258R and T40I; (viii) S92G, V470A, A263T and T40I; or (ix) S92G, L432I, M111V and T40I. In some embodiments, the PAL comprises the amino acid substitution N258R relative to SEQ ID NO: 17. In some embodiments, the PAL comprises the following amino acid substitutions relative to SEQ ID NO: 17: T40I, C288S, S499P and I502V.

In some embodiments, the host cell is a bacterial cell. In some embodiments, the bacterial cell is an E. coli cell. In some embodiments, the bacterial cell is a Bacillus cell. In some embodiments, the host cell is a filamentous fungi cell or a yeast cell. In some embodiments, the E. coli cell is an E. coli Nissle 1917 cell. In some embodiments, the PAL is able to convert phenylalanine to trans-cinnamic acid.

Other aspects of the present disclosure relate to methods of treating or protecting against a metabolic disorder associated with excess phenylalanine, comprising administering to a subject in need thereof a therapeutically effective amount of a PAL enzyme or a polynucleotide encoding a PAL enzyme, wherein the PAL comprises a sequence that is at least 90% identical to any one of SEQ ID NOs: 1, 5, 7, 9, 11, 13 or 15.

Other aspects of the present disclosure relates to methods of treating or protecting against a metabolic disorder associated with excess phenylalanine, comprising administering to a subject in need thereof a cell comprising a heterologous polynucleotide encoding a therapeutically effective amount of a PAL enzyme, wherein the PAL comprises a sequence that is at least 90% identical to any one of SEQ ID NOs: 1, 5, 7, 9, 11, 13 or 15. In some embodiments, the cell is a human cell, an animal cell, a yeast cell, or a bacterial cell.

Other aspects of the present disclosure relate to methods of converting phenylalanine to trans-cinnamic acid, comprising contacting phenylalanine with a PAL enzyme, wherein the PAL comprises a sequence that is at least 90% identical to any one of SEQ ID NOs: 1, 5, 7, 9, 11, 13 or 15. In some embodiments, the PAL is encoded by a polynucleotide comprising a sequence that is at least 90% identical to any one of SEQ ID NO: 2, 6, 8, 10, 12, 14 or 16. In some embodiments, the PAL comprises one or more amino acid substitutions relative to the sequence of any one of SEQ ID NOs: 1, 5, 7, 9, 11, 13 or 15.

Other aspects of the present disclosure relates to methods of treating or protecting against a metabolic disorder associated with excess phenylalanine, comprising administering to a subject in need thereof a therapeutically effective amount of a PAL enzyme or a polynucleotide encoding a PAL enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529.

Other aspects of the present disclosure relate to methods of treating or protecting against a metabolic disorder associated with excess phenylalanine, comprising administering to a subject in need thereof a cell comprising a heterologous polynucleotide encoding a therapeutically effective amount of a PAL enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529. In some embodiments, the cell is a human cell, an animal cell, a yeast cell, or a bacterial cell.

Other aspects of the present disclosure relate to methods of converting phenylalanine to trans-cinnamic acid, comprising contacting phenylalanine with a PAL enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529. In some embodiments, the PAL comprises one or more of the following amino acid substitutions relative to SEQ ID NO: 3: I77V, F84Y, M87L, A88S, V90T, A91V, Q96E, S98A, V105F, W106Q, T110S, Y158H, Y158V, I165V, V172A, S175G, S175T, L214Q, L219I, T243L, D253A, T345S, L364H, A394M, L406V, L407A, L407C, L407S, L407T, K413G, K413S, N415H, Q422H, I423L, C424T, F450A, F450G, F450H, N453A, N453S, K522P and Y529L. In some embodiments, the PAL further comprises an amino acid substitution at one or more of the following amino acid residues relative to SEQ ID NO: 3: L4, T102, L104, L108, M222, G218 and N453. In some embodiments, the PAL comprises one or more of the following amino acid substitutions relative to SEQ ID NO: 3: L4P, T102E, T102K, T102R, T102S, L104A, L104M, L108A, L108H, L108Q, L108T, L108V, M222I, M222L, M222N, M222T, M222V, G218A, G218S, N453A and N453S. In some embodiments, the PAL comprises an amino acid substitution at the following amino acid residues relative to SEQ ID NO: 3: (i) L104 and L219; (ii) L4 and G218; (iii) T102 and G218; (iv) L104, L219 and M222; (v) T102 and G218; (vi) T102 and M222; (vii) L4, L104 and G218; (viii) G218, L104 and L219; (ix) L104, L219 and N453; or (x) G218, L4 and M222, In some embodiments, the PAL comprises any of the following relative to SEQ ID NO: 3: (i) L104M and L219I; (ii) L4P and G218S; (iii) T102K and G218A; (iv) L104M, L219I and M222L; (v) T102S and G218A; (vi) T102K and M222L; (vii) L4P, L104M and G218S; (viii) G218A, L104M and L219I; (ix) L104M, L219I and N453S; or (x) G218S, L4P and M222L. In some embodiments, the PAL comprises the following amino acid substitutions relative to SEQ ID NO: 3: L104M and L219I. In some embodiments, the PAL comprises the following amino acid substitution relative to SEQ ID NO: 3: L4P and G218S.

Other aspects of the present disclosure relate to methods of treating or protecting against a metabolic disorder associated with excess phenylalanine, comprising administering to a subject in need thereof a therapeutically effective amount of a PAL enzyme or a polynucleotide encoding a PAL enzyme, wherein the PAL comprises an amino acid substitution at one or more of the following amino acid residues relative to SEQ ID NO: 17: T40, M111, N258, N275, C288, M402, S499 and I502. In some embodiments, the PAL further comprises an amino acid substitution at one or more of the following amino acid residues relative to SEQ ID NO: 17: S92, H133, 1167, A263, L432, A433 and V470.

Other aspects of the present disclosure relate to methods of treating or protecting against a metabolic disorder associated with excess phenylalanine, comprising administering to a subject in need thereof a cell comprising a heterologous polynucleotide encoding a therapeutically effective amount of a PAL enzyme, wherein the PAL comprises an amino acid substitution at one or more of the following amino acid residues relative to SEQ ID NO: 17: T40, M111, N258, N275, C288, M402, S499 and I502. In some embodiments, the PAL further comprises an amino acid substitution at one or more of the following amino acid residues relative to SEQ ID NO: 17: S92, H133, 1167, A263, L432, A433 and V470. In some embodiments, the cell is a human cell, an animal cell, a yeast cell, or a bacterial cell.

Other aspects of the present disclosure relate to methods of converting phenylalanine to trans-cinnamic acid, comprising contacting phenylalanine with a PAL enzyme, wherein the PAL comprises an amino acid substitution at one or more of the following amino acid residues relative to SEQ ID NO: 17: T40, M111, N258, N275, C288, M402, S499 and I502. In some embodiments, the PAL further comprises an amino acid substitution at one or more of the following amino acid residues relative to SEQ ID NO: 17: S92, H133, 1167, A263, L432, A433 and V470. In some embodiments, the PAL comprises one or more of the following amino acid substitutions relative to SEQ ID NO: 17: T40I, M111V, N258I, N258R, N275S, C288S, M402L, S499P and I502V. In some embodiments, the PAL further comprises one or more of the following amino acid substitutions relative to SEQ ID NO: 17: S92G, H133F, H133M, I167K, A263T, L432I, A433S and V470A. In some embodiments, the PAL comprises an amino acid substitution at the following amino acid residues relative to SEQ ID NO: 17: (i) C288, T40, I502 and S499; (ii) S92, L432, V460, H133 and N258; (iii) S92, L432, V470, N258 and T40; (iv) S92, L432, V470 and T40; (v) N258, T40, I502 and S499; (vi) 592, V470, M111 and T40; (vii) S92, L432, V470, N258 and T40; (viii) S92, V470, A263 and T40; or (ix) S92, L432, M111 and T40. In some embodiments, the PAL comprises any one of the following relative to SEQ ID NO: 17: (i) C288S, T40I, I502V and S499P; (ii) S92G, L432I, V460A, H133F and N258I; (iii) S92G, L432I, V470A, N258I and T40I; (iv) S92G, L432I, V470A and T40I; (v) N258I, T40I, I502V and S499P; (vi) S92G, V470A, M111V and T40I; (vii) S92G, L432I, V470A, N258R and T40I; (viii) S92G, V470A, A263T and T40I; or (ix) S92G, L432I, M111V and T40I. In some embodiments, the PAL comprises the amino acid substitution N258R relative to SEQ ID NO: 17. In some embodiments, the PAL comprises the following amino acid substitutions relative to SEQ ID NO: 17: T40I, C288S, S499P and I502V. In some embodiments, the method is a method of protecting a subject against phenylketonuria or hyperphenylalaninemia. In some embodiments, the method is a method of treating a subject that has phenylketonuria or hyperphenylalaninemia.

Other aspects of the present disclosure relate to a PAL enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529. In some embodiments, the PAL comprises one or more of the following amino acid substitutions relative to SEQ ID NO: 3: I77V, F84Y, M87L, A88S, V90T, A91V, Q96E, S98A, V105F, W106Q, TI10S, Y158H, Y158V, I165V, V172A, S175G, S175T, L214Q, L219I, T243L, D253A, T345S, L364H, A394M, L406V, L407A, L407C, L407S, L407T, K413G, K413S, N415H, Q422H, 1423L, C424T, F450A, F450G, F450H, N453A, N453S, K522P and Y529L. In some embodiments, the PAL further comprises an amino acid substitution at one or more of the following amino acid residues relative to SEQ ID NO: 3: L4, T102, L104, L108, M222, G218 and N453. In some embodiments, the PAL comprises one or more of the following amino acid substitutions relative to SEQ ID NO: 3: L4P, T102E, T102K, T102R, T102S, L104A, L104M, L108A, L108H, L108Q, L108T, L108V, M222I, M222L, M222N, M222T, M222V, G218A, G218S, N453A and N453S. In some embodiments, the PAL comprises an amino acid substitution at the following amino acid residues relative to SEQ ID NO: 3: (i) L104 and L219; (ii) L4 and G218; (iii) T102 and G218; (iv) L104, L219 and M222; (v) T102 and G218; (vi) T102 and M222; (vii) L4, L104 and G218; (viii) G218, L104 and L219; (ix) L104, L219 and N453; or (x) G218, L4 and M222, In some embodiments, the PAL comprises any of the following relative to SEQ ID NO: 3: (i) L104M and L219I; (ii) L4P and G218S; (iii) T102K and G218A; (iv) L104M, L219I and M222L; (v) T102S and G218A; (vi) T102K and M222L; (vii) L4P, L104M and G218S; (viii) G218A, L104M and L219I; (ix) L104M, L219I and N453S; or (x) G218S, L4P and M222L. In some embodiments, the PAL comprises the following amino acid substitutions relative to SEQ ID NO: 3: L104M and L219I. In some embodiments, the PAL comprises the following amino acid substitution relative to SEQ ID NO: 3: L4P and G218S.

Other aspects of the present disclosure relate to a PAL enzyme, wherein the PAL comprises an amino acid substitution at one or more of the following amino acid residues relative to SEQ ID NO: 17: T40, M111, N258, N275, C288, M402, S499 and I502. In some embodiments, the PAL comprises one or more of the following amino acid substitutions relative to SEQ ID NO: 17: T40I, M111V, N258I, N258R, N275S, C288S, M402L, S499P and I502V. In some embodiments, the PAL enzyme further comprises an amino acid substitution at one or more of the following amino acid residues relative to SEQ ID NO: 17: S92, H133, 1167, A263, L432, A433 and V470. In some embodiments, the PAL enzyme further comprises one or more of the following amino acid substitutions relative to SEQ ID NO: 17: S92G, H133F, H133M, I167K, A263T, L432I, A433S and V470A. In some embodiments, the PAL comprises an amino acid substitution at the following amino acid residues relative to SEQ ID NO: 17: (i) C288, T40, I502 and S499; (ii) S92, L432, V460, H133 and N258; (iii) S92, L432, V470, N258 and T40; (iv) S92, L432, V470 and T40; (v) N258, T40, I502 and S499; (vi) S92, V470, M111 and T40; (vii) S92, L432, V470, N258 and T40; (viii) S92, V470, A263 and T40; or (ix) S92, L432, M111 and T40. In some embodiments, the PAL comprises any one of the following relative to SEQ ID NO: 17: (i) C288S, T40I, I502V and S499P; (ii) S92G, L432I, V460A, H133F and N258I; (iii) S92G, L432I, V470A, N258I and T40I; (iv) S92G, L432I, V470A and T40I; (v) N258I, T40I, I502V and S499P; (vi) S92G, V470A, M111V and T40I; (vii) S92G, L432I, V470A, N258R and T40I; (viii) S92G, V470A, A263T and T40I; or (ix) S92G, L432I, M111V and T40I. In some embodiments, the PAL comprises the amino acid substitution N258R relative to SEQ ID NO: 17. In some embodiments, the PAL comprises the following amino acid substitutions relative to SEQ ID NO: 17: T40I, C288S, S499P and I502V.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIG. 1 is a schematic showing the reaction catalyzed by PAL enzymes.

FIG. 2 shows a map of the PAL expression vector used in the metagenomic analysis and the first engineered library described in Example 1.

FIG. 3 shows data from a secondary screen described in Example 1 of the PAL library in SYN107 (an E. coli Nissle 1917 (EcN) strain containing a phenylalanine importer PheP integrated into the lacZ locus under the control of a tetracycline inducible P(tet) promoter, described in and incorporated by reference from US Patent Publication No. 2017/0312320) at the 60 minute timepoint. The Y-axis shows the whole cell assay tCA (mM) concentration normalized to the OD600 of the culture at the start of the PKU potency assay. The lysate assay normalized rate is in μM tCA min-1 OD600-1. The data show the plotting of biological duplicates in technical duplicate (4 samples per PAL construct). Strain t720968 contains the P1PAL expression positive control, strain t705718 contains an intermediate PAL (YePAL) positive control and strain t722256 is a negative control comprising GFP.

FIGS. 4A-4B show two maps (FIG. 4A and FIG. 4B) of the PAL expression vectors used in the tertiary PAL screen in SYN107 described in Example 1. As described in Example 1, two ribosome binding site (RBS) strategies were employed to express the PALs on a p15a vector. In one vector, the transcriptional insulator riboJ was used upstream of selected translation initiation rates (TIRs) of ˜1e5 and ˜1e3 Arbitrary Units (AUs), with 1e5 and 1e4 associated with high gene expression and 1e3 associated with medium gene expression (FIG. 4A). In the other vector, three bicistronic designs (BCDs) were used to test different PAL expression levels. These were BCD2 for high expression, BCD12 for medium expression and BCD22 for low expression (FIG. 4B).

FIG. 5 shows data from a tertiary screen of the PAL library described in Example 1 in SYN107 at the 30 min timepoint. The Y-axis shows the whole cell assay tCA (mM) concentration normalized to the OD600 of the culture at the start of PKU potency assay. The strains were tested in biological duplicate. The P1PAL expression control strain t720968 is shown with black dots and the gray dots are library strains containing PALs on the p15a vector.

FIG. 6 shows data from a tertiary screen of the PAL library described in FIG. 5 and Example 1 in SYN107 at the 3 hour timepoint.

FIG. 7 shows data from a secondary screen described in Example 2 of the PAL engineered library. The graph shows whole cell and lysate assays without normalizing the tCA concentration and cell lysate rate data to OD600. The points on the graph represent the mean of all replicates for each strain. The error bars represent standard deviation of the mean. Strain t736486 contains the P1PAL expression positive control, strain t736484 contains an intermediate PAL (YePAL) positive control, and strain t736485 is a negative control comprising GFP.

FIG. 8 shows data from a secondary screen described in Example 2 of the PAL engineered library. The graph shows whole cell and lysate assays when the tCA concentration and cell lysate rate data are normalized to OD600. The points on the graph represent the mean of all replicates for each strain. The error bars represent standard deviation of the mean. Strain t736486 contains the P1PAL expression positive control, strain t736484 contains an intermediate PAL (YePAL) positive control, and strain t736485 is a negative control comprising GFP.

FIG. 9 shows a P1PAL homology model with the N258 and C288 amino acid residues indicated with black spheres. N258 is the black residue in the middle of the model and C288 is the black residue at the top. All other residues where mutations were identified that showed improvement in either the whole cell or cell lysate assay are shown in dark gray. Residues that were either not mutated for this screen or where mutations were not found to improve activity are shown in light gray. The Phe substrate in the active site of the depicted protein is shown as a black stick.

FIG. 10 shows the PAL expression vector used for the second engineered PAL library screen in SYN107 described in Example 2.

FIG. 11 shows secondary screen data for the second engineered P1PAL-based PAL library described in Example 2. Strains were assayed in quadruplicate (technical duplicate of biological duplicates).

FIG. 12 shows secondary screen data for the second engineered AvPAL-based PAL library described in Example 2. Strains were assayed in quadruplicate (technical duplicate of biological duplicates).

FIG. 13 shows the PAL expression vector used in the tertiary screen of the second engineered PAL library described in Example 2.

FIG. 14 shows tertiary screening results for the second P1PAL engineered library described in Example 2. The Y-axis shows the whole cell assay tCA (mM) normalized to the OD600 of the culture at the start of PKU potency assay. Strains were assayed in quadruplicate (technical duplicate of biological duplicates). The control strains, indicated with black bars, include: strain t822972 (P1PAL WT) and strain t822971 (P1PAL N258R).

FIG. 15 shows tertiary screening results for the second AvPAL engineered library described in Example 2. The Y-axis shows the whole cell assay tCA (mM) concentration normalized to the OD600 of the culture at the start of PKU potency assay. Strains were assayed in quadruplicate (technical duplicate of biological duplicates). The control strains, indicated with black bars, include strain t822970 (AvPAL WT), strain t822969 (AvPAL M222L), and strain t822968 (AvPAL L4P G218S).

FIG. 16 shows data from a follow-up screen to the second engineered library tertiary screen described in Example 2. The Y-axis shows the whole cell assay tCA (mM) concentration normalized to the OD600 of the culture at the start of PKU potency assay. Strains were assayed in quadruplicate (technical duplicate of biological duplicates). The WT control strains, indicated with black bars, include strain t822972 (P1PAL WT) and strain t822970 (AvPAL WT).

### DETAILED DESCRIPTION OF THE INVENTION

The present disclosure provides, in some aspects, engineered enzymes that are capable of enhanced phenylalanine metabolism. These enzymes include phenylalanine ammonia lyases (PALs), which are phenylalanine converting enzymes that catalyze a reaction converting L-phenylalanine to ammonia and trans-cinnamic acid. The disclosed enzymes and host cells comprising such enzymes may be used to promote phenylalanine metabolism, e.g., in a subject suffering from a disorder associated with a buildup of phenylalanine such as hyperphenylalaninemia (e.g., phenylketonuria (PKU)) and may also be used in other medical and industrial settings. The disclosure is directed, in part, to the discovery of PAL enzymes capable of degrading phenylalanine, nucleic acids encoding the same, and host cells capable of expressing PAL enzymes, e.g., in a subject having hyperphenylalaninemia (e.g., phenylketonuria (PKU)).

### Phenylalanine Ammonia Lyases (PALs)

As used in this disclosure, a “phenylalanine ammonia lyase (PAL) enzyme” refers to an enzyme that catalyzes the conversion of L-phenylalanine to ammonia and trans-cinnamic acid. In some embodiments, a PAL is a L-phenylalanine converting enzyme. Naturally occurring PALs are members of the aromatic amino acid lyase family of enzymes. Such enzymes are characterized by the presence of a co-factor (4-methyldiene-imidazol-5-one (MIO)) in their active sites, formed in naturally occurring PALs by autocatalytic cyclization and dehydration of an internal tri-peptide segment (e.g., an Ala-Ser-Gly). PALs are found in a variety of microorganisms (e.g., cyanobacteria, bacteria (e.g., actinobacteria), and extremophiles), fungi (e.g., yeast), plants, and protists (e.g., algae), but do not naturally occur in mammals. Naturally occurring PALs can have different substrate and/or product specificities; for example, PALs from dicotyledonous plants predominantly deaminate L-phenylalanine to ammonia and trans-cinnamic acid, whereas PALs from yeast and some monocot plants (e.g., maize) are known to convert L-phenylalanine and L-tyrosine to trans-cinnamic acid and p-coumaric acid, respectively. In a given plant species, multiple PAL-encoding genes may be found, increasing the number of naturally occurring PAL isoforms available for engineering. PAL enzymes occur as tetramers, with naturally occurring tetramers having molecular weights of about 64-478 kDa; heterotetramers of different naturally occurring PAL isoforms have been observed.

A PAL enzyme can use L-phenylalanine as a substrate. In some embodiments, a PAL enzyme exhibits specificity for L-phenylalanine compared to other amino acids (e.g., compared to L-tyrosine or L-histidine). In some embodiments, a PAL enzyme produces ammonia and trans-cinnamic acid from L-phenylalanine. In some embodiments, a PAL enzyme predominantly consumes L-phenylalanine relative to one or more other amino acids; e.g., a PAL enzyme may consume L-phenylalanine at a rate at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold higher (e.g., 2-fold to 6-fold more) relative to one or more other amino acids (e.g., relative to L-tyrosine or L-histidine). In some embodiments, a PAL enzyme can convert L-tyrosine into ammonia and p-coumaric acid. In some embodiments, a PAL enzyme can convert L-histidine into ammonia and urocanic acid.

In some embodiments, a PAL enzyme is capable of assembling into a multimer (e.g., in a host cell). In some embodiments, a PAL enzyme is capable of assembling into a tetramer (e.g., in a host cell). The disclosure is further directed, in part, to a fusion polypeptide comprising a plurality of PAL enzymes, wherein the plurality of PAL enzymes is capable of multimerizing, e.g., with each other. In some embodiments, the fusion polypeptide comprising a plurality of PAL enzymes comprises 2, 3, 4, 5, 6, 7, or 8 PAL enzymes or functional fragments thereof. In some embodiments, the fusion polypeptide comprises a plurality of PAL enzymes wherein each PAL enzyme comprises the same amino acid sequence or is derived from either: naturally occurring PALs from the same organism, or the same naturally occurring PAL isoform. In some embodiments, the fusion polypeptide comprises a plurality of PAL enzymes comprising a first PAL enzyme and a second PAL enzyme, wherein the amino acid sequence of the first PAL enzyme is different from the amino acid sequence of the second PAL enzyme. In some embodiments, the fusion polypeptide comprises a plurality of PAL enzymes wherein each PAL enzyme is derived from a naturally occurring PAL from a different organism, or from different naturally occurring PAL isoforms from the same organism. As used in this context, derived includes making one or more alterations to the amino acid sequence of a naturally occurring PAL (e.g., a deletion (e.g., truncation), insertion, or substitution).

In some embodiments, a PAL enzyme exhibits product inhibition, which refers to an inverse relationship between trans-cinnamic acid concentration and the rate of the PAL enzyme's production of trans-cinnamic acid and/or consumption of L-phenylalanine. In some embodiments, a PAL enzyme does not exhibit product inhibition. In some embodiments, the amino acid sequence of a PAL enzyme comprises one or more modifications relative to a corresponding wildtype sequence that alter (e.g., decrease or eliminate) product inhibition. In some embodiments, a PAL enzyme exhibits downstream product inhibition, which refers to an inverse relationship between a downstream product concentration and the rate of the PAL enzyme's production of trans-cinnamic acid and/or consumption of L-phenylalanine. In some embodiments, a downstream product is any compound produced by an enzyme downstream of PAL in a metabolic pathway. The downstream product may be produced by said metabolic pathway in a non-host cell (e.g., a cell comprising a naturally occurring PAL from which a PAL enzyme was derived), but the downstream product may be present in a host cell regardless of the presence of the metabolic pathway in the host cell. In some embodiments, a downstream product includes, but is not limited to: p-coumarate, p-coumaroyl CoA, a stilbene, an isoflavonoid, a flavonol, a flavonol glycoside, caffeic acid, ferulic acid, sinapic acid, or a monolignol (e.g., p-coumaryl alcohol, coniferyl alcohol, or sinapyl alcohol). In some embodiments, a PAL enzyme does not exhibit downstream product inhibition. In some embodiments, the amino acid sequence of a PAL enzyme comprises one or more modifications relative to a corresponding wildtype sequence that alter (e.g., decrease or eliminate) downstream product inhibition.

In some embodiments, a PAL enzyme capable of assembling into a multimer exhibits negative cooperativity with respect to binding and/or catalyzing conversion of L-phenylalanine. In some embodiments, a PAL enzyme capable of assembling into a multimer does not exhibit negative cooperativity with respect to binding and/or catalyzing conversion of L-phenylalanine. In some embodiments, the amino acid sequence of a PAL enzyme comprises one or more modifications relative to a corresponding wildtype sequence that alter (e.g., decrease or eliminate) negative cooperativity. In some embodiments, a fusion polypeptide comprising a plurality of PAL enzymes comprises PAL enzymes that do not exhibit negative cooperativity with respect to binding and/or catalyzing conversion of L-phenylalanine.

In some embodiments, a host cell comprises a PAL enzyme from Photorhabdus laumondii (P1PAL), Gossypium raimondii (GrPAL), Anabaena variabilis (AvPAL), Rhizobium radiobacter (RrPAL), Arabidopsis thaliana (AtPAL2), Capsicum annuum (CaPAL), Salvia miltiorrhiza (SmPAL), Ricinus communis (RcPAL), or Vitis vinifera (VvPAL). In some embodiments, a host cell comprises a PAL enzyme fused with a maltose-binding protein (MBP), e.g., MBP-CaPAL. In some embodiments, a host cell comprises a PAL enzyme from algae, e.g., from Dunaliella marina. In some embodiments, a host cell comprises a PAL enzyme from Ascomycota (e.g., Nectria cinnabarina), Basidiomycota (e.g., Ustilago maydis, Rhodotorula rubra, R. graminis, R. glutinis, or Rhodosporidium toruloides), cyanobacteria (e.g., Anabaena variabilis, Nostoc punctiforme, Synechocystis sp., Oscillatoria sp., or Leptolyngbya sp.) or bacteria (e.g., Streptomyces maritimus, Streptomyces verticillatus, Rhodobacter capsulatus or Photorhabdus luminescens). In some embodiments, a host cell comprises a PAL from a microorganism, such as Brevibacillus laterosporus (B1PAL), Dictyostelium discoideum (DdPAL), Streptomyces rimosus (SrPAL), Planctomyces brasiliensis (PbPAL), or a Methylobacterium species (MxPAL). In some embodiments, a host cell comprises a PAL from an extremophile, such as Rubrobacter xylanophilus (RxPAL), Pseudozyma antarctica (PzaPAL), or Kangiella koreensis (KkPAL). In some embodiments, a host cell comprises a PAL enzyme from a species, genus, or family described within this disclosure and comprises one or more substitutions that replace an amino acid present in the wildtype amino acid sequence with an amino acid present in a different PAL enzyme (e.g., a PAL enzyme from a different species, genus, or family, or different isoform from the same species). In some embodiments, a host cell comprises a PAL enzyme from a species, genus, or family described within this disclosure and comprises one or more substitutions, insertions, or deletions at positions recited within this disclosure or positions corresponding thereto. In some embodiments, a host cell comprises a functional fragment of a PAL enzyme described within this disclosure. Functional and structural characterization of several naturally occurring PAL proteins can be found, for example, in Kawatra et al. Biochimie. 2020 October; 177:142-152, which is incorporated by reference in its entirety within this disclosure.

In some embodiments, a host cell comprises a PAL enzyme and/or a heterologous polynucleotide encoding such an enzyme. In some embodiments, a host cell comprises a heterologous polynucleotide encoding a PAL enzyme comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any one of SEQ ID NO: 1, 3, 5, 7, 9, 11, 13, 15, or 17, a PAL enzyme in Table 3 or Table 5, or a PAL enzyme otherwise described in this disclosure. In some embodiments, a host cell comprises a heterologous polynucleotide that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any one of SEQ ID NO: 2, 4, 6, 8, 10, 12, 14, 16, or 18, a polynucleotide encoding a PAL enzyme in Table 3 or Table 5, or a PAL enzyme otherwise described in this disclosure.

In some embodiments, the PAL enzyme is a Gossypium raimondii (New World cotton) PAL or GrPAL. The Gossypium raimondii PAL is provided by SEQ ID NO: 1, which corresponds to the sequence provided by UniProtKB Accession No. A0A0D2QBF2 (expressed in strain t727023 described in the Examples):

METITQNGHHKGSLESFCTASKGAGGAVDPLNWGVAAESLKGSHLDEVK
RMVAEYRNPLVKLGGETLTISQVAAIATRDLGVKVELSEDARAGVKASA
DWVLDGMNKGTDSYGVTTGFGATSHRRTKQGAALQKELIRFLNAGVFGH
GTESCHTLPHSATRAAMLVRINTLLQGYSGIRFEILEAITKLINSNITP
CLPLRGTITASGDLVPLSYIAGLLTGRPNSKAVGPNGEPLDAEEAFRVA
GIDSGFFVLQPKEGLALVNGTAVGSGMASMVLFEANILAVLSEVLSAIF
AEVMNGKPEFTDHLTHKLKHHPGQIEAAAIMEHILEGSSYVKAAKKLHE
MDPLQKPKQDRYALRTSPQWLGPQIEVIRFATKSIEREINSVNDNPLID
VSRNKALHGGNFQGTPIGVSMDNARLAIAAIGKLMFAQFSELVNDFYNN
GLPSNLSGGRNPSLDYGFKGAEIAMASYCSELQYLANPVTSHVQSAEQH
NQDVNSLGLISSRKTAEAVDILKLMSSTYLAALCQAIDLRHLEENLRST
VKNTVSQIAKKVLTTGANGELHPSRFCEKDLLKAVDREYVFAYIDDPCS
ATYPLMQKLRQVLVEHALINGENEKNASTSIFQKIAAFEEELKAVLPKE
VESARASVENGNAAIPNKIKECRSFPLYKFVREELGIGLLTGENVMSPG
EEFDKVFTAMCQGRIIDPMLECLEEWNGAPLPIC

A non-limiting example of a nucleotide sequence encoding SEQ ID NO: 1 is provided by SEQ ID NO: 2:

atggaaaccattacgcaaaatggccaccataaaggttccctagagtcgt
tttgtacagcaagcaagggggccggcggtgcggtcgatcctttgaactg
gggtgtagctgctgaatctttaaaaggctcacacctggacgaagttaaa
cgtatggttgcagagtatagaaacccgctggtgaagcttgggggagaaa
ctctcaccatctctcaggtggcggctatcgctacccgcgatctgggtgt
aaaagttgaactgagcgaggacgctcgtgcgggcgtaaaagcatccgct
gattgggttctggacggtatgaacaaaggcactgactcttacggtgtca
ccactggtttcggcgcgactagccatcgacgcacaaaacagggcgccgc
cctgcaaaaggaactgatacgcttcctgaacgcgggagtgtttggtcac
ggcaccgaaagttgccacaccctgccccattcggcaactcgtgctgcta
tgctggtccgtatcaatacgctcctgcaaggttactctggcattcgttt
cgaaatcctggaagctattactaaactcatcaacagcaacattaccccg
tgcttgccgctgcgcggtacgatcactgcgtctggcgatctggttccat
tatcctatatcgcaggcctgctgacgggtcgtccgaacagcaaggccgt
tggccctaacggtgagccgctggacgctgaagaggcattccgtgtagcc
gggattgattctggatttttcgttctgcaaccaaaagaaggcctggcgc
tggtgaatggtaccgctgttggttccggcatggctagcatggtgctgtt
tgaagctaacatcctggcggtgctgtctgaggtactgagcgcaatcttc
gcagaagttatgaacggtaaaccggagttcacagaccaccttactcata
aacttaaacaccatccggggcagatcgaagcggcagcgattatggaaca
catactggaaggtagttcctacgtaaaagctgcaaaaaagctacacgaa
atggatccattgcagaaaccgaaacaggaccgctacgccctccgcacct
cgccgcagtggctgggcccgcagattgaggtaatccgtttcgctaccaa
atctatcgaaagggaaatcaactctgttaacgacaacccgttaatcgat
gtctctcgtaacaaggcactgcacggcggtaattttcagggaactccaa
ttggtgtttctatggataacgctcggcttgctattgctgccatcggtaa
actgatgttcgcccagttctccgagctggtaaacgatttctataacaat
ggcttgccgagcaatctgagcggcgggaggaacccgtctttggactacg
gtttcaaaggtgcagaaatcgcgatggctagttattgcagtgaactgca
atacctggctaatccggtaacctcccacgtgcagtccgcagagcagcac
aaccaggacgtgaacagcctgggcctgatcagctcccgcaaaactgcgg
aagctgttgatattctgaagctgatgtcgtcaacttacttagcggcgct
gtgtcaggcaatcgacctgcgtcatctggaagaaaacctgcgttctacc
gttaaaaacactgtttcgcagattgctaaaaaggttctgacgaccggtg
cgaatggcgaactccacccttctcgtttttgcgaaaaagacctgctaaa
agccgtggatcgtgaatatgtctttgcgtacatcgacgatccgtgtagc
gctacgtacccactgatgcagaaactgcgccaagtgctggttgaacatg
cactgaccaacggggaaaacgagaaaaacgctagcaccagtatcttcca
gaagatagcggcattcgaggaagagctgaaagcggttcttcccaaagag
gttgaatcggcgcgtgcgtccgtagaaaatggaaacgctgccattccga
acaaaatcaaagaatgccgctcttttccgttatacaaatttgtgcgtga
agagctgggcactggtttgctgacgggcgagaacgttatgtcccccggt
gaagaatttgacaaggtcttcaccgctatgtgccaggggcgtatcatcg
atccaatgttagaatgcctggaggaatggaacggcgcccctctaccgat
ttgc

In some embodiments, the PAL enzyme is an Anabaena variabilis PAL or AvPAL. The Anabaena variabilis PAL is provided by SEQ ID NO: 3, which corresponds to the sequence provided by UniProtKB Accession No. Q3M5Z3 (expressed in strain t726062 described in the Examples):

MKTLSQAQSKTSSQQFSFTGNSSANVIIGNQKLTINDVARVARNGTLVS
LTNNTDILQGIQASCDYINNAVESGEPIYGVTSGFGGMANVAISREQAS
ELQTNLVWFLKTGAGNKLPLADVRAAMLLRANSHMRGASGIRLELIKRM
EIFLNAGVTPYVYEFGSIGASGDLVPLSYITGSLIGLDPSFKVDFNGKE
MDAPTALRQLNLSPLTLLPKEGLAMMNGTSVMTGIAANCVYDTQILTAI
AMGVHALDIQALNGTNQSFHPFIHNSKPHPGQLWAADQMISLLANSQLV
RDELDGKHDYRDHELIQDRYSLRCLPQYLGPIVDGISQIAKQIEIEINS
VTDNPLIDVDNQASYHGGNFLGQYVGMGMDHLRYYIGLLAKHLDVQIAL
LASPEFSNGLPPSLLGNRERKVNMGLKGLQICGNSIMPLLTFYGNSIAD
RFPTHAEQFNQNINSQGYTSATLARRSVDIFQNYVAIALMFGVQAVDLR
TYKKTGHYDARACLSPATERLYSAVRHVVGQKPTSDRPYIWNDNEQGLD
EHIARISADIAAGGVIVQAVQDILPCLH

A non-limiting example of a nucleotide sequence encoding SEQ ID NO: 3 is provided by SEQ ID NO: 4:

atgaaaacactgtcccaagcacagtcgaagacgagtagccagcagtttt
ctttcaccggtaattctagcgctaacgtgattatcggcaaccagaaact
aactatcaacgacgtcgcccgcgttgctcggaacgggaccttagtaagc
ctgactaacaacactgatatacttcaaggcattcaggcgtcctgtgatt
atatcaataacgcagttgagtctggtgaaccaatttacggtgtgacctc
aggcttcggcggtatggcgaacgttgctatcagccgtgaacaggcctcc
gaactgcaaaccaatttggtatggtttctgaaaactggtgctgggaaca
aactccctttggcggacgtacgtgcagcaatgctgctgcgcgccaactc
gcatatgcgtggcgcgtccggtatccgtctggagctgatcaaacgtatg
gaaatcttcttgaacgctggtgttactccgtacgtttatgaatttggat
ctatcggcgcttctggagatctggtcccgctgagctacattacgggttc
cctgattggccttgacccgagcttcaaggtggatttcaacggcaaagaa
atggacgccccgaccgcgttacgccagttaaatctgtctcccctgacac
ttctgcctaaagaaggtctagctatgatgaatgggacctcagtcatgac
tggcatcgcagctaactgcgtatacgacacccagatcctgactgcgatt
gcaatgggtgttcacgctctggatatccaggccctgaacggcaccaacc
agtctttccacccgtttatccataactctaagccgcacccaggtcagct
gtgggcggctgatcagatgatatcattgctggctaactcgcaactggta
cgggacgagctggacggcaaacatgattaccgcgaccacgagctgatcc
aggatcgttatagcctgcgttgccttccgcagtacctgggtccgattgt
ggacggtatctcacagatagcaaaacaaatcgaaattgaaattaactcc
gttactgataaccctctgattgacgtcgataaccaggcgtcgtaccacg
gcggaaatttcctgggtcagtatgttggcatgggtatggaccaccttcg
ctactatatcggcctgctggcgaaacacctggatgtgcagattgcgctg
ctagctagtcccgaatttagcaacggactgccgccatctttattgggca
accgtgaacgtaaggttaacatgggtctgaaaggtttacaaatctgtgg
caattccatcatgccgctgctgacgttctacggcaatagcatcgccgac
cgctttccgacccatgcagagcaattcaaccagaatatcaactctcagg
gctacacctccgcaacgctggcgcgacgtagtgttgatatcttccaaaa
ctacgttgcgattgccctgatgtttggcgtccaggctgtagacctgagg
acttataaaaagactggccattacgatgcgcgtgcttgcctctctccgg
ctaccgaacgcctgtattccgccgtgcgtcacgtagttggtcagaaacc
tacttcagatcgcccatacatctggaacgataacgagcagggtctggat
gaacacatcgctcgcatctccgctgacattgccgctggcggagtaattg
ttcaagctgtacaggatatcctgccgtgcctgcac

In some embodiments, the PAL enzyme is a Rhizobium radiobacter PAL or RrPAL. The Rhizobium radiobacter PAL is provided by SEQ ID NO: 5, which corresponds to the sequence provided by UniProtKB Accession No. AOA1B9UCP2 (expressed in strain t726692 described in the Examples):

MTVLLDDGLSWRDVAHIGKGEALRVSDAAYLRIDRASQIVDSIVESGVR
AYGINTGVGALSDTVVDRTAQGRLSRSIILSHACGVGRLLDPREVRAIM
AAQIANFAHGHSGVRREIVSHLSTFLEQDCIPDVPSRGSAGYLVHNAHV
ALVLIGEGQARLGGRVMSGRDALAAMGLQPIVLGAKEGLSLVNGTACAT
GLSCMALARANHLLDWADAIAALTLEAAGCQIDAFDKTVLALRPSKGIA
AVGAALRSRLEGSGLVAAAHGRRTQDALSLRAVPHAHGAARDIFDACAS
IVDRELASVTDNPAILGTPEQPIVSSEAHAVAPALGQAADSLAIAIAQI
ASMSERRIDRLVNPLVSGLPPFLATDAGSHSGFMIAQYTAAALVGDSRR
LSAPASTDGGLTSGLQEDFLSHPTAAANKLLAVLDNAEYILAIEWMAGV
QAHDSLESVAGRAAGTNVVYNLLREHLQPYSDDRPLSADMEKARLLLRD
LSPPDM

A non-limiting example of a nucleotide sequence encoding SEQ ID NO: 5 is provided by SEQ ID NO: 6:

atgaccgttctcttggatgacggtctgtcttggcgggacgtggcgcatattggcaaaggtgaagcact
tcgcgtaagcgatgctgcctatctgcgtatcgatcgtgctagtcaaatcgtcgactccattgttgagt
caggcgtacgcgcatacgggataaatacgggtgtgggcgctctgtcggacactgttgttgatcgtaca
gcgcagggaagattatctcgttccatcatcctgagccacgcgtgcggtgtcggccgcctgctggaccc
gagggaagtgcgtgctattatggcagctcagatcgccaactttgcacacggtcactctggcgttcgtc
gtgaaatcgtaagtcatctgagcactttcctggaacaggattgtatcccagacgttccctcccgcggc
tctgctggctacctggttcataacgcacacgtcgcgttggtactgattggtgagggtcaggcccgtct
gggtggtcgcgttatgagcgggcgtgatgccctggctgcgatggggctccagccgatagtgctgggcg
cgaaggaaggactgtcgttagtaaacggtaccgcctgcgctaccggcttatcttgcatggcgctggct
cgagcaaaccacctgttggactgggctgatgctatcgctgcactgactctggaagcagcgggctgtca
aatcgacgcattcgataaaacggttctggcgctgcgcccgagcaaaggtattgctgctgttggcgcag
ccctccgttcacgtctggagggttccggactggtggcggctgcacacggccgccgtactcaggacgcg
ctgtccctgcgcgctgttcctcatgcacacggtgcagcgcgcgatatctttgacgcatgcgcttctat
cgtcgatcgtgaactggcgagcgtgaccgacaatccggcaattcttggcactccggagcagcctatcg
tatcttccgaagctcacgctgttgccccggctctgggtcaggctgccgacagcctagcgattgcgatt
gcacaaatcgccagcatgtctgaaaggcgtatcgatcggctagttaacccgctggtcagcggacttcc
gccattcttggcgaccgatgctggtagccactcaggcttcatgattgcgcagtatactgctgcggctc
tggtaggcgattcgcgccgtctgtcggcccctgcgtctactgacggtggcctgacctccggtcttcaa
gaagactttctgtctcatccgaccgccgctgcaaacaaacttctagcggtactggacaatgctgaata
catcctggccatcgagtggatggctggtgttcaggcgcacgatagtctggagtccgtagcggggcgcg
cggctggcacgaacgtggtatacaaccttttgcgtgaacacctacagccatatagtgacgatcgaccg
ctgtctgccgatatggaaaaggcgcgtctgctgctgcgtgacctgtcccctccggacatg

In some embodiments, the PAL enzyme is an Arabidopsis thaliana PAL or AtPAL2. The Arabidopsis thaliana PAL is provided by SEQ ID NO: 7, which corresponds to the sequence provided by UniProtKB Accession No. P45724 (expressed in strain t731343 described in the Examples):

MDQIEAMLCGGGEKTKVAVTTKTLADPLNWGLAADQMKGSHLDEVKKMVEEYRRPVVNLGGETLTIGQ
VAAISTVGGSVKVELAETSRAGVKASSDWVMESMNKGTDSYGVTTGFGATSHRRTKNGTALQTELIRF
LNAGIFGNTKETCHTLPQSATRAAMLVRVNTLLQGYSGIRFEILEAITSLLNHNISPSLPLRGTITAS
GDLVPLSYIAGLLIGRPNSKATGPDGESLTAKEAFEKAGISTGFFDLQPKEGLALVNGTAVGSGMASM
VLFEANVQAVLAEVLSAIFAEVMSGKPEFTDHLTHRLKHHPGQIEAAAIMEHILDGSSYMKLAQKVHE
MDPLQKPKQDRYALRTSPQWLGPQIEVIRQATKSIEREINSVNDNPLIDVSRNKAIHGGNFQGTPIGV
SMDNTRLAIAAIGKLMFAQFSELVNDFYNNGLPSNLTASSNPSLDYGFKGAEIAMASYCSELQYLANP
VTSHVQSAEQHNQDVNSLGLISSRKTSEAVDILKLMSTTFLVGICQAVDLRHLEENLRQTVKNTVSQV
AKKVLITGINGELHPSRFCEKDLLKVVDREQVFTYVDDPCSATYPLMQRLRQVIVDHALSNGETEKNA
VISIFQKIGAFEEELKAVLPKEVEAARAAYGNGTAPIPNRIKECRSYPLYRFVREELGTKLLTGEKVV
SPGEEFDKVFTAMCEGKLIDPLMDCLKEWNGAPIPIC

A non-limiting example of a nucleotide sequence encoding SEQ ID NO: 7 is provided by SEQ ID NO: 8:

atggaccaaatcgaggctatgctgtgtgggggaggcgaaaaaacaaaggttgcagtaacgactaaaac
cttagccgatcctcttaactggggtctggctgcggaccagatgaaaggttctcatttggatgaagtga
agaaaatggtcgaagaatatcgccgtcccgttgttaatctaggcggcgagactctgaccattggtcag
gtggcagcgatcagcaccgtaggcggttcagttaaagttgaactcgctgaaacttcgcgtgctggtgt
gaaagcatccagcgattgggtgatggagtctatgaacaaaggcactgactcctacggagtaaccacgg
gttttggcgcgacttctcaccgccgtaccaagaacggtaccgctctgcaaacagaactgatacgattc
ctgaatgccggtatcttcggcaacactaaagagacttgccacaccctgccacagagcgcgactcgtgc
tgctatgctggttagggtgaacaccctgctgcaagggtacagtggcattcgtttcgaaatcctggaag
caattacatcgctgctgaaccataatatctcaccgagcttgccgctgcgcggtacgatcaccgcatcc
ggtgacttagtcccgctgtcttatatcgcagggctgttgacgggtcgcccgaactccaaagccactgg
cccggatggcgagagcctgaccgctaaagaagcgtttgaaaaggccggcatctctactggtttcttcg
acctgcaaccgaaagaaggtcttgcactggtaaacggcaccgccgtaggcagtggtatggcatctatg
gtgctgtttgaagctaacgttcaggctgttctggcggaagttttatccgcgattttcgctgaggtgat
gagcggtaaaccggaatttacggatcacctcacccaccgcttaaaacatcaccctggccagattgaag
ccgctgcaatcatggaacacattctcgacggttcttcctacatgaaattggctcagaaagtccatgag
atggacccactgcaaaaaccaaaacaggatcgttacgcgctgcgtacttctccgcagtggctaggacc
gcagatcgaagttatccgtcaggctaccaagtccattgaacgagaaattaacagcgtcaacgacaacc
ccctgattgatgtatcgcgcaacaaggcaatccacggcggtaacttccaggggacaccgatcggcgtt
agcatggataatactcgtttagccatcgcagcaatcggtaagctgatgtttgctcagttctctgagct
ggttaatgacttttataacaacgggcttccttcgaatctcaccgctagctccaacccgtctttagact
acggtttcaaaggcgctgaaatcgcgatggcgtcctattgctcagaactgcaatacctggcgaacccg
gtgacctcacatgtgcagagcgctgagcagcacaaccaggatgtcaactctctgggcctgataagttc
aaggaaaacttcagaagctgtcgacatccttaagctcatgtctacgaccttcctggtaggcatctgcc
aggctgttgatctgcgtcacctggaggaaaacttacgtcagactgttaaaaacactgtaagccaagtc
gctaaaaaagttctgacgaccggcatcaacggcgagctgcacccttcccgcttctgcgagaaggatct
actgaaagttgtggaccgtgaacaggttttcacttacgttgacgatccgtgttctgcgacttacccat
tgatgcagcggctgcgccaggtgatagttgatcatgcactgagcaatggtgaaaccgaaaagaacgct
gttactagcatttttcaaaaaatcggtgctttcgaggaagagctgaaagcggtactgccgaaagaagt
ggaagccgcgcgtgctgcgtatggcaacggtaccgcaccgatcccgaaccgtatcaaggaatgccgta
gttatccactgtaccgcttcgtacgtgaagaactgggtaccaagctgttaaccggggaaaaagttgtt
tctcctggcgaagaatttgacaaagtatttacggccatgtgcgaaggcaaactgattgacccgctgat
ggattgcctaaaagaatggaacggtgctcccatcccgatttgt

In some embodiments, the PAL enzyme is a Capsicum annuum (Capsicum pepper) PAL or CaPAL. The Capsicum annuum PAL is provided by SEQ ID NO: 9, which corresponds to the sequence provided by UniProtKB Accession No. A0A1U8E697:

MASIAQNGHVNGDVVAIDFCKKSIHDPLNWEMAAESLKGSHLDEVKKMVDEFRKPIVKLGGETLTVAQ
VASIANADNKTCGAKVELSERARAGVKASSDWVMDSMCKGTDSYGVTTGFGATSHRRTKNGGALQKEL
IRFLNAGVFGNGTESCHTLPHSATRAAMLVRINTLLQGYSGIRFEILEAITKLINSNITPCLPLRGTI
TASGDLVPLSYIAGLLTGRPNSKAVGPNGEKLNAEEAFRVAGVSGGFFELQPKEGLALVNGTAVGSGM
ASMVLFESNILAVMSEVLSAIFAEVMNGKPEFTDHLTHKLKHHPGQIEAAAIMEHILDGSSYVKAAQK
LHEMDPLQKPKQDRYALRTSPQWLGPQIEVIRAATKMIEREINSVNDNPLIDVSRNKALHGGNFQGTP
IGVSMDNTRLALASIGKLMFAQFSELVNDYYNNGLPSNLTAGRNPSLDYGFKGAEIAMASYCSELQFL
ANPVTNHVQSAEQHNQDVNSLGLISARKTAEAVDILKLMSSTYLVALCQAIDLRHLEENLKNAVKNIV
SQVAKRTLTMGANGELHPARFCEKELLRVVDREYLFAYADDPCSSTYPLMQKLRQVLVDHALNNGESE
KNVNSSIFQKIAAFEDELKAVLPKEVESARITLESGNPSIPNRITECRSYPLYRLVRKELGTELLTGE
RVRSPGEEIDKVFTAMCNGQIIDPLLECLKSWNGAPLPIC

A non-limiting example of a nucleotide sequence encoding SEQ ID NO: 9 is provided by SEQ ID NO: 10:

atggcaagcattgctcagaacggtcatgtgaatggggacgttgtagccatcgatttttgtaaaaagtc
catccacgatccactgaactgggaaatggctgcggagtcattgaaaggctcgcaccttgacgaagtta
aaaagatggtcgacgaatttcgtaaacctatcgttaaactcggtggcgagactctgacggtagcacaa
gtggcttctattgcgaacgcagataataaaacctgcggtgctaaagtcgaattatctgaacgcgctcg
tgcaggcgtgaaggcgagcagtgactgggttatggattctatgtgcaaagggacagactcctacggtg
taactaccggcttcggtgccacttctcaccgccgtaccaaaaacggtggcgcgctgcaaaaagaactg
atacggttcctgaacgctggcgttttcggcaacggtaccgagagctgccatactctgccgcactccgc
tacccgtgcggcgatgctagtgagaatcaatactctcctgcaaggatattcgggtatccgtttcgaaa
ttctggaagctattacaaagctgatcaacagcaacatcactccgtgtctgccgctgaggggcaccatc
acggcctccggagacttggttcccctttcttacatcgctggtctgctgacagggcgcccgaacagcaa
agccgtgggccctaacggcgaaaagctaaatgcagaggaagcgttccgagttgctggtgtatctggtg
gttttttcgaacttcagccgaaagagggcttagcactggttaacggcaccgctgtcggttccggtatg
gcatcaatggtactgttcgaatcaaacattctggctgtaatgagtgaggtgttgtctgccatctttgc
tgaagttatgaacggcaaaccggaatttactgatcacctcacgcataagctgaaacaccacccaggcc
agatcgaggcggctgcgattatggaacatatactggatggctccagctatgtcaaagcagcacagaaa
ctgcacgaaatggaccctctgcaaaaaccgaaacaggaccgctacgcgctgcgtaccagcccgcagtg
gctggggccgcagatcgaagtcatccgtgccgctactaaaatgattgaacgcgaaatcaacagcgtta
atgataacccgctgatcgacgtatcgcgtaacaaagcactgcatggtggtaatttccagggcactcct
attggggtgtccatggacaacacccgtctggccctggcatcaattggtaaactgatgtttgcccagtt
cagcgaacttgttaacgattactataataacggtctgccgtctaacctgacggcgggtcgcaacccat
ctctcgactacggcttcaagggtgctgagatcgcaatggctagttactgctctgagctgcaattcctt
gcgaacccagtcaccaatcacgttcagtcagccgagcagcacaaccaggatgttaactccctgggctt
gatctctgcacgcaaaacggccgaagctgtagatatcctgaaactaatgagttctacttatctggttg
ctttatgccaggctattgacctgcgtcatcttgaagaaaatctgaagaacgcggtaaagaacacagtg
agccaggttgcaaaacgtactctaaccatgggtgccaacggcgagctgcacccggctcgtttctgtga
aaaagaactgctgcgtgttgtggatcgagagtacttattcgcctacgcggatgacccgtgcagcagca
cctatccgctgatgcagaagctgcgtcaggtactggtggatcatgcactgaacaacggcgaaagcgaa
aagaatgttaacagctctatctttcagaaaatcgctgctttcgaagatgagctcaaagcggtgctgcc
caaagaagtggaatctgctcgcattacactggaatctgggaatccatctataccgaaccgtatcactg
agtgtcgcagctacccgctttatcgtttggtgcgcaaagagctgggcactgaattgctgaccggcgaa
cgtgttcgctcccccggtgaagaaattgataaagtattcaccgccatgtgcaacggtcaaatcatcga
cccgctgttagaatgcctgaaatcctggaacggcgctccgctgccgatctgc

In some embodiments, the PAL enzyme is a Capsicum annuum (Capsicum pepper) PAL or CaPAL fused with a maltose-binding protein (MBP-CaPAL). The Capsicum annuum MBP-CaPAL fusion protein is provided by SEQ ID NO: 19 (expressed in strain t732438 described in the Examples):

MKIEEGKLVIWINGDKGYNGLAEVGKKFEKDTGIKVTVEHPDKLEEKFPQVAATGDGPDIIFWAHDRF
GGYAQSGLLAEITPDKAFQDKLYPFTWDAVRYNGKLIAYPIAVEALSLIYNKDLLPNPPKTWEEIPAL
DKELKAKGKSALMENLQEPYFTWPLIAADGGYAFKYENGKYDIKDVGVDNAGAKAGLTFLVDLIKNKH
MNADTDYSIAEAAFNKGETAMIINGPWAWSNIDTSKVNYGVTVLPTFKGQPSKPFVGVLSAGINAASP
NKELAKEFLENYLLTDEGLEAVNKDKPLGAVALKSYEEELAKDPRIAATMENAQKGEIMPNIPQMSAF
WYAVRTAVINAASGRQTVDEALKDAQTGGSGASIAQNGHVNGDVVAIDFCKKSIHDPLNWEMAAESLK
GSHLDEVKKMVDEFRKPIVKLGGETLTVAQVASIANADNKTCGAKVELSERARAGVKASSDWVMDSMC
KGTDSYGVTTGFGATSHRRTKNGGALQKELIRFLNAGVFGNGTESCHTLPHSATRAAMLVRINTLLQG
YSGIRFEILEAITKLINSNITPCLPLRGTITASGDLVPLSYIAGLLTGRPNSKAVGPNGEKLNAEEAF
RVAGVSGGFFELQPKEGLALVNGTAVGSGMASMVLFESNILAVMSEVLSAIFAEVMNGKPEFTDHLTH
KLKHHPGQIEAAAIMEHILDGSSYVKAAQKLHEMDPLQKPKQDRYALRTSPQWLGPQIEVIRAATKMI
EREINSVNDNPLIDVSRNKALHGGNFQGTPIGVSMDNTRLALASIGKLMFAQFSELVNDYYNNGLPSN
LTAGRNPSLDYGFKGAEIAMASYCSELQFLANPVTNHVQSAEQHNQDVNSLGLISARKTAEAVDILKL
MSSTYLVALCQAIDLRHLEENLKNAVKNTVSQVAKRTLIMGANGELHPARFCEKELLRVVDREYLFAY
ADDPCSSTYPLMQKLRQVLVDHALNNGESEKNVNSSIFQKIAAFEDELKAVLPKEVESARITLESGNP
SIPNRITECRSYPLYRLVRKELGTELLTGERVRSPGEEIDKVFTAMCNGQIIDPLLECLKSWNGAPLP
IC

A non-limiting example of a nucleotide sequence encoding SEQ ID NO: 19 is provided by SEQ ID NO: 20:

atgaaaatcgaagaaggtaaactggtaatctggattaacggcgataaaggctataacggtctcgctga
agtcggtaagaaattcgagaaagataccggaattaaagtcaccgttgagcatccggataaactggaag
agaaattcccacaggttgcggcaactggcgatggccctgacattatcttctgggcacacgaccgcttt
ggtggctacgctcaatctggcctgttggctgaaatcaccccggacaaagcgttccaggacaagctgta
tccgtttacctgggatgccgtacgttacaacggcaagctgattgcttacccgatcgctgttgaagcgt
tatcgctgatttataacaaagatctgctgccgaacccgccaaaaacctgggaagagatcccggcgctg
gataaagaactgaaagcgaaaggtaagagcgcgctgatgttcaacctgcaagaaccgtacttcacctg
gccgctgattgctgctgacgggggttatgcgttcaagtatgaaaacggcaagtacgacattaaagacg
tgggcgtggataacgctggcgcgaaagcgggtctgaccttcctggttgacctgattaaaaacaaacac
atgaatgcagacaccgattactccatcgcagaagctgcctttaataaaggcgaaacagcgatgaccat
caacggcccgtgggcatggtccaacatcgacaccagcaaagtgaattatggtgtaacggtactgccga
ccttcaagggtcaaccatccaaaccgttcgttggcgtgctgagcgcaggtattaacgccgccagtccg
aacaaagagctggcgaaagagttcctcgaaaactatctgctgactgatgaaggtctggaagcggttaa
taaagacaaaccgctgggtgccgtagcgctgaagtcttacgaggaagagttggcgaaagatccacgta
ttgccgccaccatggaaaacgcccagaaaggtgaaatcatgccgaacatcccgcagatgtccgctttc
tggtatgccgtgcgtactgcggtgatcaacgccgccagcggtcgtcagactgtcgatgaagccctgaa
agacgcgcagactggtggttctggtgcaagcattgctcagaacggtcatgtgaatggggacgttgtag
ccatcgatttttgtaaaaagtccatccacgatccactgaactgggaaatggctgcggagtcattgaaa
ggctcgcaccttgacgaagttaaaaagatggtcgacgaatttcgtaaacctatcgttaaactcggtgg
cgagactctgacggtagcacaagtggcttctattgcgaacgcagataataaaacctgcggtgctaaag
tcgaattatctgaacgcgctcgtgcaggcgtgaaggcgagcagtgactgggttatggattctatgtgc
aaagggacagactcctacggtgtaactaccggcttcggtgccacttctcaccgccgtaccaaaaacgg
tggcgcgctgcaaaaagaactgatacggttcctgaacgctggcgttttcggcaacggtaccgagagct
gccatactctgccgcactccgctacccgtgcggcgatgctagtgagaatcaatactctcctgcaagga
tattcgggtatccgtttcgaaattctggaagctattacaaagctgatcaacagcaacatcactccgtg
tctgccgctgaggggcaccatcacggcctccggagacttggttcccctttcttacatcgctggtctgc
tgacagggcgcccgaacagcaaagccgtgggccctaacggcgaaaagctaaatgcagaggaagcgttc
cgagttgctggtgtatctggtggttttttcgaacttcagccgaaagagggcttagcactggttaacgg
caccgctgtcggttccggtatggcatcaatggtactgttcgaatcaaacattctggctgtaatgagtg
aggtgttgtctgccatctttgctgaagttatgaacggcaaaccggaatttactgatcacctcacgcat
aagctgaaacaccacccaggccagatcgaggcggctgcgattatggaacatatactggatggctccag
ctatgtcaaagcagcacagaaactgcacgaaatggaccctctgcaaaaaccgaaacaggaccgctacg
cgctgcgtaccagcccgcagtggctggggccgcagatcgaagtcatccgtgccgctactaaaatgatt
gaacgcgaaatcaacagcgttaatgataacccgctgatcgacgtatcgcgtaacaaagcactgcatgg
tggtaatttccagggcactcctattggggtgtccatggacaacacccgtctggccctggcatcaattg
gtaaactgatgtttgcccagttcagcgaacttgttaacgattactataataacggtctgccgtctaac
ctgacggcgggtcgcaacccatctctcgactacggcttcaagggtgctgagatcgcaatggctagtta
ctgctctgagctgcaattccttgcgaacccagtcaccaatcacgttcagtcagccgagcagcacaacc
aggatgttaactccctgggcttgatctctgcacgcaaaacggccgaagctgtagatatcctgaaacta
atgagttctacttatctggttgctttatgccaggctattgacctgcgtcatcttgaagaaaatctgaa
gaacgcggtaaagaacacagtgagccaggttgcaaaacgtactctaaccatgggtgccaacggcgagc
tgcacccggctcgtttctgtgaaaaagaactgctgcgtgttgtggatcgagagtacttattcgcctac
gcggatgacccgtgcagcagcacctatccgctgatgcagaagctgcgtcaggtactggtggatcatgc
actgaacaacggcgaaagcgaaaagaatgttaacagctctatctttcagaaaatcgctgctttcgaag
atgagctcaaagcggtgctgcccaaagaagtggaatctgctcgcattacactggaatctgggaatcca
tctataccgaaccgtatcactgagtgtcgcagctacccgctttatcgtttggtgcgcaaagagctggg
cactgaattgctgaccggcgaacgtgttcgctcccccggtgaagaaattgataaagtattcaccgcca
tgtgcaacggtcaaatcatcgacccgctgttagaatgcctgaaatcctggaacggcgctccgctgccg
atctgc

In some embodiments, the PAL enzyme is a Salvia miltiorrhiza (Chinese sage) PAL or SmPAL. The Salvia miltiorrhiza PAL is provided by SEQ ID NO: 11, which corresponds to the sequence provided by UniProtKB Accession No. A9XIW5 (expressed in strain t732611 described in the Examples):

MAAENGHHEESNGFCVKQNDPLNWVAAAESLKGSHLDEVKRMVEEFRKPVVKLGGETLTISQVAAIAA
KDNAVAVELVESSRAGVKASSDWVMESMSKGTDSYGVTTGFGATSHRRTKQGGALQKELIRFLNAGIF
GNGTESNHTLPHTATRAAMLVRINTLLQGYSGIRFEILEAITKFLNENITPCLPLRGTITASGDLVPL
SYIAGLLTGRPNSKAVGPNGEPLNAEEAFKLAGVKGGFFELQPKEGLALVNGTAVGSGLASIALFDAN
ILAVLSEVMSAVFAEVMNGKPEFTDHLTHKLKHHPGQIEAAAIMEHILDGSGYVKAAQKLHEQDPLQK
PKQDRYALRTSPQWLGPQIEVIRTATKMIEREINSVNDNPLIDVSRNKALHGGNFQGTPIGVSMDNAR
LAIASIGKLLFAQFSELVNDLYNNGLPSNLSGGRNPSLDYGFKGSEIAMASYCSELQFLANPVTNHVQ
SAEQHNQDVNSLGLISSRKTVEALDILKLMSSTYLVALCQAVDLRHLEENLKHAVKNTVSQVAKRTLT
MGVNGELHPSRFCEKDLIRVVDREYVFAYIDDPSSATYPLMQKLRQVLVDHALKNGDLEKNASTSIFQ
KIEAFEEELKALLPKEVGSARMALESGSPTVANRIAECRSYPLYKFIREQLGAGFLTGEKAVSPGEEC
EKVFTALSNGLIIDPLLECLQGWNGQPLPIC

A non-limiting example of a nucleotide sequence encoding SEQ ID NO: 11 is provided by SEQ ID NO: 12:

atggcggccgagaatggccaccatgaagaaagcaacggtttctgcgttaaacaaaacgacccactcaa
ctgggtcgctgctgcggaatcgttgaaggggtcacacttagacgaagtaaaaaggatggtggaggaat
ttcgcaaacctgtggttaaacttggcggtgaaacgctgacaatcagtcaggtagcagctattgcagct
aaggataacgcagttgctgttgagctggtggaatcctctcgggcgggtgttaaagccagctccgattg
ggtgatggagtctatgagcaaaggcactgactcttatggagttactaccggcttcggtgccacctcac
accgtcgtactaaacagggtggcgcgctgcaaaaggaactgatccgctttctgaatgctgggatattc
ggtaacggcaccgaatccaaccataccctgccgcacacggctactcgagcagctatgctcgtacgtat
caataccctgctgcaaggttacagcgggattcgtttcgaaatcctggaggccatcactaaatttttga
acgaaaacattaccccctgtctgccactgcgcggcaccatcactgcatctggcgatctggtcccgcta
tcttacattgcaggcctgttgactggccgtccgaactccaaagcggtaggtccgaacggtgaaccgct
gaacgccgaagaggctttcaagctggctggtgttaaaggtggatttttcgaattacagcctaaagaag
gcctggctctggtcaatggtacggcggttgggtctggactggcctcgattgctctgttcgatgcgaac
atcctggctgtgctgagcgaagtgatgagtgccgttttcgcggaagtgatgaacggcaaaccggaatt
tacagaccatcttactcacaaactgaaacatcacccgggtcagatcgaggcggcagcaatcatggaac
atattctcgatggtagcggctatgtaaaagctgcgcagaagctacacgaacaggacccgctgcaaaaa
ccaaaacaggaccgctacgccctgcgtacttccccgcagtggctgggtccccagatcgaggttatccg
taccgcaaccaagatgatcgagcgagagattaattctgtaaacgataacccgctgatcgatgtttccc
gtaataaagcactgcacggcggcaactttcagggtactccgattggcgtgtctatggacaacgctcgc
cttgcgatcgctagcatcggtaagctgttgttcgctcagttcagcgaactggtaaacgacttgtacaa
caatggactgccgtctaacttatcaggtggacgcaacccgtccctggattatgggtttaagggctccg
aaatcgcgatggctagctactgctccgaactgcaattcttagccaaccctgtcaccaatcacgtccag
agcgctgagcagcacaaccaggacgttaactcgctgggcctgattagttcccgtaaaacggtggaagc
tctcgatatcctgaaactgatgtcgtctacgtatcttgttgccctgtgccaggcagtagatctgcgcc
atctggaagaaaacctgaaacacgcagtgaaaaacaccgtcagtcaagttgccaaacgtaccctgaca
atgggggttaacggcgaactgcatccttctcgtttctgtgaaaaagacttgataagagtagttgaccg
cgaatacgtctttgcttacatcgatgacccatcatctgcaacttatccgctgatgcagaagctgcgtc
aggttctggttgatcatgcactaaaaaacggtgacctcgagaagaacgcgagcacctcaattttccaa
aaaatcgaggccttcgaagaagaactgaaagctttactgcctaaagaagtaggctctgcaagaatggc
gcttgagagcggttccccgactgtggctaaccgtattgcggagtgccgtagttacccgctatataagt
tcattcgtgaacagctgggtgcgggcttcctgacaggtgaaaaagctgtttctccgggggaagaatgc
gaaaaggtttttacggctctctccaatggcctcatcatcgatccgcttctggaatgtctgcaaggttg
gaacggccagccattgcctatatgc

In some embodiments, the PAL enzyme is a Ricinus communis (Castor bean) PAL or RcPAL. The Ricinus communis PAL is provided by SEQ ID NO: 13, which corresponds to the sequence provided by UniProtKB Accession No. B9S0K2 (expressed in strain t726556 described in the Examples):

MAAMAENGSKNDSLESFCNMGRDPLSWGLAAESMKGSHLDEVKKMVAEYRKPFVKLGGETLIVAQVAA
IASHDCGVKVELSESARAGVKASSDWVMDSMNKGTDSYGVTTGFGATSHRRTKQGGALQKELIRFLNA
GIFGNGTESCHTLPHSATRAAMLVRINTLLQGYSGIRFEILEAITKLLNHNITPCLPLRGTITASGDL
VPLSYIAGLLTGRPNSKAIGPNGESMDALEAFRLAGIESGFFELQPKEGLALVNGTAVGSGLASMVLF
EANILAVLSEILSAIFAEVMNGKPEFTDHLTHKLKHHPGQIEAAAIMEHILDGSSYVKAAKKLHEMDP
LQKPKQDRYALRTSPQWLGPQIEVIRFSTKSIEREINSVNDNPLIDVSRNKALHGGNFQGTPIGVSMD
NARLAIASIGKLMFAQFSELVNDFYNNGLPSNLTAGRNPSLDYGFKGAEIAMASYCSELQYLANPVTS
HVQSAEQHNQDVNSLGLISSRKTAEAVDILKLMSTTYLVALCQAIDLRHLEENLRQAVKNTVSQVAKR
VLTTGANGELHPSRFCEKDLLKVVDREYVFAYADDPCSATYPLMQKLRQILVEHALANGENEKNAGTS
VFQKISAFEEELKILLPKEVESVRIAYESGNPATANRIKECRSYPLYKFVREELGTGLLTGDKVMSPG
EEFDKVFTAMCQGKIIDPMMDCLKEWNGAPLPIC

A non-limiting example of a nucleotide sequence encoding SEQ ID NO: 13 is provided by SEQ ID NO: 14:

atggcagctatggccgagaacggtagtaaaaatgattcactggaatcgttctgtaacatgggccgcga
cccattatcttggggtttggcggctgaatctatgaagggcagccatctggacgaagtaaaaaagatgg
tggcggagtatcggaaaccttttgttaaactgggcggggaaacactgacggttgctcaggtcgcagct
atcgcgtcccacgattgcggtgttaaagtagaactgagcgaatctgcccgtgcaggagttaaagctag
ctccgactgggtgatggatagcatgaacaaaggcaccgacagctacggtgtcactactggtttcggcg
caacctcccacaggcgcaccaagcaaggaggtgctctccagaaagagctaattcgttttctgaatgct
ggcatcttcggtaacggcactgaatcttgccacaccctgccccatagtgcgactcgtgcagcgatgct
ggtgcgcattaacacgctgcttcagggttacagcggcatccgtttcgagatcctcgaagctattacca
aactgctgaaccacaatatcactccgtgccttccgttacgtggtacaatcaccgcctctggagatttg
gttccgctgagctacattgccggcctgttaaccggtcgtccgaactccaaagcgatcggtccgaacgg
ggaatctatggatgcgctggaagcttttcgactagccggcatagaatcaggtttcttcgaactgcaac
cgaaggaaggcctggcactggtgaacggcactgctgttgggagtggtctggcctccatggtactgttc
gaggcgaatatcctggcagttttatctgaaatcctgtcggccatttttgcagaagtaatgaacggcaa
accggaatttacggaccatctgactcacaagctgaaacaccatccaggtcagattgaggctgctgcaa
tcatggaacacatacttgatggctcctcttatgtcaaagcggctaaaaaattacatgagatggaccct
ctgcaaaagccgaaacaggaccgttacgctttgcgcaccagccctcagtggctggggccacagatcga
agtaatccgtttctcaactaaatccatcgaacgcgagattaactctgtcaacgataacccgctgatcg
atgttagccgaaacaaggcactgcacggtggcaactttcagggcacccctattggcgttagtatggat
aatgctcgtctggcaatcgcgtctatcggtaaattaatgttcgcgcagttctccgagctcgtaaatga
cttttacaacaacggtctgccgtcaaacctgaccgctggccgtaatccgtcgctcgactatggtttca
agggcgctgaaatcgctatggcgagttactgctccgaattgcaatatctggcgaacccggtgaccagc
cacgttcagtctgctgaacagcacaaccaggacgttaactcactgggtctgatcagcagccgcaaaac
agccgaggcagttgacattctgaaactgatgtctactacctacctggtcgctctttgtcaggcaattg
atctacgtcatctggaggaaaatctgcgccaggctgtgaaaaacactgtgtctcaagttgccaagcgt
gtcttaaccacaggagcgaacggcgagctgcatccaagccgtttctgcgaaaaagatctgctcaaagt
ggtggaccgcgaatatgtattcgcatacgccgatgatccgtgttccgcgacgtatcccctgatgcaga
aactgaggcaaattctggtagaacacgctctggcgaacggtgaaaacgagaagaacgctggcacctcg
gttttccagaaaatctctgcttttgaagaagaactgaaaactctactgccaaaagaagttgaatccgt
ccgtatcgcctacgaaagcggtaacccggcaactgctaaccgtattaaagaatgccgctcgtacccgt
tgtacaaattcgtgcgagaggaactgggcactggacttctgactggtgataaggtgatgtcccctggg
gaggagtttgacaaggtattcacggctatgtgccaggggaaaatcattgacccgatgatggactgcct
gaaagaatggaacggtgctccgctgccgatatgt

In some embodiments, the PAL enzyme is a Vitis vinifera (Grape) PAL or VvPAL. The Vitis vinifera PAL is provided by SEQ ID NO: 15 (expressed in strain t732247 described in the Examples):

MNCHGSKKVESFVVSDPLNWGVAAEALKGSHLDEVKRMVAEYRKPVVRLGGETLTISQVAAIAGREAD
VSVELSETARAGVNASSEWVMESMSKGTDSYGVTTGFGATSHRRTKQGGALQKELIRFLNAGIFGNGT
ESCHTLPHSATRAAMLVRINTLLQGYSGIRFEILEAITKLLNHNITPCLPLRGTVTASGDLVPLSYIA
GLLTGRPNSKAVGPSGEVVNAEEAFKMAGIESGFFELQPKEGLALVNGTAVGSGLASMVLFETNVLAV
LSEVLSAIFAEVMQGKPEFTDHLTHKLKHHPGQIEAAAIMEHILDGSSYVKEAKKLHEMDPLQKPKQD
RYALRISPQWLGPQIEVIRASTKSIEREINSVNDNPLIDVSRNKALHGGNFQGTPIGVSMDNTRLAIA
AIGKLMFAQFSELVNDFYNNGLPSNLSGSRNPSLDYGFKGAEIAMASYCSELQFLANPVTNHVQSAEQ
HNQDVNSLGLISSRKTAEAVDILKLMSTTYLVALCQAIDLRHLEENLKSTVKKTVSHVAKKTLTTGAN
GELHPSRFCEKALLKVVDREHVFAYIDDPCSATYPLMQKVRQVLVEHALNNGENEKNGSTSIFQKIVA
FEEELKAVLPKEVESARGGVESGNPSIPNRIRECRSYPLYKFVREELGTGLLTGEKVRSPGEDFDKVF
TAMCEGKIIDPLLDCLSAWNGAPLPIC

A non-limiting example of a nucleotide sequence encoding SEQ ID NO: 15 is provided by SEQ ID NO: 16:

atgaactgccatggttccaagaaagtagagagctttgtcgttagtgacccactgaattggggagtggc
tgccgaagcgttaaaaggctctcacctcgatgaagttaaacgtatggtagcagaatatcgcaagcccg
tggttcgtctagggggtgaaacgttgaccatctctcaggtcgctgcgattgccggtcgcgaggctgac
gttagcgtagaactgtcagagacagcacgagctggcgtcaacgcgtcgtccgaatgggttatggaaag
catgtctaaaggcactgatagttacggtgtgactaccggcttcggtgcaacttctcaccgtcgtacca
aacaaggcggtgctcttcagaaagaactgatacgcttcctgaacgcgggcatctttggtaacggtacc
gagagctgtcatactttgccgcactccgcaacgcgtgccgctatgctggttagaattaatactttact
gcaaggctactccggaatcaggttcgaaatcctggaggctatcaccaaactgctgaaccacaacatca
caccttgccttccgctgcgcggcaccgtaaccgcgtccggtgacctcgtgccgctgagctatattgca
ggcctgctgactgggcgtccgaatagcaaagctgttggtccatcgggtgaagtggttaacgcagaaga
ggcgttcaagatggccggaatcgaatctggtttctttgaactgcaaccgaaggaaggcctggctcttg
taaacggcacggcagttggctctggtttagctagcatggtgctctttgaaactaacgtactggcagta
ctgtctgaagtgctgtccgccattttcgctgaagttatgcagggtaaaccggaatttaccgatcacct
gacccataaactgaaacaccacccgggccagattgaggctgctgcgatcatggagcatatcctggacg
gctcaagttacgtcaaagaagcgaaaaaactgcacgaaatggatcctctgcaaaagccgaaacaggac
cggtacgctctgcgtacttccccacagtggctgggtccgcagatagaggtgatccgtgcttctactaa
aagtattgagcgcgaaatcaattcggtaaacgataacccgttaatcgacgttagccgcaacaaagctc
tgcatggaggtaacttccaggggacaccgatcggcgtcagcatggacaatacccgtctggccattgcc
gcaatcggcaagctgatgttcgcccagttttctgaactggtaaacgatttctacaacaatggtctgcc
atctaacctgtccggtagcagaaacccctctctcgattatgggttcaaaggcgccgaaatcgcaatgg
cttcgtattgctcagaattgcagtttctggcgaacccggttaccaatcacgttcagtcggctgagcag
cacaaccaagatgtgaactcattgggtctgattagcagccgtaaaaccgcggaggcggttgatattct
gaaactgatgtctactacgtacctcgtggctctgtgccaggcgatcgacctacgtcatctcgaagaaa
accttaagtccaccgttaaaaagactgtcagccatgttgctaagaaaaccctgacaacgggcgcaaat
ggcgaactgcacccgtctaggttctgtgagaaagccctgcttaaggtcgttgaccgcgaacacgtatt
tgcatatatcgacgatccttgctctgctacttacccactgatgcagaaagtacgtcaggtgctggtcg
agcacgctctgaacaacggcgaaaacgaaaagaacggttcaacctcgatcttccagaaaatagttgca
ttcgaagaagagctgaaagcggtgttacctaaagaagtcgaatctgcacgtggtggagttgaaagcgg
gaacccgtctattcccaaccgcatccgtgaatgtcgttcctacccgctatacaagttcgtgcgtgaag
aactgggcactggtcttctaaccggtgaaaaagtacgcagtcctggcgaagacttcgacaaagttttt
actgccatgtgcgaggggaaaatcatcgacccgctgctggattgtctatcggcctggaacggcgcgcc
qctqccgatctqc

In some embodiments, the PAL enzyme is a Photorhabdus luminescens subsp. laumondii PAL or P1PAL. The Photorhabdus luminescens subsp. laumondii PAL is provided by SEQ ID NO: 17, which corresponds to the sequence provided by UniProtKB Accession No. Q7N4T3 (expressed in strain t720968 described in the Examples):

MKAKDVQPTIIINKNGLISLEDIYDIAIKQKKVEISTEITELLTHGREKLEEKLNSGEVIYGINTGFG
GNANLVVPFEKIAEHQQNLLTFLSAGTGDYMSKPCIKASQFTMLLSVCKGWSAIRPIVAQAIVDHINH
DIVPLVPRYGSVGASGDLIPLSYIARALCGIGKVYYMGAEIDAAEAIKRAGLTPLSLKAKEGLALING
TRVMSGISAITVIKLEKLFKASISAIALAVEALLASHEHYDARIQQVKNHPGQNAVASALRNLLAGST
QVNLLSGVKEQANKACRHQEITQLNDTLQEVYSIRCAPQVLGIVPESLATARKILEREVISANDNPLI
DPENGDVLHGGNFMGQYVARTMDALKLDIALIANHLHAIVALMMDNRFSRGLPNSLSPTPGMYQGFKG
VQLSQTALVAAIRHDCAASGIHTLATEQYNQDIVSLGLHAAQDVLEMEQKLRNIVSMTILVVCQAIHL
RGNISEIAPETAKFYHAVREISSPLITDRALDEDIIRIADAIINDQLPLPEIMLEE

A non-limiting example of a nucleotide sequence encoding SEQ ID NO: 17 is provided by SEQ ID NO: 18:

atgaaggccaaagatgtacagccaacgatcattataaataaaaacggcctgatcagcttggaggacat
ctatgacattgcaattaaacaaaagaaagtcgaaatctcgacagaaatcactgaactgctgactcatg
gtcgtgaaaaactagaggaaaaactgaactccggcgaagttatctacggtattaacaccgggttcggt
ggaaacgctaatctggttgtgccttttgaaaaaatcgcggaacaccagcagaacttactgaccttcct
ctcagctggcactggtgattacatgtctaagccgtgtatcaaagcgagccaatttaccatgctgctgt
ctgtatgcaaaggctggtccgccacccgccccattgttgctcaggctatcgtggaccacattaaccac
gatattgtaccgcttgttccgaggtatggctctgtcggtgcaagtggtgacctgatcccgctgtctta
catcgcacgcgctctgtgcggcattggtaaagtgtactatatgggcgccgagatcgatgcggctgaag
caatcaagcgtgcaggcctgactccgttgagcttgaaagcgaaggagggtctggctctgatcaacggg
acacgtgttatgagcggcatttccgcgataaccgtcatcaaactggaaaaattattcaaagcaagtat
ctcagctatcgcgctggctgttgaagcgctgctggccagccatgagcattacgacgcgcgtatccagc
aggtcaaaaaccacccaggtcagaatgctgttgcctccgcactgagaaaccttctggctggatctact
caggttaacctcctgagtggtgtaaaagaacaggcgaataaagcatgtcgccaccaggaaattaccca
gctaaacgatacgctacaagaagtgtactctatacgatgcgctccgcaagtgctcgggatcgtacccg
aatccctggctactgcgcgtaagattcttgagcgtgaggttatctcagctaacgacaaccctttaatc
gatccggaaaacggagatgtcctgcacggtggcaacttcatgggccagtatgtagcacgtaccatgga
cgccctgaaactggatattgctctgattgcaaaccatctgcatgctatcgttgcgctgatgatggaca
accgcttcagccgcggtctgccgaatagcctgtctccgaccccgggcatgtaccaaggttttaagggc
gtacagctgtcgcagactgcgcttgtagctgctatccgtcacgactgcgccgcaagcggtatccacac
cctcgcaaccgaacagtacaatcaggatatcgtgagcttgggcctgcacgcagctcaggacgttctgg
aaatggaacagaagctgcgtaacatcgtgtccatgactatcttagttgtctgccaggccattcacctg
agaggcaatatttctgaaattgcgccagaaacggcaaaattctaccacgctgttcgtgagatctcgtc
ccctctgatcacagatcgtgctctcgacgaggatatcattcgcatcgcagacgcgatcattaacgatc
agttaccgctgcccgaaatcatgctggaagaa

It should be appreciated that sequences disclosed in this application may or may not contain secretion signals. The sequences disclosed in this application encompass versions with or without secretion signals. It should also be understood that amino acid sequences disclosed in this application may be depicted with or without a start codon (M). The sequences disclosed in this application encompass versions with or without start codons. Accordingly, in some instances amino acid numbering may correspond to amino acid sequences containing secretion signal and/or a start codon, while in other instances, amino acid numbering may correspond to amino acid sequences that do not contain a secretion signal and/or a start codon. It should also be understood that sequences disclosed in this application may be depicted with or without a stop codon. The sequences disclosed in this application encompass versions with or without stop codons.

In some embodiments, a host cell that expresses a heterologous polynucleotide encoding a PAL enzyme may increase conversion of L-phenylalanine to trans-cinnamic acid by 1.1-fold, 1.2-fold, 1.3-fold, 1.4-fold 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, the control is a host cell that expresses a heterologous polynucleotide encoding SEQ ID NO: 3 or 17. In some embodiments, the control is an E. coli Nissle strain SYN107 which is described in and incorporated by reference from US Patent Publication No. 2017/0312320.

In some embodiments, a host cell that expresses a heterologous polynucleotide encoding a PAL enzyme may exhibit at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on L-phenylalanine relative to other amino acids. In some embodiments, a host cell that expresses a heterologous polynucleotide encoding a PAL enzyme may exhibit at least 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity on L-phenylalanine relative to other amino acids.

In some embodiments, a PAL comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to any one of SEQ ID NO: 1, 3, 5, 7, 9, 11, 13, 15, or 17, any one of SEQ ID NO: 2, 4, 6, 8, 10, 12, 14, 16, or 18, an amino acid or polynucleotide sequence of a PAL enzyme in Table 3 or Table 5, or a PAL enzyme otherwise described in this disclosure.

In some embodiments, a PAL enzyme comprises at least 1, at least 2, at least 3, at least 4, at least 5, at least 6, at least 7, at least 8, at least 9, at least 10, at least 11, at least 12, at least 13, at least, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, insertions, or additions relative to any one of SEQ ID NO: 1, 3, 5, 7, 9, 11, 13, 15, or 17, a PAL enzyme in Table 3 or Table 5, or a PAL enzyme otherwise described in this disclosure.

In some embodiments, a PAL enzyme comprises: P at a residue corresponding to residue 4 in SEQ ID NO: 3; V at a residue corresponding to residue 77 in SEQ ID NO: 3; Y at a residue corresponding to residue 84 in SEQ ID NO: 3; L at a residue corresponding to residue 87 in SEQ ID NO: 3; S at a residue corresponding to residue 88 in SEQ ID NO: 3; T at a residue corresponding to residue 90 in SEQ ID NO: 3; V at a residue corresponding to residue 91 in SEQ ID NO: 3; E at a residue corresponding to residue 96 in SEQ ID NO: 3; A at a residue corresponding to residue 98 in SEQ ID NO: 3; E at a residue corresponding to residue 102 in SEQ ID NO: 3; K at a residue corresponding to residue 102 in SEQ ID NO: 3; R at a residue corresponding to residue 102 in SEQ ID NO: 3; S at a residue corresponding to residue 102 in SEQ ID NO: 3; A at a residue corresponding to residue 104 in SEQ ID NO: 3; M at a residue corresponding to residue 104 in SEQ ID NO: 3; F at a residue corresponding to residue 105 in SEQ ID NO: 3; Q at a residue corresponding to residue 106 in SEQ ID NO: 3; A at a residue corresponding to residue 108 in SEQ ID NO: 3; H at a residue corresponding to residue 108 in SEQ ID NO: 3; Q at a residue corresponding to residue 108 in SEQ ID NO: 3; T at a residue corresponding to residue 108 in SEQ ID NO: 3; V at a residue corresponding to residue 108 in SEQ ID NO: 3; S at a residue corresponding to residue 110 in SEQ ID NO: 3; H at a residue corresponding to residue 158 in SEQ ID NO: 3; V at a residue corresponding to residue 158 in SEQ ID NO: 3; V at a residue corresponding to residue 165 in SEQ ID NO: 3; A at a residue corresponding to residue 172 in SEQ ID NO: 3; G at a residue corresponding to residue 175 in SEQ ID NO: 3; T at a residue corresponding to residue 175 in SEQ ID NO: 3; Q at a residue corresponding to residue 214 in SEQ ID NO: 3; A at a residue corresponding to residue 218 in SEQ ID NO: 3; S at a residue corresponding to residue 218 in SEQ ID NO: 3; I at a residue corresponding to residue 219 in SEQ ID NO: 3; I at a residue corresponding to residue 222 in SEQ ID NO: 3; L at a residue corresponding to residue 222 in SEQ ID NO: 3; N at a residue corresponding to residue 222 in SEQ ID NO: 3; T at a residue corresponding to residue 222 in SEQ ID NO: 3; V at a residue corresponding to residue 222 in SEQ ID NO: 3; L at a residue corresponding to residue 243 in SEQ ID NO: 3; A at a residue corresponding to residue 253 in SEQ ID NO: 3; S at a residue corresponding to residue 345 in SEQ ID NO: 3; H at a residue corresponding to residue 364 in SEQ ID NO: 3; M at a residue corresponding to residue 394 in SEQ ID NO: 3; V at a residue corresponding to residue 406 in SEQ ID NO: 3; A at a residue corresponding to residue 407 in SEQ ID NO: 3; C at a residue corresponding to residue 407 in SEQ ID NO: 3; S at a residue corresponding to residue 407 in SEQ ID NO: 3; T at a residue corresponding to residue 407 in SEQ ID NO: 3; G at a residue corresponding to residue 413 in SEQ ID NO: 3; S at a residue corresponding to residue 413 in SEQ ID NO: 3; H at a residue corresponding to residue 415 in SEQ ID NO: 3; H at a residue corresponding to residue 422 in SEQ ID NO: 3; L at a residue corresponding to residue 423 in SEQ ID NO: 3; T at a residue corresponding to residue 424 in SEQ ID NO: 3; A at a residue corresponding to residue 450 in SEQ ID NO: 3; G at a residue corresponding to residue 450 in SEQ ID NO: 3; H at a residue corresponding to residue 450 in SEQ ID NO: 3; A at a residue corresponding to residue 453 in SEQ ID NO: 3; S at a residue corresponding to residue 453 in SEQ ID NO: 3; P at a residue corresponding to residue 522 in SEQ ID NO: 3; and/or L at a residue corresponding to residue 529 in SEQ ID NO: 3.

In some embodiments, a PAL enzyme comprises: I at a residue corresponding to residue 40 in SEQ ID NO: 17; G at a residue corresponding to residue 92 in SEQ ID NO: 17; V at a residue corresponding to residue 111 in SEQ ID NO: 17; F at a residue corresponding to residue 133 in SEQ ID NO: 17; M at a residue corresponding to residue 133 in SEQ ID NO: 17; K at a residue corresponding to residue 167 in SEQ ID NO: 17; I at a residue corresponding to residue 258 in SEQ ID NO: 17; R at a residue corresponding to residue 258 in SEQ ID NO: 17; T at a residue corresponding to residue 263 in SEQ ID NO: 17; S at a residue corresponding to residue 275 in SEQ ID NO: 17; S at a residue corresponding to residue 288 in SEQ ID NO: 17; L at a residue corresponding to residue 402 in SEQ ID NO: 17; I at a residue corresponding to residue 432 in SEQ ID NO: 17; S at a residue corresponding to residue 433 in SEQ ID NO: 17; A at a residue corresponding to residue 470 in SEQ ID NO: 17; P at a residue corresponding to residue 499 in SEQ ID NO: 17; and/or V at a residue corresponding to residue 502 in SEQ ID NO: 17.

Variants Variants of enzymes described in this disclosure (e.g., PAL enzymes, including variants to nucleic acid and amino acid sequences) are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence (e.g., PAL sequence). In some embodiments, sequence identity is determined over a region (e.g., a stretch of amino acids or nucleic acids, e.g., the sequence spanning an active site) of a sequence (e.g., PAL sequence).

Identity can also refer to the degree of sequence relatedness between two sequences as determined by the number of matches between strings of two or more residues (e.g., nucleic acid or amino acid residues). Identity measures the percent of identical matches between two or more sequences with gap alignments (if any) addressed by a particular mathematical model or computer program (e.g., algorithms).

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST© can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this disclosure, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art, such as, for example, Clustal Omega or BLAST®.

As used in this disclosure, variant sequences may be homologous sequences. As used in this disclosure, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant (e.g., PAL enzyme variant) comprises a domain that shares a secondary structure (e.g., alpha helix, beta sheet) with a reference polypeptide (e.g., a reference PAL enzyme). In some embodiments, a polypeptide variant (e.g., PAL enzyme variant) shares a tertiary structure with a reference polypeptide (e.g., a reference PAL enzyme). As a non-limiting example, a variant polypeptide (e.g., PAL enzyme variant) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Any suitable method, including circular permutation (Yu and Lutz, Trends Biotechnol. 2011 January; 29(1):18-25), may be used to produce such variants. In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two polypeptides, however, may reveal that their tertiary structure is similar. Without being bound by a particular theory, a variant polypeptide created through circular permutation of a reference polypeptide and with a tertiary structure similar to the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling. Variants described in this application include circularly permutated variants of sequences described in this application.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant PAL enzyme disclosed in this application are also encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g., PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, a PAL enzyme coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference (e.g., PAL enzyme) coding sequence. In some embodiments, the PAL enzyme coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference (e.g., PAL enzyme) coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., PAL enzyme) relative to the amino acid sequence of a reference polypeptide (e.g., PAL enzyme).

In some embodiments, the one or more mutations in a recombinant PAL enzyme sequence alters the amino acid sequence of the polypeptide (e.g., PAL enzyme) relative to the amino acid sequence of a reference polypeptide (e.g., PAL enzyme). In some embodiments, the one or more mutations alters the amino acid sequence of the recombinant polypeptide (e.g., PAL enzyme) relative to the amino acid sequence of a reference polypeptide (e.g., PAL enzyme) and alters (enhances or reduces) an activity of the polypeptide relative to the reference polypeptide.

The activity (e.g., specific activity) of any of the recombinant polypeptides described in this disclosure (e.g., PAL enzyme) may be measured using routine methods. As a non-limiting example, a recombinant polypeptide's activity may be determined by measuring its substrate specificity, product(s) produced, the concentration of product(s) produced, or any combination thereof. As used in this disclosure, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide (e.g., PAL enzyme) coding sequence may result in conservative amino acid substitutions to provide functionally equivalent variants of the foregoing polypeptides, e.g., variants that retain the activities of the polypeptides. As used in this disclosure, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group include lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

Non-limiting examples of functionally equivalent variants of polypeptides may include conservative amino acid substitutions in the amino acid sequences of proteins disclosed in this application. As used in this disclosure “conservative substitution” is used interchangeably with “conservative amino acid substitution” and refers to any one of the amino acid substitutions provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Conservative Amino Acid Substitutions.

| Original | | Conservative Amino |
|---|---|---|
| Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R group | Ala, Ser |
| Gln (Q) | polar uncharged R group | Asn, Asp, Glu |
| Glu (E) | negatively charged R group | Asn, Asp, Gln |
| Gly (G) | nonpolar aliphatic R group | Ala, Ser |
| His (H) | positively charged R group | Arg, Tyr, Trp |
| Ile (I) | nonpolar aliphatic R group | Leu, Met, Val |
| Leu (L) | nonpolar aliphatic R group | Ile, Met, Val |
| Lys (K) | positively charged R group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

Amino acid substitutions in the amino acid sequence of a polypeptide to produce a recombinant polypeptide (e.g., PAL enzyme) variant having a desired property and/or activity can be made by alteration of the coding sequence of the polypeptide (e.g., PAL enzyme). Similarly, conservative amino acid substitutions in the amino acid sequence of a polypeptide to produce functionally equivalent variants of the polypeptide typically are made by alteration of the coding sequence of the recombinant polypeptide (e.g., PAL enzyme).

Mutations (e.g., substitutions, additions, and/or deletions) can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing techniques, or by insertions, such as insertion of a tag (e.g., a HIS tag or a GFP tag).

### Polynucleotides Encoding PAL Enzymes

Aspects of the present disclosure relate to recombinant enzymes, functional modifications and variants thereof, as well as uses relating thereto. For example, the enzymes and cells described in this application may be used to promote L-phenylalanine consumption, e.g., by converting L-phenylalanine to trans-cinnamic acid. The methods may comprise using a host cell comprising one or more enzymes disclosed in this application, a cell lysate, isolated enzymes, or any combination thereof. Methods comprising recombinant expression of polynucleotides encoding an enzyme disclosed in this application in a host cell are encompassed by the present disclosure. Methods comprising administering a host cell comprising at least one PAL enzyme (e.g., a PAL enzyme) to a subject in need thereof are encompassed by the present disclosure. In vitro methods comprising reacting one or more PALs in a reaction mixture disclosed in this application are also encompassed by the present disclosure.

A polynucleotide encoding any one or more of the recombinant polypeptides (e.g., PAL) is encompassed by the disclosure and may be comprised within a host cell. In some embodiments, the polynucleotide is in the form of an operon. In some embodiments, at least one ribosome binding site is present between one or more of the coding sequences present in the polynucleotide.

A polynucleotide encoding any one or more of the recombinant polypeptides (e.g., PAL) described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

A vector encoding any of the recombinant polypeptides (e.g., PAL) described in this application may be introduced into a suitable host cell using any method known in the art. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell (e.g., microbe). In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, a host cell has already been transformed with one or more vectors. In some embodiments, a host cell that has been transformed with one or more vectors is subsequently transformed with one or more vectors. In some embodiments, a host cell is transformed simultaneously with more than one vector. In some embodiments, a cell that has been transformed with a vector or an expression cassette incorporates all or part of the vector or expression cassette into its genome. In some embodiments, the nucleic acid sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

In some embodiments, the polynucleotide encoding any one or more of the proteins described in this application is under the control of regulatory sequences (e.g., enhancer sequences). In some embodiments, a polynucleotide is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Plslcon, T3, T7, SP6, and PL. Non-limiting examples of bacterial promoters include Pbad, PmgrB, Ptrc2, PCI857, Plac/ara, Plac/fnr, Ptac, Ptet, Pcmt, and Pm.

In some embodiments, the promoter is Ptet, which is induced by anhydrotetracycline. In other embodiments, the promoter is Ptet*. The Ptet* promoter is also induced by anhydrotetracycline, but exhibits higher induced strength and lower leaky expression than Ptet. The Ptet and Ptet* promoters are described further in and incorporated by reference from Moon et al. (2012) Nature 491(7423):249-253.

In some embodiments, the promoter is an inducible promoter. As used in this application, an “inducible promoter” is a promoter controlled by the presence or absence of a molecule. This may be used, for example, to controllably induce the expression of an enzyme. In some embodiments, where an inducible promoter is linked to a PAL, the expression of PAL may be induced or not induced at certain times. For example, in some embodiments, expression may not be induced at certain times so that phenylalanine consumption would be limited (e.g., during cell growth). Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters known to one of ordinary skill in the art are also contemplated in this application.

Expression of polynucleotides associated with the disclosure can be enhanced, at least in part, by the presence of an insulator ribozyme. In some embodiments of the disclosure, an insulator ribozyme is inserted downstream of a promoter and upstream of a ribosome binding site (RBS). In some embodiments, the insulator ribozyme increases expression of a polynucleotide associated with the disclosure. In some embodiments, an insulator ribozyme is LtsvJ, SccJ, RiboJ, SarJ, PlmJ, VtmoJ, ChmJ, ScvmJ, SltJ, or PlmvJ, as described in, and incorporated by reference from, Lou et al. (2012) Nat Biotechnol. November; 30(11):1137-1142, doi: 10.1038/nbt.2401 and Clifton et al. (2018) J. Biol. Eng.; 12:23, doi: 10.1186/s13036-018-0115-6. It should be appreciated that other insulator ribozymes known in the art may also be compatible with aspects of the disclosure.

Translation of polynucleotides associated with the disclosure can be enhanced, at least in part, by the presence of an RBS. As used in this disclosure, an “RBS” refers to a regulatory nucleic acid region upstream of a start codon in an mRNA that is involved with recruitment of ribosomes. In some embodiments, an RBS is heterologous. Host cells can express a native RBS, e.g., the RBS in its endogenous context, which provides normal regulation of expression of a gene or operon. Alternatively, an RBS may be an RBS that is different from a native RBS associated with a gene or operon, e.g., the RBS is different from the RBS of a gene or operon in its endogenous context. An RBS can be synthetic. As used in this application, a “synthetic RBS” refers to an RBS that is not known to occur in nature. RBSs are further described in, and incorporated by reference from, Salis et al. (2009) Nat. Biotechnol. 27, 946-950 (2009) and Mutalik et al. (2012) Nat. Methods 10:354. It should be appreciated that other RBSs known in the art may also be compatible with aspects of the disclosure.

Regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed in this application may include 5′ leader or signal sequences. Regulatory sequences may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Host Cells

The disclosed methods and compositions and host cells are exemplified with E. coli cells (e.g., E. coli Nissle 1917), but are, in some embodiments, applicable to other host cells.

Suitable host cells include, but are not limited to: yeast cells, bacterial cells, algal cells, plant cells, fungal cells, insect cells, and animal cells, including mammalian cells. In one illustrative embodiment, suitable host cells include E. coli (e.g., Shuffle™ competent E. coli available from New England BioLabs in Ipswich, Mass or E. coli Nissle 1917 available from German Collection of Microorganisms and Cell Cultures (DSMZ Braunschweig, E. coli DSM 6601)).

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, W138, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

A vector encoding any one or more of the recombinant polypeptides (e.g., PAL) described in this application may be introduced into a suitable host cell using any method known in the art. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

In some embodiments, a bioreactor comprises a cell (e.g., a bacterial cell) or a cell culture (e.g., a bacterial cell culture), such as a cell or cell culture described in this application. In some embodiments, a bioreactor comprises a spore and/or a dormant cell type of an isolated microbe (e.g., a dormant cell in a dry state).

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., bacterial cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product may display some differences from the substrate in terms of solubility, toxicity, cellular accumulation and secretion and in some embodiments can have different fermentation kinetics.

In some embodiments, the cells of the present disclosure are adapted to consume phenylalanine in vivo. In some embodiments, the cells are adapted to produce one or more enzymes for phenylalanine consumption via conversion to trans-cinnamic acid (e.g., PAL). In such embodiments, the enzyme can catalyze reactions for the consumption of phenylalanine by bioconversion in an in vitro or ex vivo process.

Any of the proteins or enzymes of the present disclosure may be expressed in a host cell. As used in this application, a host cell is a cell that can be used to express at least one heterologous polynucleotide (e.g., encoding a protein or enzyme as described in this application). The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species than the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

Any suitable host cell may be used to produce any of the recombinant polypeptides (e.g., PAL) disclosed in this application, including eukaryotic cells or prokaryotic cells.

### Methods

In some aspects, the disclosure provides methods of using host cells. In some embodiments, the disclosure provides a method comprising culturing a host cell described in this application (e.g., a host cell comprising a heterologous polynucleotide encoding a PAL). Methods for culturing cells are described elsewhere in this application. In some embodiments, the disclosure provides a method of producing trans-cinnamic acid from phenylalanine and/or degrading phenylalanine, comprising culturing a host cell described in this application (e.g., a host cell comprising a heterologous polynucleotide encoding a PAL). In some embodiments, the disclosure provides a method of producing p-coumaric acid from tyrosine and/or degrading tyrosine, comprising culturing a host cell described in this application (e.g., a host cell comprising a heterologous polynucleotide encoding a PAL). In some embodiments, the production and culturing occurs in vivo, e.g., in a human subject that has been administered the host cell. In some embodiments, the production occurs ex vivo, e.g., in an in vitro cell culture environment. Compositions, cells, enzymes, and methods described in this application are also applicable to industrial settings, including any application wherein there may be a buildup of amino acids (e.g., phenylalanine).

In some aspects, the disclosure provides a method of treating a metabolic disorder associated with an excess of phenylalanine. In some embodiments, the metabolic disorder is associated with deficiency of an enzymatic activity, e.g., phenylalanine hydroxylase (PAH). In some embodiments, the deficiency of an enzymatic activity is localized to an organ or tissue of a subject, e.g., to the liver or hepatic tissue of the subject. In some embodiments, the metabolic disorder is associated with a deficiency in the synthesis or recycling of tetrahydrobiopterin. In some embodiments, the metabolic disorder is phenylketonuria (PKU), an autosomal recessive genetic disorder typically caused by lack of or decreased PAH activity. In some embodiments, PKU is characterized by symptoms including one or more of tremors, seizures, autism, or chronic psychiatric deformities. Without wishing to be bound by theory, PKU is thought to be caused by neurotoxic levels of L-phenylalanine accumulating due to deficiency in PAH activity, which may be caused by mutations in PAH that decrease or eliminate activity or mutations in related enzymes that produce or recycle the PAH cofactor tetrahydrobiopterin. Providing subjects having PKU or other metabolic disorders associated with an excess of L-phenylalanine with the PAL activity of a PAL enzyme described within this disclosure (e.g., by administering a host cell comprising a PAL enzyme described within this disclosure or a nucleic acid encoding the same) may decrease, eliminate, or prevent one or more (e.g., all) symptoms of PKU or the metabolic disorder. In some embodiments, the metabolic disorder is hyperphenylalaninemia.

In some embodiments, a method of treating a metabolic disorder associated with an excess of phenylalanine comprises delivering the PAL activity of a PAL enzyme described within this disclosure to a subject in need thereof. In some embodiments, the method comprises administering a host cell comprising the PAL enzyme and/or a nucleic acid encoding the PAL enzyme to the subject. In some embodiments, the method comprises administering the PAL enzyme or a nucleic acid encoding the PAL enzyme to the subject. In some embodiments, the method comprises administering a vector (e.g., a viral vector) comprising a nucleic acid encoding the PAL enzyme to the subject. In some embodiments, the method comprises administering a lipid nanoparticle (LNP) comprising the PAL enzyme and/or a nucleic acid encoding the PAL enzyme to the subject. Administration may be accomplished by any mode known in the art and appropriate for the composition being delivered. In some embodiments, administration is parenteral or enteral. In some embodiments, administration is via injection, e.g., intravenous or subcutaneous injection. In some embodiments, a composition described within this disclosure is orally administered.

In some embodiments, a method described within this disclosure comprising delivering PAL activity to a subject further comprises administering one or more alternate PAL substrates to the subject. Without wishing to be bound by theory, some PAL enzymes catalyze reactions that consume tyrosine and/or histidine, and an increase in PAL activity in a subject may decrease the levels of one or both of tyrosine and/or histidine to detrimentally low levels. In some embodiments, a method described within this disclosure comprising delivering PAL activity to a subject further comprises administering L-tyrosine or a metabolic precursor thereof to the subject. In some embodiments, a method described within this disclosure comprising delivering PAL activity to a subject further comprises administering L-histidine or a metabolic precursor thereof to the subject.

A method of treating a metabolic disorder associated with an excess of phenylalanine described within this disclosure may further comprise administering a second agent or therapy to the subject, e.g., in combination with (e.g., prior to, after, or simultaneously with) a host cell, PAL enzyme, or nucleic acid described within this disclosure. In some embodiments, the second agent or therapy is a standard of care treatment for the metabolic disorder. In some embodiments, the second agent or therapy is dietary restriction (e.g., controlling, reducing, or eliminating L-phenylalanine in the subject's diet). In some embodiments, the second agent or therapy is a drug for treating PKU. In some embodiments, the second agent or therapy is a co-factor or activator of PAH. In some embodiments, the second agent or therapy comprises tetrahydrobiopterin or sapropterin dihydrochloride.

In some embodiments, a method of treating a metabolic disorder associated with an excess of phenylalanine described within this disclosure decreases the level of L-phenylalanine in the blood of the subject. In some embodiments, the level of L-phenylalanine in the blood of the subject decreases by at least 10, 20, 30, 40, 50, 60, 70, 80, 90, 95, or 99% relative to pre-treatment or a similar untreated subject.

In some embodiments, a host cell and/or PAL enzyme comprise one or more modifications to enhance their effectiveness (e.g., activity and/or stability (e.g., half-life)) in a selected administration mode. For example, a PAL enzyme may comprise a modification that increases stability and/or activity of the enzyme at acidic pH, e.g., to improve the effectiveness of the PAL enzyme when administered orally and traversing the gastrointestinal tract. In some embodiments, a host cell and/or PAL enzyme comprise one or more modifications to decrease immunogenicity, e.g., to decrease or prevent the subject from forming an immune response to the host cell and/or PAL enzyme. In some embodiments, the PAL enzyme comprises a polyethylene glycol (PEG) molecule. In some embodiments, the PAL enzyme is PEGylated (random covalent ligation of PEG to the enzyme). PEG is known as a non-toxic, non-immunogenic polymer that can improve solubility, decrease immunogenicity, and/or increase half-life in a subject. In some embodiments, the PAL enzyme is immobilized to another agent, e.g., a different enzyme, a polymer (e.g., polysaccharide (e.g., starch)), or an inorganic carrier (e.g., silica gel). Immobilization may increase enzyme stability and/or shelf-life.

The disclosure is also directed, in part, to a method of treating a metabolic disorder associated with an excess of phenylalanine comprising administering to a subject in need thereof a PAL enzyme described within this disclosure or a host cell comprising a PAL enzyme described within this disclosure. Without wishing to be bound by theory, dietary restriction of L-phenylalanine is used to manage PKU, and the disclosure is directed in part to alternative methods of preparing food that is sufficiently low in L-phenylalanine to be suitable for subjects having PKU. In some embodiments, contacting a protein-containing food with a PAL enzyme described within this disclosure (or a host cell comprising the PAL enzyme) decreases the level of L-phenylalanine present in the protein-containing food.

The disclosure is further directed, in part, to a method of treating a metabolic disorder associated with excess tyrosine. In some embodiments, the metabolic disorder associated with excess tyrosine is tyrosinemia. Excess tyrosine in the blood can be caused by deficiencies in the activity of tyrosine metabolizing enzymes. Tyrosinemia is associated with liver cirrhosis, cognitive dysfunctionality, and/or renal failure. In some embodiments, the subject has or is at risk of hepatocellular carcinoma. In some embodiments, a PAL enzyme catalyzes the conversion of L-tyrosine to ammonia and p-coumaric acid. Providing subjects having tyrosinemia or other metabolic disorders associated with an excess of L-tyrosine with the PAL activity of a PAL enzyme described within this disclosure (e.g., by administering a host cell comprising a PAL enzyme described within this disclosure or a nucleic acid encoding the same) may decrease, eliminate, or prevent one or more (e.g., all) symptoms of tyrosinemia or the metabolic disorder.

In some embodiments, a method of treating a metabolic disorder associated with an excess of tyrosine comprises delivering the PAL activity of a PAL enzyme described within this disclosure to a subject in need thereof by a method described within this disclosure.

A method of treating a metabolic disorder associated with an excess of tyrosine described within this disclosure may further comprise administering a second agent or therapy to the subject, e.g., in combination with (e.g., prior to, after, or simultaneously with) a host cell, PAL enzyme, or nucleic acid described within this disclosure. In some embodiments, the second agent or therapy is a standard of care treatment for the metabolic disorder. In some embodiments, the second agent or therapy is dietary restriction (e.g., controlling, reducing, or eliminating L-tyrosine and/or L-phenylalanine in the subject's diet). In some embodiments, the second agent or therapy is a drug for treating tyrosinemia, hepatocellular carcinoma, or both. In some embodiments, the second agent or therapy is nitisinone.

The disclosure is further directed, in part, to a method of treating a cancer. Without wishing to be bound by theory, neoplastic cells are characterized by irregular, often higher metabolic input of amino acids, relative to normal cells, which is thought to be required to support the proliferation of the cancer. In some embodiments, a method of treating cancer comprises administering to a subject a composition described within this disclosure that provides PAL activity (e.g., a PAL enzyme, nucleic acid encoding the same, a host cell comprising a PAL enzyme or nucleic acid encoding the same, or a vector or LNP described within this disclosure). A PAL enzyme may inhibit growth of a tumor by decreasing the available amino acids (e.g., phenylalanine), inhibiting the cancer cells' metabolic activity. In some embodiments, the cancer is selected from breast cancer or prostate cancer.

### Compositions, Kits, and Administration

The present disclosure provides compositions, including pharmaceutical compositions, comprising a host cell described in this application (e.g., a host cell comprising a heterologous polynucleotide encoding a PAL) or one or more enzymes described in this application (e.g., PAL), and optionally a pharmaceutically acceptable excipient.

In certain embodiments, a host cell described in this application is provided in an effective amount in a composition, such as a pharmaceutical composition. In certain embodiments, one or more enzymes described in this application are provided in an effective amount in a composition, such as a pharmaceutical composition. In certain embodiments, the effective amount is a therapeutically effective amount. In certain embodiments, the effective amount is a prophylactically effective amount. In some embodiments, the effective amount is an amount that is sufficient to treat or ameliorate one or more symptoms of hyperphenylalaninemia or phenylketonuria.

In certain embodiments, the subject is an animal. In certain embodiments, the subject is a human. In other embodiments, the subject is a non-human animal. In certain embodiments, the subject is a mammal. In certain embodiments, the subject is a non-human mammal. In some embodiments, the subject is a non-mammal. In certain embodiments, the subject is a domesticated animal, such as a dog, cat, cow, pig, horse, sheep, chicken or goat. In certain embodiments, the subject is a companion animal, such as a dog or cat. In certain embodiments, the subject is a livestock animal, such as a cow, pig, horse, sheep, chicken, or goat. In certain embodiments, the subject is a zoo animal. In another embodiment, the subject is a research animal, such as a rodent (e.g., mouse, rat), dog, pig, or non-human primate.

Compositions, such as pharmaceutical compositions, described in this application can be prepared by any method known in the art. In general, such preparatory methods include bringing a compound described in this application (e.g., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of a pharmaceutical composition comprising a predetermined amount of an active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described in this application will vary, depending upon the identity, size, and/or condition of the subject treated and further depending upon the route by which the composition is to be administered. The composition may comprise, e.g., between 0.1% and 100% (w/w) active ingredient.

The term “pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. Any one of the compositions provided in the present application may include a pharmaceutically acceptable excipient or carrier.

Pharmaceutically acceptable excipients used in the manufacture of pharmaceutical compositions can include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. Exemplary excipients include diluents, dispersing and/or granulating agents, surface active agents and/or emulsifiers, disintegrating agents, binding agents, preservatives, buffering agents, lubricating agents, and/or oils (e.g., synthetic oils, semi-synthetic oils).

The term “pharmaceutically acceptable salt” refers to those salts which are, within the scope of sound medical judgment, suitable for use in contact with the tissues of humans and lower animals without undue toxicity and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, which is incorporated by reference in its entirety. Pharmaceutically acceptable salts of the compounds disclosed in this application include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4- salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Exemplary diluents can include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents can include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers can include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents can include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives can include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants can include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents can include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives can include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives can include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives can include ethanol, polyethylene glycol, phenol, phenolic compounds, bisphenol, chlorobutanol, hydroxybenzoate, and phenylethyl alcohol.

Exemplary acidic preservatives can include vitamin A, vitamin C, vitamin E, beta-carotene, citric acid, acetic acid, dehydroacetic acid, ascorbic acid, sorbic acid, and phytic acid.

Other preservatives can include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents can include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In some embodiments, compositions comprising one or more PALs are formulated for subcutaneous injection. In some embodiments, compositions comprising one or more PALs are formulated for intramuscular injection. Compositions described in this disclosure can be administered via any route that is suitable for the composition and the subject in need thereof.

Injectable preparations, for example sterile injectable aqueous or oleaginous suspensions, can be formulated according to known methods using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed, including synthetic mono- or di-glycerides. In addition, fatty acids such as oleic acid can be used in the preparation of injectables. The injectable formulations can be sterilized, for example, by filtration through a bacterial-retaining filter, or by incorporating sterilizing agents in the form of sterile solid compositions, which can be dissolved or dispersed in sterile water or other sterile injectable medium prior to use.

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

PALs or compositions comprising PALs provided in this application may be formulated in dosage unit form for ease of administration and uniformity of dosage. It will be understood, however, that the total daily usage of the compositions described in this application can be decided by a physician within the scope of sound medical judgment. The specific therapeutically effective dose level for any particular subject or organism will depend upon a variety of factors including the level of toxicity, the age, body weight, general health, and gender of the subject; the time of administration, route of administration, and rate of excretion of the specific active ingredient employed; the duration of the treatment; the PAL used in combination or coincidental with the specific active ingredient employed; and like factors well known in the medical arts.

In some embodiments, the PAL or compositions disclosed in this application are formulated and/or administered in nanoparticles. Nanoparticles are particles in the nanoscale. In some embodiments, nanoparticles are less than 1 μm in diameter. In some embodiments, nanoparticles are between about 1 and 100 nm in diameter. Nanoparticles include organic nanoparticles, such as dendrimers, liposomes, or polymeric nanoparticles. Nanoparticles also include inorganic nanoparticles, such as fullerenes, quantum dots, and gold nanoparticles. Compositions may comprise an aggregate of nanoparticles. In some embodiments, the aggregate of nanoparticles is homogeneous, while in other embodiments the aggregate of nanoparticles is heterogeneous.

The exact amount of a PAL, or composition comprising a PAL, required to achieve an effective amount will vary from subject to subject, depending, for example, on age, and general condition of a subject, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an enzyme described in this application. Dosage forms may be administered at a variety of frequencies. In certain embodiments, when multiple doses are administered to a subject, the frequency of administering the multiple doses to the subject is three doses a day, two doses a day, one dose a day, one dose every other day, one dose every third day, one dose every week, one dose every two weeks, one dose every three weeks, or one dose every four weeks, or less frequent than every four weeks. In certain embodiments, the frequency of administering the multiple doses to the subject is one dose per day. In certain embodiments, the frequency of administering the multiple doses to the is two doses per day. In certain embodiments, the frequency of administering the multiple doses to the subject is three doses per day. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In certain embodiments, the duration between the first dose and last dose of the multiple doses is three months, six months, or one year. In certain embodiments, the duration between the first dose and last dose of the multiple doses is the lifetime of the subject. In some embodiments, dose ranging studies can be conducted to establish optimal therapeutic or effective amounts of the component(s) to be present in dosage forms. In embodiments, the component(s) are present in dosage forms in an amount effective to generate a preventative or therapeutic response to various symptoms of toxicity caused by increased levels of phenylalanine.

Compositions as described in this application can be administered in combination with one or more additional pharmaceutical agents (e.g., therapeutically and/or prophylactically active agents). The compounds or compositions can be administered in combination with additional pharmaceutical agents that improve their activity, improve bioavailability, improve safety, reduce and/or modify metabolism, inhibit excretion, and/or modify distribution in a subject.

Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for reducing levels of phenylalanine, or alleviating the symptoms or toxicity caused by increased levels of phenylalanine. In some embodiments, compositions can be administered concurrently with, prior to, or subsequent to one or more additional pharmaceutical agents.

Also encompassed by the disclosure are kits (e.g., pharmaceutical packs) comprising a composition comprising one or more PALs for use in administering the composition for preventing or reducing increased levels of phenylalanine. The kits provided may comprise a composition, such as a pharmaceutical composition comprising a PAL described in this application, and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or a PAL described in this application. Thus, in one aspect, provided are kits including a container comprising a composition, or PAL described in this application.

In certain embodiments, a kit described in this application further includes instructions for using the kit. A kit may also include information as required by a regulatory agency such as the U.S. Food and Drug Administration (FDA). In certain embodiments, the information included in the kits is prescribing information. A kit may also include one or more additional pharmaceutical agents described in this application as a separate composition.

### EXAMPLES

In order that the invention described in the present application may be more fully understood, the following examples are set forth. The examples described in this application are offered to illustrate the systems and methods provided in this disclosure and are not to be construed in any way as limiting their scope.

### Example 1: Identification of PAL Enzymes with Improved Conversion of Phenylalanine to Trans-Cinnamic Acid

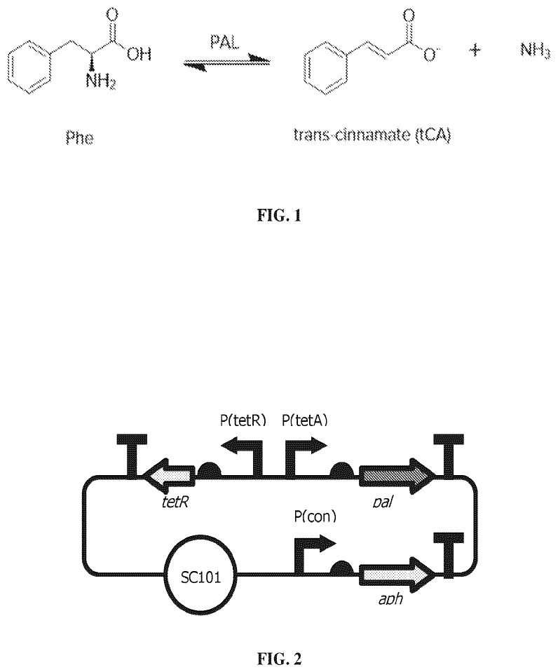

To identify phenylalanine ammonia lyases (PALs, FIG. 1) with improved activity on phenylalanine (Phe) when expressed in Escherichia coli Nissle 1917 (EcN), a library of approximately 1,200 PAL candidate genes was designed from sequences in metagenomic databases with similarity to Photorhabdus laumondii PAL (UniProt Q7N4T3) and Anabeana variabilis PAL (UniProt Q3M5Z3). Protein sequences were encoded in nucleotide sequences using E. coli codon usage and synthesized in the replicative low-copy expression vector shown in FIG. 2. Each candidate enzyme expression construct was transformed into either SYN107 (an E. coli Nissle 1917 (EcN) strain containing a phenylalanine importer PheP integrated into the lacZ locus under control of a tetracycline inducible P(tet) promoter, described in and incorporated by reference from US Patent Publication No. 2017/0312320) or E. coli DH5a. Transformants were selected based on ability to grow on media containing kanamycin at a concentration of 50 mg per liter. A fluorescent protein was included in the library screen as a negative control for enzyme activity. The Photorhabdus laumondii PAL (P1PAL or PAL3; SEQ ID NO: 17) was used as a positive control PAL due to its strong Phe-degrading activity (Isabella et al. Nat. Biotechnol. 36:857-864 (2018) and US Patent Publication No. 2020/0172857, both of which are incorporated by reference within this disclosure in their entireties).

The full set of PALs was first assayed for activity in a primary screen. 647 candidate PALs were screened in SYN107 and 524 were screened in E. coli DH5a. Whole cell and cell lysate assays were performed to screen the libraries. The PAL reaction (FIG. 1) releases tCA as the product, and tCA production can be determined in a plate reader by measuring absorbance at 290 nm in supernatant and cell lysate samples. For the whole-cell assay screens, the strains expressing the PAL library were induced with 200 ng/ml anhydrotetracycline in LB media and stored as glycerol stocks in phenylketonuria (PKU) phosphate formulation buffer at −80° C. At the time of the PKU assay, the strains were added to an OD600=˜1.0 and incubated for 30-60 min with 40 mM Phe in an M9+0.5% glucose medium at room temperature. The plates were centrifuged, supernatant was transferred to UV-transparent 96-well plates, and A290 measurements were taken. For the cell lysate assay, the PAL library was induced as for the whole-cell assay. The pellets were resuspended in a Bugbuster solution supplemented with 25 U/mL rLysozyme and 3000 U/mL universal nuclease and incubated for 30 min at room temperature. The resulting lysates were then added to assay buffer, resulting in a reaction with a final composition of 50 mM Tris pH 7.5, 1 mM Phe and 5% Bugbuster. Absorbance readings at 290 nm were taken over a period of 5 min and the tCA production rate was reported in μM min-1 OD600-1 or mA290 min-1 OD600-1. The primary screening was conducted at room temperature.

49 SYN107-based library strains with a lysate normalized rate >1.7 and/or a whole tCA/OD ratio >0.2 were advanced to secondary screening. In the E. coli DHSalpha primary screen, six strains exceeded the positive control P1PAL mean in both cell lysate and whole cell OD-normalized activity. Two strains were found to be strikingly better than the positive control: t732452 and t732438, which expressed an Anabaena variabilis PAL (AvPAL; SEQ ID NO: 3) and a maltose binding protein-Capsicum annuum (Capsicum pepper) PAL fusion (SEQ ID NO: 19), respectively.

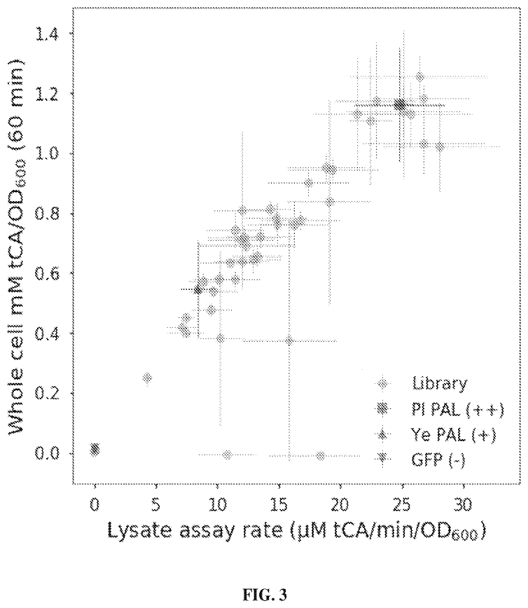

A secondary screen was performed to investigate the reproducibility of the 49 active SYN107-derived PAL transformants. As for the primary screen, lysate and whole-cell assays were performed. The whole-cell assay was performed at 37° C., and timepoints were taken at 30 and 60 min. The 60 min tCA production data from the lysate and whole cell assay are shown in FIG. 3. Several PALs again demonstrated comparable activity to the positive control P1PAL in the assay, although none was evidently better than the positive control based on this analysis.

To select PALs for tertiary screening, two criteria were used. First, the PALs with the best reproducible OD-normalized activity in cell lysate and whole-cell assays were selected. Second, some PALs were selected that were predicted to exhibit low expression as determined by translation initiation rate (TIR) calculations (Farasat et al. Mol. Cyst. Biol. 10:73 (2014)), yet still exhibited good activity. The TIRs were used to calculate a TIR-adjusted and OD600-normalized tCA production, and high values suggested that the PAL was more active than appeared from the tCA per OD calculation. In total, 8 PALs were advanced to tertiary screening (Table 2). One additional PAL, AvPAL M222L (Mays et al. Chem. Commun. 56:5255-5258 (2020)), was added as an additional positive control.

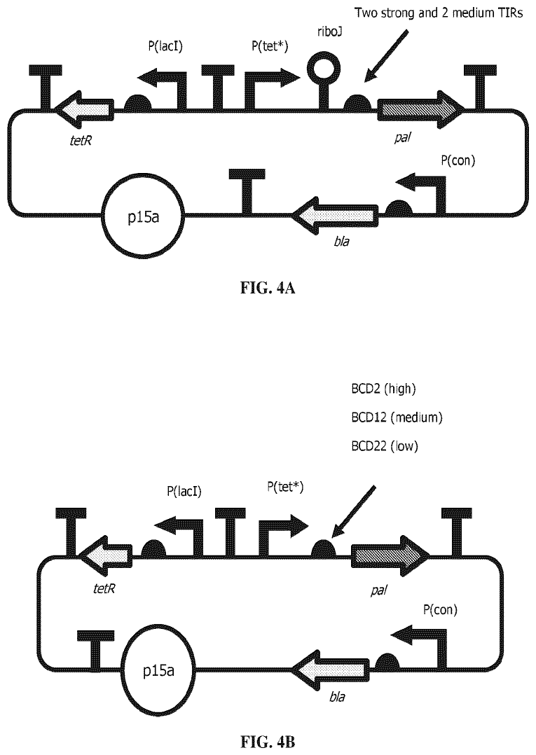

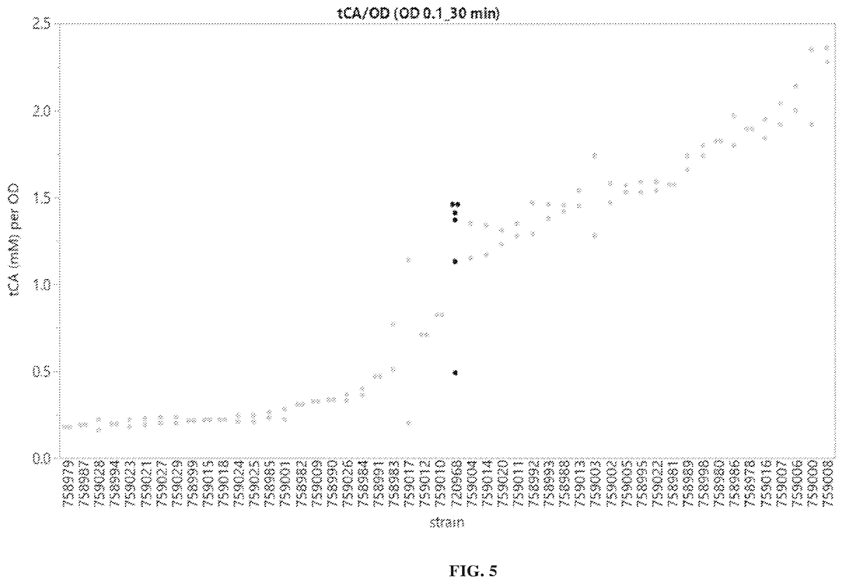

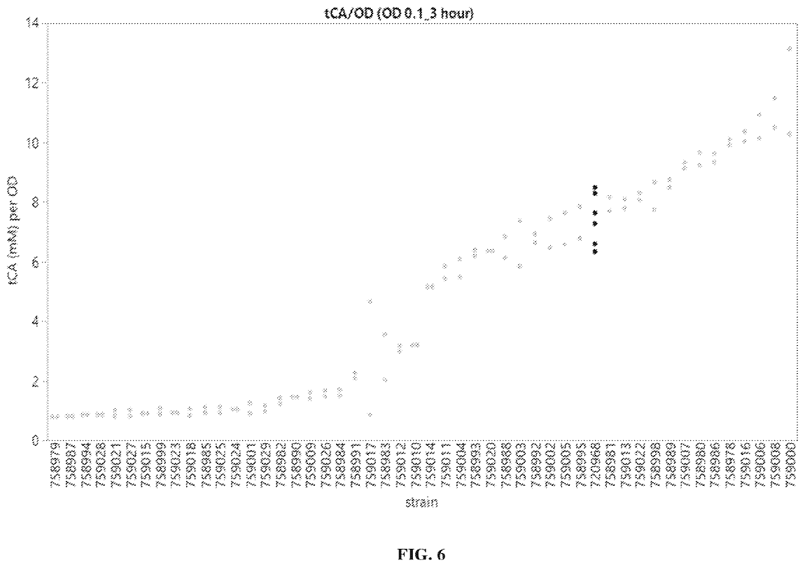

The 9 PALs were cloned into p15a vectors under the control of a P(tet*) promoter with two RBS strategies. In one vector, three bicistronic designs (BCDs) were used to test different PAL expression levels. These were BCD2 for high expression, BCD12 for medium expression and BCD22 for low expression (Mutalik et al. Nat. Methods 10:354 (2013)). In the other vector, the transcriptional insulator riboJ (Lou et al. Nat. Biotechnol. 30:1137 (2012)) was used upstream of selected TIRs of ˜1e5 and −1e3 AUs, with 1e5 and 1e4 associated with high gene expression and 1e3 associated with medium gene expression (FIGS. 4A-4B). These were then screened in the PKU potency whole-cell assay described above with the incubation and assay steps performed at a temperature of 37° C. The tCA production activity was determined at cell density of OD600=0.1 at a 30 min timepoint (FIG. 5) and a 3 hour timepoint (FIG. 6). Surprisingly, for some enzymes, higher predicted gene expression did not correlate to increased tCA production activity.

Three PALs demonstrated >40% improvement over the positive control P1PAL enzyme in the tertiary screen (Table 3). One was the control AvPAL M222L mutant enzyme (Mays et al. Chem. Commun. 56:5255-5258 (2020)) and the other two were enzymes derived from plants: Gossypium raimondii (New World cotton) PAL (GrPAL; SEQ ID NO: 1) and Vitis vinifera (Grape) PAL (VvPAL; SEQ ID NO: 15). These plant PAL enzymes are considerably longer than bacterial PALs; GrPAL and VvPAL are 720 and 707 amino acids in length, respectively, whereas the bacterial AvPAL and the positive control P1PAL enzymes are 567 and 532 amino acids in length, respectively. The bacterial and plant PALs also share low sequence identity; e.g., GrPAL only has 31.0 and 34.2% identity to P1PAL and AvPAL, respectively, over the aligned amino acid residues, and VvPAL has only has 30.4 and 33.1% identity to P1PAL and AvPAL, respectively, over the aligned amino acid residues, as determined using Clustal Omega (Sievers et al., Mol Syst Biol. 2011 Oct. 11; 7:539) using default parameters. Therefore, these plant-derived enzymes represent a new group of distantly related PALs that show potential for the treatment of PKU.

Table 2 shows the 8 PALs selected for the PAL tertiary screen selected from the primary and secondary screening described above. The P1PAL positive control is also included.

TABLE 2
Top PALs selected for the PAL tertiary screen

| | | | | | Expression- and |
|---|---|---|---|---|---|
| | | | | | OD600- adjusted |
| | | | | tCA per OD | tCA production |
| Strain | SEQ ID | | TIR | [mM/ | [tCA per OD]/ |
| ID | NO: | PAL source | [AU] | OD600] | TIR] *1000 |
| t727023 | 1 | Gossypium | 733 | 0.59 | 0.81 |
| | | raimondii (New | | | |
| | | World cotton) | | | |
| | | GrPAL | | | |
| t726062 | 3 | Anabaena variabilis | 2.5e3 | 0.29 | 0.11 |
| | | AvPAL | | | |
| t726692 | 5 | Rhizobium | 158 | 0.45 | 2.82 |
| | | radiobacter RrPAL | | | |
| t731343 | 7 | Arabidopsis thaliana | 83 | 0.51 | 6.19 |
| | | AtPAL2 | | | |
| t732438 | 19 | Capsicum annuum | 2.6e3 | 1.34 | 0.51 |
| | | (Capsicum pepper) | | | |
| | | MBP-CaPAL | | | |
| t732611 | 11 | Salvia miltiorrhiza | 3.4 | 0.026 | 7.51 |
| | | (Chinese sage) | | | |
| | | SmPAL | | | |
| t726556 | 13 | Ricinus communis | 1.4e3 | 0.50 | 0.35 |
| | | (Castor bean) | | | |
| | | RcPAL | | | |
| t732247 | 15 | Vitis vinifera | 146 | 0.28 | 1.91 |
| | | (Grape) VvPAL | | | |
| t720968 | 17 | Photorhabdus | 2.4e3 | 0.89 | 0.36 |
| | | luminescens subsp. | | | |
| | | laumondii PlPAL | | | |

Table 3 shows the tertiary screen hit strains with activity >10% higher than the P1PAL positive control expression strain t758992 (expressing the P4PAL control on an p15a vector bearing an RBS predicted to have a strong RBS). The strains also had better activity than the strain t720968 (expressing the P1PAL control on an SC101 vector) used in the primary and secondary screens.

TABLE 3
Top hits from the tertiary screen (compared to positive control strain t758992)

| | Theoretical | | | | | | |
|---|---|---|---|---|---|---|---|
| | Strength | | | Ave tCA | Std | % improved | % improved |
| Strain | of | | | (mM)/OD, | dev, 30 | to t758992, | to t758992, |
| ID | Expression | RBS | PAL | 30 min TP | min TP | 30 min TP | 3 h TP |
| t759008 | Medium | TIR | AvPAL | 2.32 | 0.06 | 74.0 | 43.3 |
| | | 3.1e3 AU | M222L | | | | |
| t759000 | High | TIR | GrPAL | 2.14 | 0.30 | 60.1 | 52.7 |
| | | 1.9e4 AU | | | | | |
| t759006 | High | TIR | AvPAL | 2.07 | 0.10 | 55.3 | 37.4 |
| | | 7.7e4 AU | M222L | | | | |
| t759007 | Medium | TIR | AvPAL | 1.98 | 0.08 | 48.5 | 19.5 |
| | | 5.8e4 AU | M222L | | | | |
| t758978 | High | TIR | VvPAL | 1.90 | 0.01 | 42.1 | 30.0 |
| | | 5.3e4 AU | | | | | |
| t759016 | High | BCD2 | VvPAL | 1.90 | 0.08 | 42.1 | 33.1 |
| t758986 | High | TIR | RcPAL | 1.89 | 0.12 | 41.4 | 23.7 |
| | | 7.8e4 AU | | | | | |
| t758980 | Medium | TIR | VvPAL | 1.83 | 0.01 | 36.9 | 23.1 |
| | | 2.9e3 AU | | | | | |
| t758998 | High | TIR | GrPAL | 1.77 | 0.04 | 32.8 | 7.0 |
| | | 2.9e4 AU | | | | | |
| t758989 | High | TIR | RcPAL | 1.70 | 0.06 | 27.5 | 12.4 |
| | | 1.2e5 AU | | | | | |
| t758981 | Medium | TIR | VvPAL | 1.58 | 0.01 | 18.1 | 3.4 |
| | | 1.8e3 AU | | | | | |
| t759022 | Medium | BCD12 | VvPAL | 1.57 | 0.04 | 17.4 | 6.8 |
| t758995 | Medium | TIR | mbp- | 1.56 | 0.04 | 17.0 | −4.6 |
| | | 1.8e3 AU | CaPAL | | | | |
| t759005 | Medium | TIR | AvPAL | 1.55 | 0.03 | 16.3 | −7.4 |
| | | 1.8e3 AU | | | | | |
| t759002 | Medium | TIR | AvPAL | 1.53 | 0.08 | 14.4 | −9.3 |
| | | 8.2e4 AU | | | | | |
| t759003 | High | TIR | AvPAL | 1.51 | 0.33 | 13.3 | −13.8 |
| | | 6.8e4 AU | | | | | |
| t759013 | Medium | TIR | AtPAL2 | 1.50 | 0.06 | 12.1 | 3.6 |
| | | 1.6e3 AU | | | | | |

### Example 2: Engineering of the Photorhabdus laumondii PAL Enzyme for Improved Conversion of Phenylalanine to Trans-Cinnamic Acid

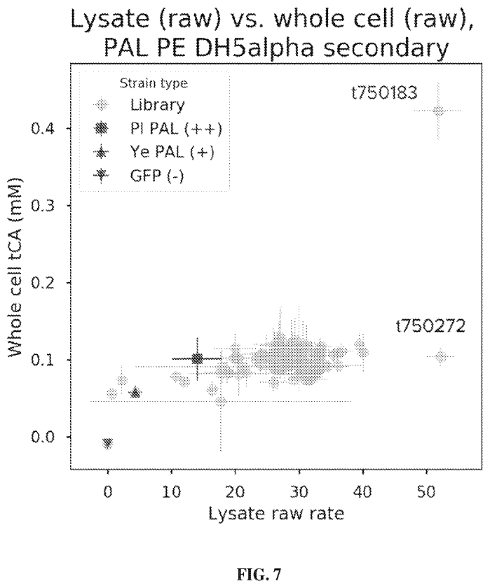

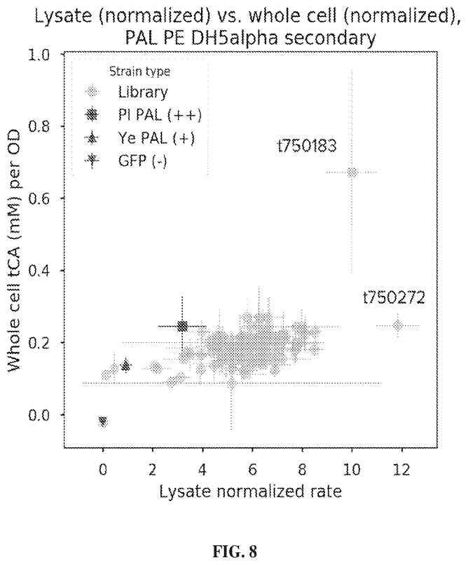

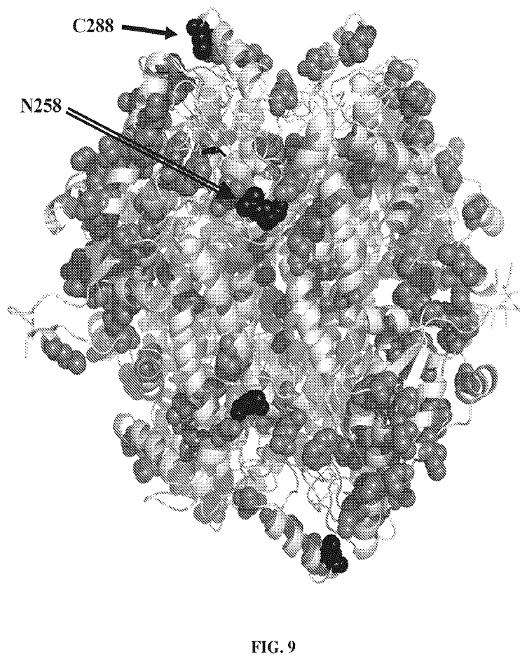

A homology model of P1PAL was generated using comparative modeling using the AvPAL crystal structure (PDBid: 2NYN & 5LTM) as a template. Saturation mutagenesis was performed in the active site, defined for this purpose as any non-catalytically essential residue position with a Calpha atom within 8.5 Angstroms of the docked L-Phe substrate in the P1PAL model. In addition, EVcouplings analysis ((Hopf et al. Bioinformatics 35:1582-1584 (2019), Hopf et al. Nature Biotech. 35, 128-135 (2017)) was conducted to identify favorable mutations throughout the protein. 858 of these P1PAL variants were synthesized in the replicative low-copy expression vector shown in FIG. 2 and transformed into E. coli DH5a. These strains were screened in both whole cell and cell lysate format as described in Example 1. The top 100 performing library strains were advanced to a secondary screen. One PAL was found to be ˜1.7× improved in the whole cell assay, whether normalized to cell density or not. Most of the P1PAL mutants advanced from the primary screen had improved activity over the P1PAL wild-type protein in the cell lysate assay, and one standout P1PAL mutant was present that had ˜3.7× improvement in the OD-normalized or unadjusted rate (FIG. 7, FIG. 8). The top PAL whole cell assay hit contained an N258R mutation which lies >20 angstroms from the active site. The standout cell lysate hit contained a C288S mutation that was also distant to the active site. The N258 and C288 residues are predicted to either be adjacent to or be a part of a flexible outer loop which is proposed to play a role in the regulation of substrate binding (Bata, 2019, “Investigation of structure-function relationship in hydroxynitrile lyases and MIO-containing class I lyase like enzymes”, Thesis Booklet, Budapest University of Technology and Economics, p. 28-32). Surprisingly, most of the other hits were also >20 angstroms away from the active site with the ones substantially improving activity occurring on the protein surface (FIG. 9).

A second engineered library was designed using P1PAL and AvPAL as the templates. Combinations of point mutations were generated. Another round of structure-informed design was carried out focusing on hotspots identified in the first two library screens. AvPAL mutants were also generated based on EVcouplings analysis and structure-based protein engineering using the AvPAL high resolution crystal structure PDBid: 5LTM.

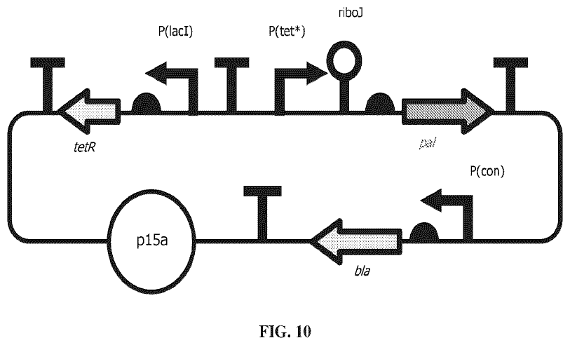

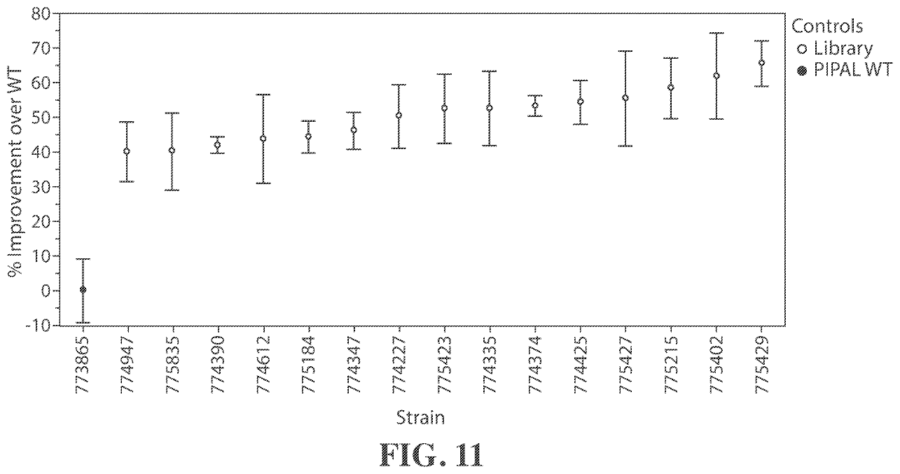

In total, ˜1600 PALs were screened including mutants from the two PAL templates (˜450 AvPAL library strains and ˜1150 P1PAL mutant strains) on a p15a-P(tet*) vector (FIG. 10). The library was transformed into SYN107 and screened in the whole cell PKU potency assay as described in Example 1 with an assay incubation period of 3 hours at 37° C. 100 mg/L carbenicillin was used in place of kanamycin for growing the strains and selecting for transformants. Out of the AvPAL library strains that were screened, the best hit demonstrated ˜2.4× improved activity over the WT AvPAL strain. The best AvPAL mutant in the whole cell assay, G218A, demonstrated ˜145% or better improved activity over both the previously reported M222L and L4P G218S evolved AvPAL mutants. Out of the P1PAL mutants that were screened, the best hit demonstrated activity >1.5× of WT P1PAL.

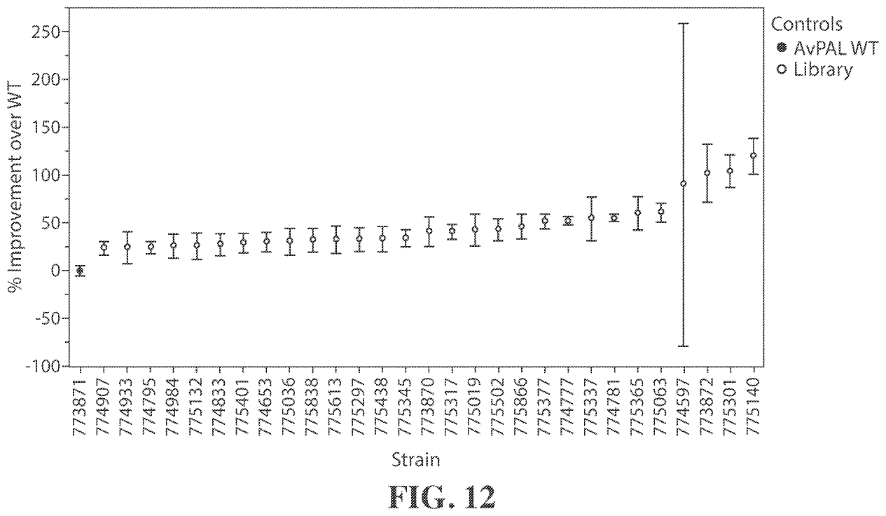

The top 108 P1PAL and 83 AvPAL mutant strains were selected for a secondary screen. The strains were screened in quadruplicate and whole cell assays were performed. As expected from the primary screen, many P1PAL mutant strains demonstrated better activity than WT P1PAL, with the best performer demonstrating 1.6× improvement relative to WT P1PAL (FIG. 11, Table 4 and Table 5). Most of the 83 AvPAL mutant strains also demonstrated improvement over WT as expected from the primary screen, with the strain bearing the G218A mutant identified again as the top performing strain (FIG. 12, Table 6 and Table 7).

Table 4 shows the secondary screen whole cell assay data from select strains exhibiting >10% improved activity compared to the P1PAL positive control expression strain t773865 (P1PAL on an p15a vector).

TABLE 4
Data from secondary screen whole cell assay (compared to positive control t773865)

| | Template | | Ave tCA | Std dev | % Improved to |
|---|---|---|---|---|---|
| Strain | SEQ ID | Mutation (relative to | (mM)/ | of tCA/ | in-plate t773865 |
| ID | NO: | Template) | OD600 | OD600 | (WT PlPAL) |
| t774947 | 17 | S92G H133M I167K | 1.015 | 0.063 | 40.1 |
| | | V470A A263T T40I | | | |
| t775429 | 17 | S92G L432I V470A T40I | 1.015 | 0.041 | 65.6 |
| t775402 | 17 | S92G V470A M111V T40I | 0.993 | 0.075 | 62.1 |
| t774227 | 17 | C288S T40I I502V S499P | 0.989 | 0.061 | 50.3 |
| t775215 | 17 | S92G L432I V470A N258R | 0.972 | 0.054 | 58.5 |
| | | T40I | | | |
| t775427 | 17 | S92G N258R C288S T40I | 0.953 | 0.084 | 55.6 |
| t774923 | 17 | S92G L432I V470A N258R | 0.953 | 0.026 | 31.5 |
| | | M111V | | | |
| t775423 | 17 | S92G V470A A263T T40I | 0.935 | 0.061 | 52.5 |
| t775835 | 17 | S92G V470A A433S | 0.919 | 0.073 | 40.2 |
| | | N258R T40I | | | |
| t774425 | 17 | S92G L432I V470A H133F | 0.919 | 0.038 | 54.4 |
| | | N258I | | | |
| t774374 | 17 | S92G L432I M111V T40I | 0.912 | 0.017 | 53.3 |
| t775535 | 17 | S92G V470A A433S N258I | 0.911 | 0.045 | 25.7 |
| | | T40I | | | |
| t774335 | 17 | N258I T40I I502V S499P | 0.908 | 0.064 | 52.6 |
| t774837 | 17 | S92G L432I V470A A263T | 0.901 | 0.099 | 37.6 |
| | | M111V | | | |
| t775114 | 17 | S92G H133M I167K | 0.897 | 0.056 | 26.5 |
| | | V470A A433S T40I | | | |
| t775330 | 17 | S92G V470A H133F | 0.890 | 0.091 | 22.8 |
| | | N258R T40I | | | |
| t775332 | 17 | S92G H133F A433S C288S | 0.887 | 0.013 | 22.4 |
| t775184 | 17 | S92G V470A A433S | 0.885 | 0.028 | 44.4 |
| | | N275S | | | |
| t775256 | 17 | S92G H133F N258R C288S | 0.880 | 0.033 | 21.5 |
| t775642 | 17 | S92G H133F N258R T40I | 0.878 | 0.036 | 33.4 |
| t775322 | 17 | M111V N275S I502V | 0.877 | 0.017 | 21.0 |
| | | S499P | | | |
| t775138 | 17 | S92G V470A A433S | 0.876 | 0.097 | 23.6 |
| | | C288S T40I | | | |
| t775439 | 17 | S92G L432I H133F N258I | 0.874 | 0.070 | 20.6 |
| | | T40I | | | |
| t775143 | 17 | S92G V470A N258I | 0.871 | 0.103 | 32.9 |
| t774347 | 17 | S92G L432I V470A N258I | 0.870 | 0.033 | 46.2 |
| | | T40I | | | |
| t775519 | 17 | L432I V470A N258R | 0.865 | 0.010 | 19.4 |
| | | M111V | | | |
| t774615 | 17 | C288S I502V S499P | 0.862 | 0.075 | 35.1 |
| t775326 | 17 | V470A A433S C288S T40I | 0.860 | 0.017 | 18.6 |
| t775656 | 17 | S92G L432I N258R | 0.860 | 0.056 | 30.6 |
| | | M111V T40I | | | |
| t775921 | 17 | S92G V470A A433S | 0.858 | 0.072 | 31.0 |
| | | C288S | | | |
| t774612 | 17 | H133M I167K L432I N258I | 0.856 | 0.076 | 43.8 |
| | | T40I | | | |
| t774888 | 17 | H133M I167K V470A | 0.855 | 0.029 | 30.5 |
| | | N258I | | | |
| t775798 | 17 | S92G V470A N275S | 0.853 | 0.084 | 30.2 |
| t775026 | 17 | S92G L432I C288S T40I | 0.851 | 0.116 | 20.0 |
| t775350 | 17 | S92G L432I H133F T40I | 0.846 | 0.032 | 16.8 |
| t774419 | 17 | S92G V470A A433S | 0.845 | 0.057 | 28.4 |
| | | N258R | | | |
| t774390 | 17 | S92G L432I V470A | 0.845 | 0.014 | 42.0 |
| | | M111V T40I | | | |
| t774249 | 17 | S92G H133M I167K L432I | 0.845 | 0.058 | 28.3 |
| | | V470A T40I | | | |
| t775099 | 17 | S92G V470A H133F | 0.844 | 0.076 | 32.3 |
| t775211 | 17 | S92G L432I M111V N258I | 0.840 | 0.064 | 16.0 |
| t774546 | 17 | S92G L432I V470A N258R | 0.837 | 0.052 | 31.1 |
| t775505 | 17 | S92G V470A H133F | 0.831 | 0.084 | 35.6 |
| | | A433S C288S | | | |
| t774222 | 17 | S92G V470A M111V | 0.830 | 0.075 | 39.5 |
| | | N258I T40I | | | |
| t775566 | 17 | V470A A433S N258R T40I | 0.830 | 0.060 | 26.1 |
| t774414 | 17 | S92G H133M I167K | 0.830 | 0.060 | 39.4 |
| | | V470A A433S N258R | | | |
| t774972 | 17 | S92G V470A H133F | 0.824 | 0.078 | 29.1 |
| | | A433S T40I | | | |
| t775821 | 17 | S92G C288S M111V | 0.823 | 0.049 | 25.0 |
| t775853 | 17 | S92G V470A H133F | 0.822 | 0.031 | 24.9 |
| | | A433S N258I | | | |
| t773973 | 17 | V470A A433S M111V | 0.821 | 0.069 | 38.1 |
| | | T40I | | | |
| t775476 | 17 | S92G C288S | 0.821 | 0.099 | 20.5 |
| t775813 | 17 | S92G H133F A433S C288S | 0.817 | 0.093 | 24.2 |
| | | T40I | | | |
| t774309 | 17 | S92G L432I V470A H133F | 0.816 | 0.020 | 37.1 |
| | | N258R | | | |
| t773984 | 17 | H133M I167K V470A | 0.815 | 0.069 | 37.0 |
| | | A433S N258R T40I | | | |
| t774549 | 17 | S92G V470A A263T | 0.814 | 0.079 | 27.7 |
| | | M111V T40I | | | |
| t775120 | 17 | V470A A433S A263T | 0.812 | 0.061 | 14.6 |
| | | M111V | | | |
| t775760 | 17 | S92G L432I V470A | 0.812 | 0.100 | 23.4 |
| | | M111V N258I | | | |
| t775950 | 17 | S92G V470A A433S | 0.811 | 0.039 | 19.0 |
| t774169 | 17 | S92G H133M I167K L432I | 0.810 | 0.079 | 36.2 |
| | | V470A N258R | | | |
| t774849 | 17 | S92G V470A | 0.809 | 0.103 | 26.8 |
| t775271 | 17 | S92G N258R | 0.806 | 0.023 | 11.2 |
| t775629 | 17 | V470A N258R T40I | 0.804 | 0.086 | 39.2 |
| t775815 | 17 | H133M I167K V470A | 0.804 | 0.086 | 22.1 |
| | | A433S T40I | | | |
| t775562 | 17 | H133M I167K V470A | 0.802 | 0.087 | 21.9 |
| | | N258R T40I | | | |
| t774455 | 17 | S92G V470A N258R | 0.802 | 0.053 | 34.7 |
| | | M111V T40I | | | |
| t775633 | 17 | S92G V470A H133F | 0.800 | 0.052 | 17.5 |
| | | C288S | | | |
| t774662 | 17 | S92G L432I V470A H133F | 0.799 | 0.099 | 25.3 |
| | | A263T | | | |
| t773864 | 17 | S92G V470A N258R | 0.798 | 0.104 | 23.8 |
| | | N275S | | | |
| t774921 | 17 | H133M I167K V470A | 0.794 | 0.071 | 12.0 |
| | | N258I T40I | | | |
| t774218 | 17 | S92G V470A A433S | 0.778 | 0.027 | 30.8 |
| | | M111V T40I | | | |
| t774339 | 17 | S92G V470A N258R | 0.777 | 0.026 | 18.0 |
| t775948 | 17 | S92G H133M I167K L432I | 0.776 | 0.098 | 13.9 |
| | | V470A N258I | | | |
| t773867 | 17 | C288S | 0.773 | 0.058 | 20.6 |
| t775602 | 17 | S92G L432I H133F | 0.771 | 0.075 | 17.2 |
| t774660 | 17 | S92G N258R C288S | 0.770 | 0.064 | 20.8 |
| | | M111V T40I | | | |
| t775339 | 17 | N258R N275S M402L | 0.761 | 0.082 | 31.8 |
| t774332 | 17 | V470A T40I | 0.757 | 0.036 | 27.3 |
| t775788 | 17 | S92G H133M I167K | 0.748 | 0.091 | 13.6 |
| | | V470A A263T | | | |
| t775315 | 17 | S92G V470A A433S | 0.746 | 0.057 | 29.3 |
| | | N258R N275S | | | |
| t775111 | 17 | A433S N258R C288S T40I | 0.743 | 0.072 | 13.5 |
| t775103 | 17 | S92G A433S M111V | 0.742 | 0.073 | 16.2 |
| | | N258I | | | |
| t774299 | 17 | N258I I502V S499P | 0.741 | 0.026 | 24.6 |
| t775692 | 17 | S92G L432I V470A A263T | 0.736 | 0.078 | 36.2 |
| | | T40I | | | |
| t775698 | 17 | V470A N258R | 0.732 | 0.077 | 14.7 |
| t774782 | 17 | S92G H133M I167K | 0.728 | 0.060 | 26.1 |
| | | V470A N258I T40I | | | |
| t775734 | 17 | S92G H133M I167K | 0.726 | 0.035 | 13.8 |
| | | V470A N258I | | | |
| t775806 | 17 | V470A M111V | 0.725 | 0.076 | 13.6 |
| t774731 | 17 | N258I T40I M402L | 0.719 | 0.059 | 12.6 |
| t775335 | 17 | N258R T40I M402L | 0.708 | 0.024 | 22.5 |
| t775343 | 17 | V470A A433S | 0.685 | 0.078 | 18.5 |
| t775598 | 17 | H133M I167K V470A | 0.682 | 0.023 | 26.1 |
| | | C288S T40I | | | |
| t775375 | 17 | S92G H133M I167K | 0.680 | 0.054 | 17.8 |
| | | V470A A433S A263T | | | |
| | | C288S | | | |
| t774639 | 17 | H133M I167K V470A | 0.672 | 0.039 | 24.2 |
| | | A433S | | | |
| t775590 | 17 | S92G V470A N258R | 0.670 | 0.038 | 23.9 |
| | | C288S | | | |
| t775558 | 17 | V470A H133F | 0.665 | 0.036 | 23.0 |

Table 5 shows the secondary screen cell lysate assay data of select strains exhibiting >10% improved activity compared to the P1PAL positive control expression strain t773865 (P1PAL on an p15a vector).

TABLE 5
Data from secondary screen cell lysate assay (compared to positive control t773865)

| | | | Ave OD | Std dev of OD | |
|---|---|---|---|---|---|
| | | | normalized | normalized | % Improved to |
| | Template | | rate | rate | in-plate |
| Strain | SEQ ID | Mutation (relative to | (mA290/min/ | (mA290/min/ | t773865 |
| # | NO: | Template) | OD600) | OD600) | (WT PlPAL) |
| t774837 | 17 | S92G L432I V470A A263T | 37.8 | 2.68 | 127.2 |
| | | M111V | | | |
| t774660 | 17 | S92G N258R C288S | 36.7 | 1.31 | 102.0 |
| | | M111V T40I | | | |
| t775921 | 17 | S92G V470A A433S | 36.1 | 1.86 | 116.7 |
| | | C288S | | | |
| t775798 | 17 | S92G V470A N275S | 35.5 | 3.84 | 113.4 |
| t775633 | 17 | S92G V470A H133F | 35.1 | 2.84 | 283.2 |
| | | C288S | | | |
| t775806 | 17 | V470A M111V | 35.0 | 1.93 | 92.7 |
| t775111 | 17 | A433S N258R C288S T40I | 33.1 | 1.69 | 99.1 |
| t774615 | 17 | C288S I502V S499P | 33.1 | 7.93 | 81.8 |
| t773867 | 17 | C288S | 33.0 | 4.46 | 95.5 |
| t775099 | 17 | S92G V470A H133F | 32.8 | 3.94 | 80.6 |
| t775941 | 17 | S92G H133M I167K | 32.8 | 3.64 | 258.2 |
| | | A433S N258R C288S | | | |
| t774662 | 17 | S92G L432I V470A H133F | 32.4 | 2.26 | 78.4 |
| | | A263T | | | |
| t774549 | 17 | S92G V470A A263T | 32.1 | 3.61 | 76.5 |
| | | M111V T40I | | | |
| t774923 | 17 | S92G L432I V470A N258R | 31.8 | 9.54 | 78.6 |
| | | M111V | | | |
| t775339 | 17 | N258R N275S M402L | 31.2 | 1.30 | 95.6 |
| t775371 | 17 | S92G L432I V470A N258R | 31.1 | 9.59 | 238.9 |
| | | C288S | | | |
| t775315 | 17 | S92G V470A A433S | 30.7 | 0.79 | 92.7 |
| | | N258R N275S | | | |
| t775347 | 17 | S92G A433S C288S N258I | 30.4 | 1.79 | 90.7 |
| t774390 | 17 | S92G L432I V470A | 29.8 | 0.84 | 157.1 |
| | | M111V T40I | | | |
| t775143 | 17 | S92G V470A N258I | 29.3 | 1.39 | 76.0 |
| t775375 | 17 | S92G H133M I167K | 29.1 | 2.14 | 82.6 |
| | | V470A A433S A263T | | | |
| | | C288S | | | |
| t773864 | 17 | S92G V470A N258R | 28.7 | 3.21 | 85.0 |
| | | N275S | | | |
| t775734 | 17 | S92G H133M I167K | 28.6 | 0.87 | 57.4 |
| | | V470A N258I | | | |
| t774347 | 17 | S92G L432I V470A N258I | 28.4 | 2.20 | 144.3 |
| | | T40I | | | |
| t775476 | 17 | S92G C288S | 28.2 | 5.51 | 208.1 |
| t774455 | 17 | S92G V470A N258R | 28.0 | 2.81 | 141.5 |
| | | M111V T40I | | | |
| t775821 | 17 | S92G C288S M111V | 27.8 | 8.21 | 194.1 |
| t774888 | 17 | H133M I167K V470A | 27.7 | 1.57 | 66.4 |
| | | N258I | | | |
| t775835 | 17 | S92G V470A A433S | 27.5 | 2.41 | 65.5 |
| | | N258R T40I | | | |
| t774546 | 17 | S92G L432I V470A N258R | 27.4 | 1.23 | 50.8 |
| t775215 | 17 | S92G L432I V470A N258R | 27.3 | 3.52 | 97.8 |
| | | T40I | | | |
| t774972 | 17 | S92G V470A H133F | 27.3 | 1.21 | 50.1 |
| | | A433S T40I | | | |
| t775322 | 17 | M111V N275S I502V | 27.2 | 5.98 | 52.7 |
| | | S499P | | | |
| t774222 | 17 | S92G V470A M111V | 27.2 | 6.40 | 134.0 |
| | | N258I T40I | | | |
| t775948 | 17 | S92G H133M I167K L432I | 27.1 | 2.84 | 195.7 |
| | | V470A N258I | | | |
| t773973 | 17 | V470A A433S M111V | 26.2 | 2.09 | 126.0 |
| | | T40I | | | |
| t775399 | 17 | L432I N275S | 26.0 | 8.44 | 184.0 |
| t775103 | 17 | S92G A433S M111V | 25.7 | 5.12 | 41.5 |
| | | N258I | | | |
| t774425 | 17 | S92G L432I V470A H133F | 25.5 | 3.35 | 119.9 |
| | | N258I | | | |
| t774227 | 17 | C288S T40I I502V S499P | 25.4 | 0.40 | 168.8 |
| t775343 | 17 | V470A A433S | 25.3 | 1.44 | 58.5 |
| t774374 | 17 | S92G L432I M111V T40I | 25.2 | 3.17 | 116.7 |
| t775332 | 17 | S92G H133F A433S C288S | 25.0 | 6.62 | 40.1 |
| t774782 | 17 | S92G H133M I167K | 24.6 | 1.19 | 54.2 |
| | | V470A N258I T40I | | | |
| t774309 | 17 | S92G L432I V470A H133F | 24.0 | 2.16 | 106.7 |
| | | N258R | | | |
| t774218 | 17 | S92G V470A A433S | 23.8 | 2.91 | 104.7 |
| | | M111V T40I | | | |
| t774335 | 17 | N258I T40I I502V S499P | 23.5 | 6.49 | 102.2 |
| t774731 | 17 | N258I T40I M402L | 23.1 | 0.63 | 26.8 |
| t775698 | 17 | V470A N258R | 23.0 | 5.36 | 26.5 |
| t775427 | 17 | S92G N258R C288S T40I | 22.7 | 7.59 | 64.3 |
| t775629 | 17 | V470A N258R T40I | 22.6 | 1.20 | 41.6 |
| t775138 | 17 | S92G V470A A433S | 22.5 | 8.30 | 181.9 |
| | | C288S T40I | | | |
| t775760 | 17 | S92G L432I V470A | 22.4 | 5.05 | 137.1 |
| | | M111V N258I | | | |
| t775326 | 17 | V470A A433S C288S T40I | 22.0 | 6.14 | 23.5 |
| t775211 | 17 | S92G L432I M111V N258I | 21.8 | 5.11 | 22.6 |
| t775396 | 17 | S92G M111V N275S | 21.7 | 7.23 | 21.9 |
| t774849 | 17 | S92G V470A | 21.7 | 4.37 | 19.5 |
| t775950 | 17 | S92G V470A A433S | 21.6 | 2.58 | 135.6 |
| t774612 | 17 | H133M I167K L432I N258I | 21.5 | 0.82 | 85.5 |
| | | T40I | | | |
| t775598 | 17 | H133M I167K V470A | 21.4 | 0.49 | 126.9 |
| | | C288S T40I | | | |
| t774919 | 17 | S92G L432I H133F N258R | 21.4 | 3.11 | 168.1 |
| | | C288S | | | |
| t774332 | 17 | V470A T40I | 21.2 | 1.73 | 82.2 |
| t775256 | 17 | S92G H133F N258R C288S | 21.0 | 0.90 | 18.0 |
| t773984 | 17 | H133M I167K V470A | 21.0 | 0.99 | 80.8 |
| | | A433S N258R T40I | | | |
| t775535 | 17 | S92G V470A A433S N258I | 20.3 | 6.42 | 13.9 |
| | | T40I | | | |
| t775590 | 17 | S92G V470A N258R | 20.0 | 0.95 | 112.4 |
| | | C288S | | | |
| t775505 | 17 | S92G V470A H133F | 19.6 | 0.61 | 41.7 |
| | | A433S C288S | | | |
| t775402 | 17 | S92G V470A M111V T40I | 19.5 | 7.10 | 40.9 |
| t775813 | 17 | S92G H133F A433S C288S | 19.3 | 4.34 | 103.8 |
| | | T40I | | | |
| t774299 | 17 | N258I I502V S499P | 19.2 | 2.17 | 65.3 |
| t775026 | 17 | S92G L432I C288S T40I | 19.2 | 1.58 | 140.6 |
| t774169 | 17 | S92G H133M I167K L432I | 18.9 | 3.85 | 62.8 |
| | | V470A N258R | | | |
| t775692 | 17 | S92G L432I V470A A263T | 18.1 | 6.10 | 92.0 |
| | | T40I | | | |
| t775566 | 17 | V470A A433S N258R T40I | 17.4 | 5.11 | 84.3 |
| t774414 | 17 | S92G H133M I167K | 16.7 | 1.81 | 43.9 |
| | | V470A A433S N258R | | | |
| t775558 | 17 | V470A H133F | 16.1 | 0.18 | 70.9 |
| t775853 | 17 | S92G V470A H133F | 16.0 | 1.45 | 69.6 |
| | | A433S N258I | | | |
| t775429 | 17 | S92G L432I V470A T40I | 15.8 | 1.35 | 14.5 |
| t775120 | 17 | V470A A433S A263T | 15.7 | 0.77 | 96.8 |
| | | M111V | | | |
| t775184 | 17 | S92G V470A A433S | 15.6 | 1.65 | 13.2 |
| | | N275S | | | |
| t774419 | 17 | S92G V470A A433S | 15.5 | 3.43 | 63.5 |
| | | N258R | | | |
| t775788 | 17 | S92G H133M I167K | 15.4 | 0.91 | 62.9 |
| | | V470A A263T | | | |
| t775656 | 17 | S92G L432I N258R | 15.0 | 0.42 | 59.1 |
| | | M111V T40I | | | |
| t775642 | 17 | S92G H133F N258R T40I | 14.9 | 2.86 | 57.9 |
| t774921 | 17 | H133M I167K V470A | 14.8 | 1.08 | 86.3 |
| | | N258I T40I | | | |
| t774339 | 17 | S92G V470A N258R | 14.1 | 2.94 | 49.3 |
| t774249 | 17 | S92G H133M I167K L432I | 12.7 | 0.29 | 34.7 |
| | | V470A T40I | | | |
| t775562 | 17 | H133M I167K V470A | 12.4 | 1.34 | 31.7 |
| | | N258R T40I | | | |
| t775114 | 17 | S92G H133M I167K | 11.9 | 0.55 | 49.1 |
| | | V470A A433S T40I | | | |
| t775058 | 17 | S92G H133M I167K | 11.1 | 2.47 | 39.5 |
| | | A433S N258I T40I | | | |
| t774639 | 17 | H133M I167K V470A | 11.1 | 0.40 | 17.8 |
| | | A433S | | | |
| t774883 | 17 | S92G H133M I167K | 11.0 | 1.15 | 37.9 |
| | | V470A A433S | | | |
| t775815 | 17 | H133M I167K V470A | 11.0 | 1.29 | 16.0 |
| | | A433S T40I | | | |
| t775602 | 17 | S92G L432I H133F | 10.5 | 0.76 | 11.1 |

Table 6 shows the secondary screen whole cell assay data of strains exhibiting >10% improved activity compared to the AvPAL positive control expression strain t773871 (AvPAL on an p1Aa vector).

TABLE 6
Data from secondary screen whole cell assay
(compared to positive control t773871)

| | | | Ave | Std | % Improved |
|---|---|---|---|---|---|
| | Template | Mutation | tCA | dev | to in-plate |
| Strain | SEQ ID | (relative to | (mM)/ | of CA/ | t773871 (WT |
| ID | NO: | Template) | OD600 | OD600 | AvPAL) |
| t775140 | 3 | G218A | 2.61 | 0.222 | 119.6 |
| t773872 | 3 | L4P G218S | 2.29 | 0.129 | 101.9 |
| t775301 | 3 | L219I L104M | 2.20 | 0.182 | 104.0 |
| t773870 | 3 | M222L | 1.87 | 0.206 | 41.1 |
| t774781 | 3 | L108T | 1.77 | 0.039 | 55.2 |
| t774777 | 3 | T102S | 1.74 | 0.048 | 52.9 |
| t775365 | 3 | N453S | 1.73 | 0.190 | 59.9 |
| t775132 | 3 | T102K | 1.68 | 0.185 | 26.0 |
| t774907 | 3 | T345S | 1.67 | 0.096 | 23.8 |
| t775063 | 3 | L108A | 1.62 | 0.098 | 60.8 |
| t774869 | 3 | D253A | 1.59 | 0.092 | 17.3 |
| t775036 | 3 | L219I L104M | 1.55 | 0.167 | 30.5 |
| | | M222T | | | |
| t774653 | 3 | L108Q | 1.55 | 0.122 | 30.3 |
| t774954 | 3 | I77V | 1.52 | 0.184 | 12.5 |
| t775337 | 3 | M222V | 1.50 | 0.220 | 54.4 |
| t774984 | 3 | A91V | 1.49 | 0.146 | 25.8 |
| t775377 | 3 | T102E | 1.48 | 0.073 | 51.9 |
| t774933 | 3 | A88S | 1.48 | 0.198 | 24.4 |
| t775866 | 3 | N453A | 1.47 | 0.132 | 45.8 |
| t774680 | 3 | N415H | 1.47 | 0.089 | 23.5 |
| t774833 | 3 | S175T | 1.45 | 0.131 | 27.6 |
| t775613 | 3 | L108H | 1.44 | 0.158 | 32.5 |
| t775438 | 3 | M222N | 1.44 | 0.147 | 33.5 |
| t775838 | 3 | A88S I423L | 1.44 | 0.129 | 32.0 |
| t775019 | 3 | A88S L104A | 1.43 | 0.166 | 42.6 |
| t775297 | 3 | V172A | 1.43 | 0.133 | 32.8 |
| t774795 | 3 | Q422H | 1.42 | 0.072 | 24.7 |
| t775502 | 3 | L407S | 1.39 | 0.108 | 43.1 |
| t775317 | 3 | A88S I165V | 1.37 | 0.075 | 41.2 |
| t775090 | 3 | L407A | 1.36 | 0.098 | 14.2 |
| t775507 | 3 | L407C | 1.35 | 0.127 | 18.3 |
| t775118 | 3 | M87L | 1.32 | 0.141 | 11.5 |
| t775286 | 3 | C424T | 1.31 | 0.096 | 15.5 |
| t775345 | 3 | L364H | 1.30 | 0.087 | 33.7 |
| t774577 | 3 | Y158H | 1.30 | 0.071 | 13.9 |
| t775516 | 3 | F450G | 1.29 | 0.188 | 19.1 |
| t775544 | 3 | S98A | 1.26 | 0.059 | 16.9 |
| t775401 | 3 | F450A | 1.25 | 0.099 | 29.0 |
| t775393 | 3 | T110S | 1.23 | 0.152 | 14.3 |
| t775353 | 3 | L219I L104A | 1.23 | 0.103 | 13.5 |
| | | L108T | | | |
| t775572 | 3 | L219I | 1.22 | 0.096 | 20.9 |
| t775357 | 3 | V90T | 1.22 | 0.144 | 12.5 |
| t775466 | 3 | V105F | 1.20 | 0.139 | 23.7 |
| t775595 | 3 | K413S | 1.19 | 0.149 | 10.4 |
| t775289 | 3 | L108V | 1.18 | 0.060 | 21.3 |
| t775807 | 3 | L407T | 1.18 | 0.034 | 17.2 |
| t774444 | 3 | L406V | 1.13 | 0.100 | 12.5 |
| t775812 | 3 | S175G | 1.12 | 0.117 | 15.5 |
| t774698 | 3 | F450H | 1.12 | 0.101 | 11.6 |
| t775573 | 3 | Y529L | 1.11 | 0.080 | 10.5 |

Table 7 shows the secondary screen cell lysate assay data of strains exhibiting >10% improved activity compared to the AvPAL positive control expression strain t773871 (AvPAL on an p15a vector).

TABLE 7
Data from secondary screen cell lysate assay
(compared to positive control t773871)

| | | | Ave OD | Std dev of OD | % Improved |
|---|---|---|---|---|---|
| | Template | Mutation | normalized | normalized | to in-plate |
| | SEQ ID | (relative to | rate (mA290/ | rate (mA290/ | t773871 (WT |
| Strain ID | NO: | Template | min/OD600) | min/OD600) | AvPAL) |
| t775301 | 3 | L219I L104M | 299.8 | 33.5 | 150.8 |
| t773870 | 3 | M222L | 265.0 | 34.1 | 96.5 |
| t774653 | 3 | L108Q | 261.6 | 23.6 | 155.0 |
| t775377 | 3 | T102E | 224.2 | 3.3 | 62.7 |
| t774907 | 3 | T345S | 219.4 | 23.4 | 79.2 |
| t774939 | 3 | T102R | 216.7 | 8.9 | 76.9 |
| t774954 | 3 | I77V | 213.0 | 9.9 | 73.9 |
| t775132 | 3 | T102K | 205.6 | 13.9 | 25.5 |
| t775502 | 3 | L407S | 200.7 | 13.8 | 45.6 |
| t775401 | 3 | F450A | 194.2 | 4.4 | 41.0 |
| t774718 | 3 | W106Q | 192.9 | 9.6 | 88.0 |
| t775365 | 3 | N453S | 192.0 | 6.1 | 60.6 |
| t774647 | 3 | F84Y | 191.8 | 7.5 | 87.0 |
| t775345 | 3 | L364H | 188.9 | 12.2 | 37.1 |
| t774834 | 3 | Y158V | 185.7 | 13.7 | 51.7 |
| t775516 | 3 | F450G | 185.6 | 6.0 | 55.3 |
| t775507 | 3 | L407C | 184.9 | 10.1 | 102.4 |
| t775297 | 3 | V172A | 183.9 | 39.5 | 53.9 |
| t774869 | 3 | D253A | 181.7 | 7.4 | 48.4 |
| t773872 | 3 | L4P G218S | 178.4 | 56.4 | 40.3 |
| t775357 | 3 | V90T | 176.6 | 8.8 | 47.7 |
| t774680 | 3 | N415H | 173.6 | 43.5 | 69.2 |
| t775595 | 3 | K413S | 168.5 | 6.7 | 41.0 |
| t774957 | 3 | K413G | 162.1 | 12.5 | 32.4 |
| t775261 | 3 | A394M | 160.6 | 11.7 | 34.4 |
| t775812 | 3 | S175G | 160.1 | 11.8 | 16.2 |
| t775199 | 3 | Q96E | 159.5 | 13.2 | 33.5 |
| t775289 | 3 | L108V | 157.6 | 4.7 | 14.4 |
| t774886 | 3 | M222I | 157.3 | 13.5 | 28.5 |
| t775393 | 3 | T110S | 151.9 | 7.4 | 27.1 |
| t775063 | 3 | L108A | 144.8 | 10.3 | 45.9 |
| t775544 | 3 | S98A | 143.5 | 7.3 | 20.1 |
| t775244 | 3 | L214Q | 136.2 | 27.2 | 40.1 |
| t775438 | 3 | M222N | 136.0 | 28.5 | 13.8 |
| t774515 | 3 | T243L | 132.9 | 35.0 | 33.9 |
| t775248 | 3 | K522P | 129.6 | 46.8 | 33.4 |
| t775140 | 3 | G218A | 128.6 | 7.4 | 32.4 |
| t774682 | 3 | L104A | 127.7 | 29.4 | 24.5 |
| t775118 | 3 | M87L | 126.4 | 49.7 | 30.1 |
| t774777 | 3 | T102S | 124.6 | 4.3 | 36.5 |
| t774833 | 3 | S175T | 107.1 | 2.6 | 17.3 |
| t774577 | 3 | Y158H | 104.8 | 4.4 | 14.7 |
| t775286 | 3 | C424T | 100.8 | 4.6 | 10.3 |

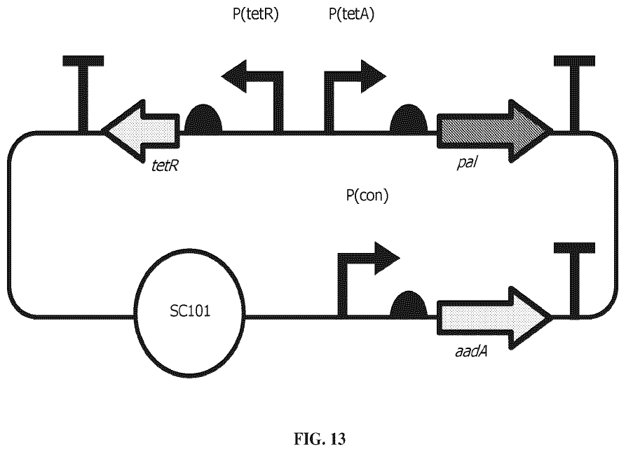

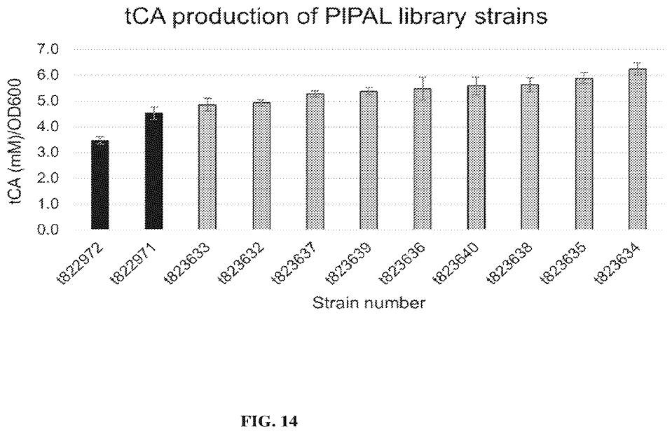

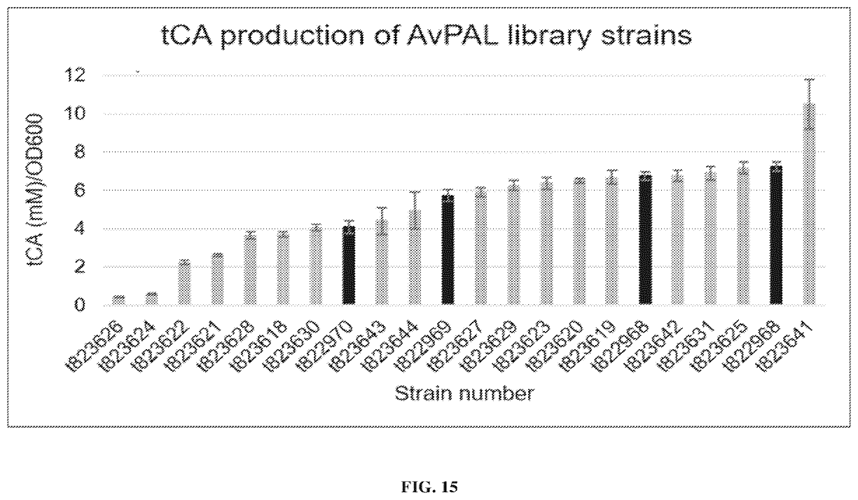

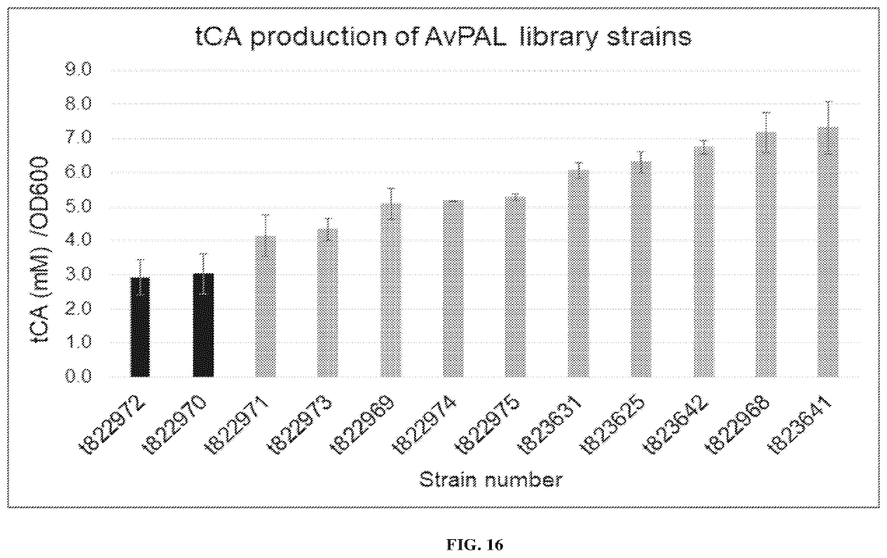

Four of the top AvPAL mutant hits, 9 P1PAL mutant hits and 14 AvPAL mutation recombinations (mutants with more than one substitution mutation) were selected for a tertiary screen. These were screened on a SC101-P(tet) vector (FIG. 13) in SYN107 as described in Example 1. The strains were induced at 33° C. and the whole cell assay was performed at 37° C. for 3 hours. 50 mg/L spectinomycin was used in place of kanamycin for growing the strain and selecting for transformants. P1PAL hits had comparable activity to the previously reported P1PAL mutants and improved activity over the WT enzyme (FIG. 14). The L219I L104M mutant-bearing strain from the secondary screen exhibited better activity when normalized to cell density (FIG. 15). All the mutants tested except for six of the AvPAL mutant recombinations showed >10% improvement over the WT P1PAL expression control (Table 8). Since the OD600 of strain t823641 (AvPAL L219I L104M) was ˜% of the other strains in the tertiary screen, a follow-up experiment was carried out on a subset of the most active AvPAL and P1PAL strains to ensure that all the strains were assayed within an OD₆₀₀ range from 0.5 to 0.8. Under these whole cell assay conditions, AvPAL L219I L104M was still the best performer, with activity similar to AvPAL L4P G218S. In general, the AvPAL mutants outperformed the P1PAL mutants, as the top five strains were AvPAL mutants and they exhibited ˜2× the activity of the WT PALs (FIG. 16).

Table 8 shows select tertiary screen hit strains with activity >10% higher than the P1PAL positive control expression strain t822972 (P1PAL on an SC101-specR vector).

TABLE 8
Data from tertiary screen (compared to positive control t822972)

| | | | | | % | |
|---|---|---|---|---|---|---|
| | | | | | improved to | % improved |
| | Temple | Mutation | Ave | | 1822972 | to t822970 |
| Strain | SEQ ID | (relative to | tCA(mM)/ | Std dev of | (PIPAL | (AvPAL |
| ID | NO: | Template) | OD600 | tCA/OD600 | WT) | WT) |
| t823641 | 3 | L219I L104M | 10.5 | 1.3 | 203.4 | 156.7 |
| t822968 | 3 | L4P G218S | 7.2 | 0.2 | 109.3 | 77.1 |
| t823625 | 3 | T102K G218A | 7.2 | 0.3 | 106.9 | 75.1 |
| t823631 | 3 | L104M L219I | 6.9 | 0.4 | 99.5 | 68.8 |
| | | M222L | | | | |
| t823642 | 3 | G218A | 6.8 | 0.3 | 95.7 | 65.6 |
| t822968 | 3 | L4P G218S | 6.7 | 0.2 | 94.8 | 64.9 |
| t823619 | 3 | T102S G218A | 6.7 | 0.4 | 93.3 | 63.6 |
| t823620 | 3 | T102K M222L | 6.5 | 0.1 | 87.9 | 59.1 |
| t823623 | 3 | L4P L104M | 6.4 | 0.3 | 83.8 | 55.6 |
| | | G218S | | | | |
| t823629 | 3 | G218A | 6.3 | 0.3 | 80.7 | 53.0 |
| | | L104M L219I | | | | |
| t823634 | 17 | C288S T40I | 6.2 | 0.2 | 79.8 | 52.2 |
| | | I502V S499P | | | | |
| t823627 | 3 | L104M L219I | 5.9 | 0.3 | 70.6 | 44.4 |
| | | N453S | | | | |
| t823635 | 17 | S92G L432I | 5.9 | 0.2 | 69.6 | 43.6 |
| | | V470A H133F | | | | |
| | | N258I | | | | |
| t822969 | 3 | M222L | 5.7 | 0.3 | 65.6 | 40.2 |
| t823638 | 17 | S92G L432I | 5.6 | 0.3 | 62.3 | 37.4 |
| | | V470A N258I | | | | |
| | | T40I | | | | |
| t823640 | 17 | S92G L432I | 5.6 | 0.3 | 60.9 | 36.2 |
| | | V470A T40I | | | | |
| t823636 | 17 | N2581 T40I | 5.5 | 0.4 | 58.0 | 33.7 |
| | | I502V S499P | | | | |
| t823639 | 17 | S92G V470A | 5.4 | 0.1 | 55.3 | 31.4 |
| | | M111V T40I | | | | |
| t823637 | 17 | S92G V470A | 5.3 | 0.1 | 52.1 | 28.7 |
| | | A263T T40I | | | | |
| t823644 | 3 | L108A | 4.9 | 1.0 | 42.9 | 20.9 |
| t823632 | 17 | S92G L432I | 4.9 | 0.1 | 42.1 | 20.3 |
| | | V470A N258R | | | | |
| | | T40I | | | | |
| t823633 | 17 | S92G L432I | 4.8 | 0.2 | 40.0 | 18.5 |
| | | M111V T40I | | | | |
| t822971 | 17 | N258R | 4.5 | 0.2 | 30.5 | 10.5 |
| t823643 | 3 | N453S | 4.4 | 0.7 | 27.2 | 7.6 |
| t822970 | 3 | WT | 4.1 | 0.3 | 18.2 | 0.0 |
| t823630 | 3 | G218S L4P | 4.1 | 0.2 | 17.1 | −0.9 |
| | | M222L | | | | |

### EQUIVALENTS

Those skilled in the art will recognize or be able to ascertain using no more than routine experimentation, many equivalents to the specific embodiments of the invention described in the present application. Such equivalents are intended to be encompassed by the following claims. All references, including patent documents, are incorporated by reference in their entirety.

1. A host cell that comprises a heterologous polynucleotide encoding a phenylalanine ammonia lyase (PAL) enzyme, wherein the PAL comprises a sequence that is at least 90% identical to any one of SEQ ID NOs: 1, 5, 7, 9, 11, 13 or 15, and wherein the host cell is not a plant cell.

2. The host cell of claim 1, wherein the PAL comprises the sequence of any one of SEQ ID NOs: 1, 5, 7, 9, 11, 13 or 15.

3. The host cell of claim 1 or 2, wherein the heterologous polynucleotide is at least 90% identical to any one of SEQ ID NOs: 2, 6, 8, 10, 12, 14 or 16.

4. The host cell of claim 3, wherein the heterologous polynucleotide comprises the sequence of any one of SEQ ID NOs: 2, 6, 8, 10, 12, 14 or 16.

5. The host cell of any one of claims 1-4, wherein the host cell is a bacterial cell, an archaebacterial cell, a fungal cell, a yeast cell, an animal cell, a mammalian cell, or a human cell.

6. The host cell of claim 5, wherein the host cell is a bacterial cell.

7. The host cell of claim 6, wherein the bacterial cell is an Escherichia coli (E. coli) cell.

8. The host cell of claim 6, wherein the bacterial cell is a Bacillus cell.

9. The host cell of any one of claims 1-5, wherein the host cell is a filamentous fungi cell or a yeast cell.

10. The host cell of claim 7, wherein the E. coli cell is an E. coli Nissle 1917 cell.

11. The host cell of any one of claims 1-10, wherein the PAL comprises one or more amino acid substitutions, additions, and/or deletions relative to the sequence of any one of SEQ ID NOs: 1, 5, 7, 9, 11, 13 or 15.

12. A host cell that comprises a heterologous polynucleotide encoding a phenylalanine ammonia lyase (PAL) enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529.

13. The host cell of claim 12, wherein the PAL comprises one or more of the following amino acid substitutions relative to SEQ ID NO: 3: I77V, F84Y, M87L, A88S, V90T, A91V, Q96E, S98A, V105F, W106Q, T110S, Y158H, Y158V, 1165V, V172A, S175G, S175T, L214Q, L219I, T243L, D253A, T345S, L364H, A394M, L406V, L407A, L407C, L407S, L407T, K413G, K413S, N415H, Q422H, I423L, C424T, F450A, F450G, F450H, N453A, N453S, K522P and Y529L.

14. The host cell of claim 12 or 13, wherein the PAL further comprises an amino acid substitution at one or more of the following amino acid residues relative to SEQ ID NO: 3: LA, T102, L104, L108, M222, G218 and N453.

15. The host cell of claim 14, wherein the PAL comprises one or more of the following amino acid substitutions relative to SEQ ID NO: 3: L4P, T102E, T102K, T102R, T102S, L104A, L104M, L108A, L108H, L108Q, L108T, L108V, M222I, M222L, M222N, M222T, M222V, G218A, G218S, N453A and N453S.

16. The host cell of any one of claims 12-15, wherein the PAL comprises an amino acid substitution at the following amino acid residues relative to SEQ ID NO: 3:
(i) L104 and L219;
(ii) L4 and G218;
(iii) T102 and G218;
(iv) L104, L219 and M222;
(v) T102 and G218;
(vi) T102 and M222;
(vii) L4, L104 and G218;
(viii) G218, L104 and L219;
(ix) L104, L219 and N453; or
(x) G218, L4 and M222.

17. The host cell of claim 16, wherein the PAL comprises any of the following relative to SEQ ID NO: 3:
(i) L104M and L219I;
(ii) L4P and G218S;
(iii) T102K and G218A;
(iv) L104M, L219I and M222L;
(v) T102S and G218A;
(vi) T102K and M222L;
(vii) L4P, L104M and G218S;
(viii) G218A, L104M and L219I;
(ix) L104M, L219I and N453S; or
(x) G218S, L4P and M222L.

18. The host cell of claim 17, wherein the PAL comprises the following amino acid substitutions relative to SEQ ID NO: 3: L104M and L219I.

19. The host cell of claim 17, wherein the PAL comprises the following amino acid substitution relative to SEQ ID NO: 3: L4P and G218S.

20. A host cell that comprises a heterologous polynucleotide encoding a phenylalanine ammonia lyase (PAL) enzyme, wherein the PAL comprises an amino acid substitution at one or more of the following amino acid residues relative to SEQ ID NO: 17: T40, M111, N258, N275, C288, M402, S499 and I502.

21. The host cell of claim 20, wherein the PAL comprises one or more of the following amino acid substitutions relative to SEQ ID NO: 17: T40I, M111V, N258I, N258R, N275S, C288S, M402L, S499P and I502V.

22. The host cell of claim 20 or 21, wherein the PAL further comprises an amino acid substitution at one or more of the following amino acid residues relative to SEQ ID NO: 17: S92, H133, 1167, A263, L432, A433 and V470.

23. The host cell of claim 22, wherein the PAL further comprises one or more of the following amino acid substitutions relative to SEQ ID NO: 17: S92G, H133F, H133M, I167K, A263T, L432I, A433S and V470A.

24. The host cell of any one of claims 20-23, wherein the PAL comprises an amino acid substitution at the following amino acid residues relative to SEQ ID NO: 17:
(i) C288, T40, 1502 and S499;
(ii) S92, L432, V460, H133 and N258;
(iii) S92, L432, V470, N258 and T40;
(iv) S92, L432, V470 and T40;
(v) N258, T40, 1502 and S499;
(vi) S92, V470, M111 and T40;
(vii) S92, L432, V470, N258 and T40;
(viii) S92, V470, A263 and T40; or
(ix) S92, L432, M111 and T40.

25. The host cell of claim 24, wherein the PAL comprises any one of the following relative to SEQ ID NO: 17:
(i) C288S, T40I, I502V and S499P;
(ii) S92G, L432I, V460A, H133F and N258I;
(iii) S92G, L432I, V470A, N258I and T40I;
(iv) S92G, L432I, V470A and T40I;
(v) N258I, T40I, I502V and S499P;
(vi) S92G, V470A, M111V and T40I;
(vii) S92G, L432I, V470A, N258R and T40I;
(viii) S92G, V470A, A263T and T40I; or
(ix) S92G, L432I, M111V and T40I.

26. The host cell of any one of claims 20-25, wherein the PAL comprises the amino acid substitution N258R relative to SEQ ID NO: 17.

27. The host cell of claim 25, wherein the PAL comprises the following amino acid substitutions relative to SEQ ID NO: 17: T40I, C288S, S499P and I502V.

28. The host cell of any one of claims 12-27, wherein the host cell is a bacterial cell.

29. The host cell of claim 28, wherein the bacterial cell is an Escherichia coli (E. coli) cell.

30. The host cell of claim 28, wherein the bacterial cell is a Bacillus cell.

31. The host cell of any one of claims 12-27, wherein the host cell is a filamentous fungi cell or a yeast cell.

32. The host cell of claim 29, wherein the E. coli cell is an E. coli Nissle 1917 cell.

33. The host cell of any one of claims 1-32, wherein the PAL is able to convert phenylalanine to trans-cinnamic acid.

34. A method of treating or protecting against a metabolic disorder associated with excess phenylalanine, comprising administering to a subject in need thereof a therapeutically effective amount of a phenylalanine ammonia lyase (PAL) enzyme or a polynucleotide encoding a PAL enzyme, wherein the PAL comprises a sequence that is at least 90% identical to any one of SEQ ID NOs: 1, 5, 7, 9, 11, 13 or 15.

35. A method of treating or protecting against a metabolic disorder associated with excess phenylalanine, comprising administering to a subject in need thereof a cell comprising a heterologous polynucleotide encoding a therapeutically effective amount of a phenylalanine ammonia lyase (PAL) enzyme, wherein the PAL comprises a sequence that is at least 90% identical to any one of SEQ ID NOs: 1, 5, 7, 9, 11, 13 or 15.

36. The method of claim 35, wherein the cell is a human cell, an animal cell, a yeast cell, or a bacterial cell.

37. A method of converting phenylalanine to trans-cinnamic acid, comprising contacting phenylalanine with a phenylalanine ammonia lyase (PAL) enzyme, wherein the PAL comprises a sequence that is at least 90% identical to any one of SEQ ID NOs: 1, 5, 7, 9, 11, 13 or 15.

38. The method of claim 37, wherein the PAL is encoded by a polynucleotide comprising a sequence that is at least 90% identical to any one of SEQ ID NO: 2, 6, 8, 10, 12, 14 or 16.

39. The method of any one of claims 35-38, wherein the PAL comprises one or more amino acid substitutions relative to the sequence of any one of SEQ ID NOs: 1, 5, 7, 9, 11, 13 or 15.

40. A method of treating or protecting against a metabolic disorder associated with excess phenylalanine, comprising administering to a subject in need thereof a therapeutically effective amount of a phenylalanine ammonia lyase (PAL) enzyme or a polynucleotide encoding a PAL enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, LA06, L407, K413, N415, Q422, I423, C424, F450, K522 and Y529.

41. A method of treating or protecting against a metabolic disorder associated with excess phenylalanine, comprising administering to a subject in need thereof a cell comprising a heterologous polynucleotide encoding a therapeutically effective amount of a phenylalanine ammonia lyase (PAL) enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529.

42. The method of claim 41, wherein the cell is a human cell, an animal cell, a yeast cell, or a bacterial cell.

43. A method of converting phenylalanine to trans-cinnamic acid, comprising contacting phenylalanine with a phenylalanine ammonia lyase (PAL) enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, 1423, C424, F450, K522 and Y529.

44. The method of any one of claims 40-43, wherein the PAL comprises one or more of the following amino acid substitutions relative to SEQ ID NO: 3: I77V, F84Y, M87L, A88S, V90T, A91V, Q96E, S98A, V105F, W106Q, T110S, Y158H, Y158V, 1165V, V172A, S175G, S175T, L214Q, L219I, T243L, D253A, T345S, L364H, A394M, L406V, L407A, L407C, L407S, L407T, K413G, K413S, N415H, Q422H, I423L, C424T, F450A, F450G, F450H, N453A, N453S, K522P and Y529L.

45. The method of any one of claims 40-44, wherein the PAL further comprises an amino acid substitution at one or more of the following amino acid residues relative to SEQ ID NO: 3: L4, T102, L104, L108, M222, G218 and N453.

46. The method of claim 45, wherein the PAL comprises one or more of the following amino acid substitutions relative to SEQ ID NO: 3: L4P, T102E, T102K, T102R, T102S, L104A, L104M, L108A, L108H, L108Q, L108T, L108V, M222I, M222L, M222N, M222T, M222V, G218A, G218S, N453A and N453S.

47. The method of any one of claims 40-46, wherein the PAL comprises an amino acid substitution at the following amino acid residues relative to SEQ ID NO: 3:
(i) L104 and L219;
(ii) L4 and G218;
(iii) T102 and G218;
(iv) L104, L219 and M222;
(v) T102 and G218;
(vi) T102 and M222;
(vii) L4, L104 and G218;
(viii) G218, L104 and L219;
(ix) L104, L219 and N453; or
(x) G218, L4 and M222.

48. The method of claim 47, wherein the PAL comprises any of the following relative to SEQ ID NO: 3:
(i) L104M and L219I;
(ii) L4P and G218S;
(iii) T102K and G218A;
(iv) L104M, L219I and M222L;
(v) T102S and G218A;
(vi) T102K and M222L;
(vii) L4P, L104M and G218S;
(viii) G218A, L104M and L219I;
(ix) L104M, L219I and N453S; or
(x) G218S, L4P and M222L.

49. The method of claim 48, wherein the PAL comprises the following amino acid substitutions relative to SEQ ID NO: 3: L104M and L219I.

50. The method of claim 48, wherein the PAL comprises the following amino acid substitution relative to SEQ ID NO: 3: L4P and G218S.

51. A method of treating or protecting against a metabolic disorder associated with excess phenylalanine, comprising administering to a subject in need thereof a therapeutically effective amount of a phenylalanine ammonia lyase (PAL) enzyme or a polynucleotide encoding a PAL enzyme, wherein the PAL comprises an amino acid substitution at one or more of the following amino acid residues relative to SEQ ID NO: 17: T40, M111, N258, N275, C288, M402, S499 and I502.

52. The method of claim 51, wherein the PAL further comprises an amino acid substitution at one or more of the following amino acid residues relative to SEQ ID NO: 17: S92, H133, 1167, A263, L432, A433 and V470.

53. A method of treating or protecting against a metabolic disorder associated with excess phenylalanine, comprising administering to a subject in need thereof a cell comprising a heterologous polynucleotide encoding a therapeutically effective amount of a phenylalanine ammonia lyase (PAL) enzyme, wherein the PAL comprises an amino acid substitution at one or more of the following amino acid residues relative to SEQ ID NO: 17: T40, M111, N258, N275, C288, M402, S499 and I502.

54. The method of claim 53, wherein the PAL further comprises an amino acid substitution at one or more of the following amino acid residues relative to SEQ ID NO: 17: S92, H133, 1167, A263, L432, A433 and V470.

55. The method of claim 53 or 54, wherein the cell is a human cell, an animal cell, a yeast cell, or a bacterial cell.

56. A method of converting phenylalanine to trans-cinnamic acid, comprising contacting phenylalanine with a phenylalanine ammonia lyase (PAL) enzyme, wherein the PAL comprises an amino acid substitution at one or more of the following amino acid residues relative to SEQ ID NO: 17: T40, M111, N258, N275, C288, M402, S499 and I502.

57. The method of claim 56, wherein the PAL further comprises an amino acid substitution at one or more of the following amino acid residues relative to SEQ ID NO: 17: S92, H133, 1167, A263, L432, A433 and V470.

58. The method of any one of claims 51-57, wherein the PAL comprises one or more of the following amino acid substitutions relative to SEQ ID NO: 17: T40I, M111V, N258I, N258R, N275S, C288S, M402L, S499P and I502V.

59. The method of claim 58, wherein the PAL further comprises one or more of the following amino acid substitutions relative to SEQ ID NO: 17: S92G, H133F, H133M, I167K, A263T, L432I, A433S and V470A.

60. The method of any one of claims 51-59, wherein the PAL comprises an amino acid substitution at the following amino acid residues relative to SEQ ID NO: 17:
(i) C288, T40, 1502 and $499;
(ii) S92, L432, V460, H133 and N258;
(iii) S92, L432, V470, N258 and T40;
(iv) S92, L432, V470 and T40;
(v) N258, T40, 1502 and S499;
(vi) S92, V470, M111 and T40;
(vii) S92, L432, V470, N258 and T40;
(viii) S92, V470, A263 and T40; or
(ix) S92, L432, M111 and T40.

61. The method of claim 60, wherein the PAL comprises any one of the following relative to SEQ ID NO: 17:
(i) C288S, T40I, I502V and S499P;
(ii) S92G, L432I, V460A, H133F and N258I;
(iii) S92G, L432I, V470A, N258I and T40I;
(iv) S92G, L432I, V470A and T40I;
(v) N258I, T40I, I502V and S499P;
(vi) S92G, V470A, M111V and T40I;
(vii) S92G, L432I, V470A, N258R and T40I;
(viii) S92G, V470A, A263T and T40I; or
(ix) S92G, L432I, M111V and T40I.

62. The method of any one of claims 51-61, wherein the PAL comprises the amino acid substitution N258R relative to SEQ ID NO: 17.

63. The method of claim 61, wherein the PAL comprises the following amino acid substitutions relative to SEQ ID NO: 17: T40I, C288S, S499P and I502V.

64. The method of any one of claims 34-63, wherein the method is a method of protecting a subject against phenylketonuria or hyperphenylalaninemia.

65. The method of any one of claims 34-63, wherein the method is a method of treating a subject that has phenylketonuria or hyperphenylalaninemia.

66. A phenylalanine ammonia lyase (PAL) enzyme, wherein the PAL comprises an amino acid substitution at one or more of the following amino acid residues relative to SEQ ID NO: 3: I77, F84, M87, A88, V90, A91, Q96, S98, L104, V105, W106, T110, Y158, I165, V172, S175, L214, L219, T243, D253, T345, L364, A394, L406, L407, K413, N415, Q422, I423, C424, F450, K522 and Y529.

67. The PAL enzyme of claim 66, wherein the PAL comprises one or more of the following amino acid substitutions relative to SEQ ID NO: 3: I77V, F84Y, M87L, A88S, V90T, A91V, Q96E, S98A, V105F, W106Q, T110S, Y158H, Y158V, 1165V, V172A, S175G, S175T, L214Q, L219I, T243L, D253A, T345S, L364H, A394M, L406V, L407A, L407C, L407S, L407T, K413G, K413S, N415H, Q422H, I423L, C424T, F450A, F450G, F450H, N453A, N453S, K522P and Y529L.

68. The PAL enzyme of claim 66 or 67, wherein the PAL further comprises an amino acid substitution at one or more of the following amino acid residues relative to SEQ ID NO: 3: L4, T102, L104, L108, M222, G218 and N453.

69. The PAL enzyme of claim 68, wherein the PAL comprises one or more of the following amino acid substitutions relative to SEQ ID NO: 3: L4P, T102E, T102K, T102R, T102S, L104A, L104M, L108A, L108H, L108Q, L108T, L108V, M222I, M222L, M222N, M222T, M222V, G218A, G218S, N453A and N453S.

70. The PAL enzyme of any one of claims 66-69, wherein the PAL comprises an amino acid substitution at the following amino acid residues relative to SEQ ID NO: 3:
(i) L104 and L219;
(ii) L4 and G218;
(iii) T102 and G218;
(iv) L104, L219 and M222;
(v) T102 and G218;
(vi) T102 and M222;
(vii) L4, L104 and G218;
(viii) G218, L104 and L219;
(ix) L104, L219 and N453; or
(x) G218, L4 and M222.

71. The PAL enzyme of claim 70, wherein the PAL comprises any of the following relative to SEQ ID NO: 3:
(i) L104M and L219I;
(ii) L4P and G218S;
(iii) T102K and G218A;
(iv) L104M, L219I and M222L;
(v) T102S and G218A;
(vi) T102K and M222L;
(vii) L4P, L104M and G218S;
(viii) G218A, L104M and L219I;
(ix) L104M, L219I and N453S; or
(x) G218S, L4P and M222L.

72. The PAL enzyme of claim 71, wherein the PAL comprises the following amino acid substitutions relative to SEQ ID NO: 3: L104M and L219I.

73. The PAL enzyme of claim 71, wherein the PAL comprises the following amino acid substitution relative to SEQ ID NO: 3: L4P and G218S.

74. A phenylalanine ammonia lyase (PAL) enzyme, wherein the PAL comprises an amino acid substitution at one or more of the following amino acid residues relative to SEQ ID NO: 17: T40, M111, N258, N275, C288, M402, S499 and I502.

75. The PAL enzyme of claim 74, wherein the PAL comprises one or more of the following amino acid substitutions relative to SEQ ID NO: 17: T40I, M111V, N258I, N258R, N275S, C288S, M402L, S499P and I502V.

76. The PAL enzyme of claim 74 or 75, wherein the PAL enzyme further comprises an amino acid substitution at one or more of the following amino acid residues relative to SEQ ID NO: 17: S92, H133, 1167, A263, L432, A433 and V470.

77. The PAL enzyme of claim 76, wherein the PAL enzyme further comprises one or more of the following amino acid substitutions relative to SEQ ID NO: 17: S92G, H133F, H133M, I167K, A263T, L432I, A433S and V470A.

78. The PAL enzyme of any one of claims 74-77, wherein the PAL comprises an amino acid substitution at the following amino acid residues relative to SEQ ID NO: 17:
(i) C288, T40, 1502 and S499;
(ii) S92, L432, V460, H133 and N258;
(iii) S92, L432, V470, N258 and T40;
(iv) S92, L432, V470 and T40;
(v) N258, T40, 1502 and $499;
(vi) S92, V470, M111 and T40;
(vii) S92, L432, V470, N258 and T40;
(viii) S92, V470, A263 and T40; or
(ix) S92, L432, M111 and T40.

79. The PAL enzyme of claim 78, wherein the PAL comprises any one of the following relative to SEQ ID NO: 17:
(i) C288S, T40I, I502V and S499P;
(ii) S92G, L432I, V460A, H133F and N258I;
(iii) S92G, L432I, V470A, N258I and T40I;
(iv) S92G, L432I, V470A and T40I;
(v) N258I, T40L, I502V and S499P;
(vi) S92G, V470A, M111V and T40I;
(vii) S92G, L432I, V470A, N258R and T40I;
(viii) S92G, V470A, A263T and T40I; or
(ix) S92G, L432I, M111V and T40I.

80. The PAL enzyme of any one of claims 74-79, wherein the PAL comprises the amino acid substitution N258R relative to SEQ ID NO: 17.

81. The PAL enzyme of claim 79, wherein the PAL comprises the following amino acid substitutions relative to SEQ ID NO: 17: T40I, C288S, S499P and I502V.
